(19) United States
(12) Patent Application Publication
(10) Pub. No.: US 2026/0341476 A1
(43) Pub. Date: Oct. 29, 2026

(54) MULTIMODAL MACHINE LEARNING TECHNIQUES FOR GENERATING AND IMPUTING BIOLOGICAL DATA

(71) Applicant: BostonGene Corporation, Waltham, MA (US)

(72) Inventors: Viktor Svekolkin, Yerevan (AM); Aleksandr Sarachakov, Yerevan (AM); Gor Chobanyan, Yerevan (AM); Alexander Bagaev, Waltham, MA (US); Michael Feinberg, Lincoln, MA (US)

(21) Appl. No.: 19/658,313

(22) Filed: Apr. 24, 2026

Related U.S. Application Data
(60) Provisional application No. 63/795,145, filed on Apr. 25, 2025.
(60) Provisional application No. 64/004,545, filed on Mar. 13, 2026.

(51) Int. Cl. G16H 30/40; G06F 18/2132; G06N 3/0455; G06N 3/0464; G16B 25/10; G16B 45/00; G16H 20/10; G16H 50/70

(52) U.S. Cl. CPC G16H 30/40; G06F 18/21324; G06N 3/0455; G06N 3/0464; G16B 25/10; G16B 45/00; G16H 20/10; G16H 50/70

(57) ABSTRACT

Aspects of the disclosure relate to techniques for generating data of a target modality for a subject. In some embodiments, the techniques include: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

### CROSS-REFERENCE TO RELATED APPLICATIONS

This application claims the benefit of and priority under 35 U.S.C. § 119 (e) of the filing date of U.S. Provisional Application No. 63/795,145, filed Apr. 25, 2025, and entitled “MULTI-OMICS MACHINE LEARNING TECHNIQUES FOR INTEGRATING AND GENERATING DATA,” and U.S. Provisional Application No. 64/004,545, filed Mar. 13, 2026, and entitled “VISUALIZING THE GENOTYPE-PHENOTYPE LINK: PREDICTING DRUG-INDUCED TISSUE DYNAMICS WITH RNA-BASED DIFFUSION MODELS,” each of which is incorporated by reference herein in its entirety.

### BACKGROUND

The biological state of a subject affects how cancers and other diseases develop, progress, and respond to therapy. The biological state reflects the combined activity of multiple interacting biological systems. Advances in technology have made it possible to measure many aspects of these systems, producing a diverse range of data modalities that together provide a more complete picture of the biological state of a subject than any single measurement alone. Examples of different data modalities include genomics, transcriptomics, imaging, cytometry, spatial omics, microbiome, proteomics, metabolomics, epigenomics, and clinical data, among others.

### SUMMARY

Some aspects provide for a method for generating data of a target modality for a subject, the method comprising: using at least one processor to perform: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

Some aspects provide for a system, comprising: at least one processor; and at least one non-transitory computer-readable storage medium storing processor-executable instructions that, when executed by the at least one processor, cause the at least one processor to perform a method for generating data of a target modality for a subject, the method comprising: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

Some aspects provide for at least one non-transitory computer-readable storage medium storing processor-executable instructions that, when executed by at least one processor, cause the at least one processor to perform a method for generating data of a target modality for a subject, the method comprising: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

Some aspects provide for a method for imputing missing modality data for a subject, the method comprising: using at least one processor to perform: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some aspects provide for a system, comprising: at least one processor; and at least one non-transitory computer-readable storage medium storing processor-executable instructions that, when executed by the at least one processor, cause the at least one processor to perform a method for imputing missing modality data for a subject, the method comprising: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some aspects provide for at least one non-transitory computer-readable storage medium storing processor-executable instructions that, when executed by at least one processor, cause the at least one processor to perform a method for imputing missing modality data for a subject, the method comprising: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some aspects provide for a method for generating, from RNA expression data, a tissue image for a subject, the method comprising: using at least one processor to perform: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

Some aspects provide for a system, comprising: at least one processor; and at least one non-transitory computer-readable storage medium storing processor-executable instructions that, when executed by the at least one processor, cause the at least one processor to perform a method for generating, from RNA expression data, a tissue image for a subject, the method comprising: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

Some aspects provide for at least one non-transitory computer-readable storage medium storing processor-executable instructions that, when executed by at least one processor, cause the at least one processor to perform a method for generating, from RNA expression data, a tissue image for a subject, the method comprising: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

Some aspects provide for a method for generating, from a tissue image, a representation of RNA expression data for a subject and an output based on the representation of RNA expression data, the method comprising: using at least one processor to perform: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some aspects provide for a system, comprising: at least one processor; and at least one non-transitory computer-readable storage medium storing processor-executable instructions that, when executed by the at least one processor, cause the at least one processor to perform a method for generating, from a tissue image, a representation of RNA expression data for a subject and an output based on the representation of RNA expression data, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some aspects provide for at least one non-transitory computer-readable storage medium storing processor-executable instructions that, when executed by at least one processor, cause the at least one processor to perform a method for generating, from a tissue image, a representation of RNA expression data for a subject and an output based on the representation of RNA expression data, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some aspects provide for a method for generating, from a tissue image, a plurality of output values for a subject, the method comprising: using at least one processor to perform: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some aspects provide for a system, comprising: at least one processor; and at least one non-transitory computer-readable storage medium storing processor-executable instructions that, when executed by the at least one processor, cause the at least one processor to perform a method for generating, from a tissue image, a plurality of output values for a subject, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some aspects provide for at least one non-transitory computer-readable storage medium storing processor-executable instructions that, when executed by at least one processor, cause the at least one processor to perform a method for generating, from a tissue image, a plurality of output values for a subject, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some aspects provide for a method for training a multimodal generative model to generate data of a target modality for a subject, the method comprising: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

Some aspects provide for a system, comprising: at least one processor; and at least one non-transitory computer-readable storage medium storing processor-executable instructions that, when executed by the at least one processor, cause the at least one processor to perform a method for training a multimodal generative model to generate data of a target modality for a subject, the method comprising: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise. Some aspects provide for at least one non-transitory computer-readable storage medium storing processor-executable instructions that, when executed by at least one processor, cause the at least one processor to perform a method for training a multimodal generative model to generate data of a target modality for a subject, the method comprising: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

### BRIEF DESCRIPTION OF THE FIGURES

FIG. 1A is a diagram of an illustrative technique for generating data of a target modality for a subject, according to some embodiments of the technology described herein.

FIG. 1B is a diagram of an illustrative technique for generating a tissue image from RNA expression data for a subject, according to some embodiments of the technology described herein.

FIG. 1C is a diagram of an illustrative technique for generating, from a tissue image, a representation of RNA expression data and an output based on the representation of the RNA expression data for a subject, according to some embodiments of the technology described herein.

FIG. 1D is a diagram of an illustrative technique for generating, from a tissue image, output values for a subject, according to some embodiments of the technology described herein.

FIG. 2 is a block diagram of an example system for generating data of a target modality for a subject, according to some embodiments of the technology described herein.

FIG. 3A is a flowchart of an illustrative process for generating data of a target modality for a subject, according to some embodiments of the technology described herein.

FIG. 3B is a flowchart of an illustrative process for generating a tissue image from RNA expression data for a subject, according to some embodiments of the technology described herein.

FIG. 3C is a flowchart of an illustrative process for generating, from a tissue image, a representation of RNA expression data and an output based on the representation of the RNA expression data for a subject, according to some embodiments of the technology described herein.

FIG. 3D is a flowchart of an illustrative process for generating, from a tissue image, output values for a subject, according to some embodiments of the technology described herein.

FIG. 3E is a flowchart of an illustrative process for training a multimodal generative model to generate data of a target modality for a subject, according to some embodiments of the technology described herein.

FIG. 4A-1, FIG. 4A-2, FIG. 4B-1, FIG. 4B-2, FIG. 4B-3 and FIG. 4C are examples of integrating multiple data modalities to generate data of a target modality, according to some embodiments of the technology described herein.

FIG. 4D is a diagram of example encoders used for encoding data of multiple input modalities, according to some embodiments of the technology described herein.

FIG. 4E is a diagram of example adapters and a multimodal generative models used to generate data of a target modality, according to some embodiments of the technology described herein.

FIG. 5A is a diagram showing an example of using a multimodal generative model to simulate effects of biological alterations on a subject's multimodal data profile, according to some embodiments of the technology described herein.

FIG. 5B is a diagram showing an example of using a multimodal generative model to model a subject's trajectory through a high-dimensional state landscape, according to some embodiments of the technology described herein.

FIG. 5C and FIG. 5D show an example of using a multimodal generative model to simulate the effects of a therapeutic agent on the subject, according to some embodiments of the technology described herein.

FIG. 5E shows an example of using a multimodal generative model to impute missing pathology data from blood analysis, according to some embodiments of the technology described herein.

FIG. 5F shows an example of using a multimodal generative model to impute missing genomic data from hematoxylin and eosin (H&E) images, according to some embodiments of the technology described herein.

FIG. 6A is a diagram of an example technique for generating a tissue image from RNA expression data, according to some embodiments of the technology described herein.

FIG. 6B shows an example architecture of a denoising diffusion model (e.g., denoising diffusion model 607 shown in FIG. 6A) used to generate a representation of a tissue image, according to some embodiments of the technology described herein.

FIG. 6C shows an example architecture of a downsampling block (e.g., downsampling blocks 614, 615, and 616 shown in FIG. 6B) of the denoising diffusion model shown in FIG. 6B, according to some embodiments of the technology described herein.

FIG. 6D shows an example architecture of a bottleneck block (e.g., bottleneck block 618 shown in FIG. 6B) of the denoising diffusion model shown in FIG. 6B, according to some embodiments of the technology described herein.

FIG. 6E shows an example architecture of an upsampling block (e.g., upsampling blocks 620, 621, and 622 shown in FIG. 6B) of the denoising diffusion model shown in FIG. 6B, according to some embodiments of the technology described herein.

FIG. 6F shows an example architecture of a cross-attention layer of the downsampling block shown in FIG. 6C, the bottleneck block shown in FIG. 6D, and the upsampling block shown in FIG. 6E, according to some embodiments of the technology described herein.

FIG. 6G shows an example architecture of an encoder (e.g., trained encoder 602 shown in FIG. 6A) used to encode RNA expression data, according to some embodiments of the technology described herein.

FIG. 6H shows an example architecture of a residual block (e.g., residual blocks 649, 650, 651, 652, and 653 shown in FIG. 6G) of the encoder shown in FIG. 6G, according to some embodiments of the technology described herein.

FIG. 6I shows an example architecture of an encoder (e.g., trained encoder 602 shown in FIG. 6A) used to encode RNA expression data, according to some embodiments of the technology described herein.

FIG. 6J shows an example architecture of an adapter (e.g., trained adapter 604 shown in FIG. 6A), according to some embodiments of the technology described herein.

FIG. 7A, FIG. 7B, and FIG. 7C show examples of tissue images generated, according to some embodiments of the technology described herein, from RNA expression data.

FIG. 7D and FIG. 7E are plots showing the performance of an example denoising diffusion model in generating tissue images that match corresponding ground truth tissue images. Lower Fréchet inception distance (FID) values suggest that the distribution of generated images closely matches that of the corresponding ground truth images.

FIG. 7F, FIG. 7G, FIG. 7H, and FIG. 7I are examples showing tissue images generated from RNA expression data that has been perturbed, according to some embodiments of the technology described herein.

FIG. 7J is a diagram showing an examples of using a multimodal generative model to simulate alternative biological trajectories based on perturbations of RNA expression data, according to some embodiments of the technology described herein.

FIG. 7K shows an illustration of an end-to-end inference of tissue images from RNA expression data, according to some embodiments of the technology described herein.

FIG. 8A is a diagram of an example technique for generating, from a tissue image, a representation of RNA expression data and output obtained from the representation of the RNA expression data, according to some embodiments of the technology described herein.

FIG. 8B shows an example architecture of an encoder (e.g., trained encoder 805 shown in FIG. 8A) used to encode image portions, according to some embodiments of the technology described herein.

FIG. 8C shows an example architecture of an adapter (e.g., trained adapter 807 shown in FIG. 8A), according to some embodiments of the technology described herein.

FIG. 8D shows an example architecture of a denoising diffusion model (e.g., denoising diffusion model 810 shown in FIG. 8A) used to generate a representation of RNA expression data, according to some embodiments of the technology described herein.

FIG. 8E shows an example architecture of a down block (e.g., downsampling blocks 828 and 829 shown in FIG. 8D) of the denoising diffusion model shown in FIG. 8D, according to some embodiments of the technology described herein.

FIG. 8F shows an example architecture of a down block (e.g., down block 830 shown in FIG. 8D) of the denoising diffusion model shown in FIG. 8D, according to some embodiments of the technology described herein.

FIG. 8G shows an example architecture of a mid block (e.g., mid block 831 shown in FIG. 8D) of the denoising diffusion model shown in FIG. 8D, according to some embodiments of the technology described herein.

FIG. 8H shows an example architecture of an up block (e.g., up block 832 shown in FIG. 8D) of the denoising diffusion model shown in FIG. 8D, according to some embodiments of the technology described herein.

FIG. 8I shows an example architecture of an up block (e.g., up block 833 shown in FIG. 8D) of the denoising diffusion model shown in FIG. 8D, according to some embodiments of the technology described herein.

FIG. 8J shows an example architecture of an up block (e.g., up block 834 shown in FIG. 8D) of the denoising diffusion model shown in FIG. 8D, according to some embodiments of the technology described herein.

FIG. 8K shows an example architecture of a time embedding network used to generate a time embedding (e.g., time embedding 838 shown in FIG. 8D) used by the denoising diffusion model shown in FIG. 8D, according to some embodiments of the technology described herein.

FIG. 9 shows an example of a tissue image and a plot showing that transcriptomic signature scores derived from RNA expression data generated from the tissue image, according to some embodiments of the technology described herein, closely match corresponding ground truth signature scores.

FIG. 10A is a diagram of an example technique for generating, from a tissue image, signature score(s) for gene group(s) for a subject, according to some embodiments of the technology described herein.

FIG. 10B shows an example architecture of an adapter (e.g., trained adapter 1008 shown in FIG. 10A), according to some embodiments of the technology described herein.

FIG. 10C shows an example architecture of a feature extraction network (e.g., feature extraction network 1012 shown in FIG. 10B) of the adapter shown in FIG. 10B, according to some embodiments of the technology described herein.

FIG. 10D shows an example architecture of a gated attention network (e.g., gated attention network 1014 shown in FIG. 10B) of the adapter shown in FIG. 10B, according to some embodiments of the technology described herein.

FIG. 11A, FIG. 11B, and FIG. 11C show examples of heatmaps generated as a result of generating signature scores according to some embodiments of the technology describe herein.

FIG. 12 is a schematic diagram of an illustrative computing system with which aspects described herein may be implemented.

### DETAILED DESCRIPTION

A comprehensive characterization of a patient's biological state can draw upon data from multiple modalities, including, for example, RNA expression data (e.g., RNA-sequencing data), histopathological images (e.g., hematoxylin and eosin stained tissue slides), genomic profiles (e.g., somatic mutation and copy number data), immune cell profiling data (e.g., flow cytometry, T-cell receptor sequencing, B-cell receptor sequencing), cell-free nucleic acid measurements (e.g., cfRNA and cfDNA data), and clinical data (e.g., tabular clinical metadata and textual medical notes), among others. Different modalities capture complementary dimensions of disease biology across biological compartments, including tumor-intrinsic state, the tumor microenvironment, peripheral and tissue-resident immune state, circulating molecular signals, and systemic clinical phenotype. For example, histopathological images reflect tissue and cellular composition, while RNA expression data captures transcriptional state. As another example, genomic profiles reveal mutational burden and driver alterations, while immune cell profiling data characterizes the immune microenvironment. Together, these modalities support a wide range of clinically important analyses, from characterizing the tumor microenvironment and predicting treatment response to stratifying patients for clinical trials and monitoring disease progression.

In practice, however, comprehensive multimodal data is rarely available for any given patient. The cost, invasiveness, and logistical complexity of obtaining biological samples mean that different patients typically have different subsets of data modalities available. Tissue images may be available for a subject without corresponding RNA expression data, or RNA expression data may be available without corresponding tissue images. RNA expression data may be unavailable because: (i) RNA sequencing is a non-routine assay ordered selectively, (ii) formalin fixation degrades RNA such that sequencing of archival tissue is unreliable, and/or (iii) small biopsies may be entirely consumed by other analyses, leaving little or no material for sequencing. Tissue image data may be unavailable because: (i) tumors in inaccessible sites such as the brain may preclude tissue sampling altogether, and/or (ii) in longitudinal monitoring settings, repeated tissue biopsies may be infeasible due to invasiveness and cost. Without comprehensive data, it is challenging to form accurate conclusions about a subject's health, for example to determine whether a subject has a particular condition or whether a particular therapy will be effective.

Beyond these clinical and logistical sources of missing data, multimodal biomedical data presents technical challenges for computational analysis. Different modalities differ substantially in dimensionality, in native data type (e.g., continuously-valued, categorical or discrete, sequential, spatial, etc.), in noise characteristics, in sparsity, and in the assay-specific and batch-specific artifacts they contain. Conventional computational pipelines for multimodal biomedical analysis generally are not designed to operate on arbitrary subsets of modalities, do not learn the relationships between modalities in a way that supports inference of one modality from another, and do not preserve the fine-grained structure of each modality in a form from which a missing modality could be reconstructed.

Accordingly, conventional computational techniques do not adequately account for missing modalities. They simply combine the available modalities in a downstream task such as classification, and when a modality is missing, results are inaccurate, inconclusive, or the computational techniques are rendered inapplicable. Such conventional techniques often require strict pairing of modalities (e.g., paired tissue image and gene expression data), so an analysis is inconclusive or cannot be performed if one of those modalities is missing. And even when not inconclusive, results are inaccurate because the underlying biological profile is not fully accounted for. Conventional computational pipelines that impute incomplete data using simple statistical techniques do not accurately represent the underlying biology because they do not account for the complex relationships among modalities; they do not learn a model of the cross-modal data-generating process from which a missing modality could be generated in its native format. Downstream tasks that rely on such imputed data therefore produce inaccurate results.

A further limitation of conventional techniques is that they treat multimodal integration as a feature-concatenation or ensemble problem, combining modality representations as inputs to a downstream classifier or regressor without modeling the biological relationships among modalities. Such techniques do not learn how one modality informs or constrains another, for example how a subject's transcriptomic profile relates to morphological features visible in a tissue image, or how blood-derived molecular signals reflect the tissue structure of an inaccessible tumor. Conventional techniques therefore fail to leverage the complementary information contained across modalities, limiting their ability to extract clinically meaningful insights from incomplete datasets, particularly in clinical and translational settings where data is routinely heterogeneous, partially observed, and inconsistent across subjects.

Accordingly, the inventors have developed machine-learning techniques that address these limitations. The techniques are implemented by a multimodal foundation model trained to represent a subject's biology across modalities in a unified computational framework and to generate, reconstruct, or infer data of a target modality from whatever input modalities are available. The multimodal foundation model comprises one or more trained encoders, one or more trained adapters, and one or more trained generative models, which together form the system that performs the disclosed functions.

References herein to “a trained multimodal generative model” refer to one or more trained generative components of the multimodal foundation model that are configured to produce, from one or more adapted representations of input data, an output characterizing biology of the subject in a target modality. In some embodiments, a trained multimodal generative model is a denoising diffusion model, or other model that operates by sampling from a learned distribution, that generates data of a target modality, for example a tissue image or a representation of RNA expression data from which gene expression values can be decoded. In other embodiments, a trained multimodal generative model is a regression model, for example an attention-based multiple instance learning regression model, that generates output values characterizing the target modality, for example signature scores for one or more gene groups that characterize the transcriptomic state of the subject. As used herein, therefore, “generative” refers to the function of producing an output characterizing the target modality from an adapted representation of the input data, and encompasses both sample-generating models operating on a learned distribution over a modality's data space and regression-type models that produce values characterizing the modality, and a trained multimodal generative model may refer to a single such component or to a family of such components, depending on the context.

In some embodiments, the disclosed techniques involve generating (e.g., imputing) data of a target modality from input data of one or more other modalities. The target modality may be a modality for which data is missing for the subject, and the input data is data already available for the subject. In some embodiments, the techniques include: (a) encoding the input data using at least one trained encoder (e.g., a respective trained encoder per input modality) to obtain one or more representations of the input data; (b) processing those representations using at least one trained adapter (e.g., a respective trained adapter per input modality) to obtain one or more adapted representations suitable for subsequent processing by a trained multimodal generative model; and (c) generating data of the target modality by processing the adapted representations using the trained multimodal generative model. The generated data may be used in downstream clinical and analytical tasks, including identifying a treatment for the subject, stratifying the subject for enrollment in a clinical trial cohort, characterizing the tumor microenvironment, and monitoring disease progression, from whatever subset of biological data is available.

The techniques may be implemented using a multi-stage computational architecture comprising three cooperating learned components, which together constitute the multimodal foundation model. The first component is one or more trained encoders, each configured to transform data of a respective input modality from its native representation (for example, a high-dimensional gene expression vector, or a gigapixel whole-slide image) into a compact learned representation, which preserves biologically relevant information from the input data. The second component is one or more trained adapters, each configured to transform an encoder output into a target representation space having a dimensionality and format compatible with the input (conditioning or otherwise) to a trained multimodal generative model. The third component is one or more trained multimodal generative models, each configured to receive one or more adapted representations as input(s) (conditioning or otherwise) and to generate data of a target modality. This three-stage decomposition is itself a technical improvement over prior multimodal analysis pipelines, which typically couple feature extraction and prediction in a single end-to-end model specific to a fixed modality combination. By interposing a dedicated adapter layer between modality-specific encoders and a shared generative backbone, the architecture provides at least the following technical properties: (a) the encoder, adapter, and generative model components may be trained separately or jointly, as appropriate to the training data available for each; (b) pre-trained foundation models, for example self-supervised vision transformers pre-trained on histopathology corpora, or autoencoders pre-trained on RNA-sequencing corpora, may be incorporated as fixed feature extractors within the architecture; (c) new modalities may be incorporated after initial deployment by training only a new encoder-adapter pair, without retraining the generative model; and (d) a single trained generative model may be configured to receive adapted representations corresponding to any subset of available input modalities without architectural modification, because each available modality is provided through the generative model's input interface (whether a conditioning mechanism or other input) and unavailable modalities are simply omitted. These are properties of the architecture, not of any particular downstream analytical task.

The trained encoders and adapters cooperatively produce, for each subject, representations that occupy a shared latent representation space of common dimensionality. This shared space is produced by training the encoders and adapters, and reflects learned cross-modal relationships rather than any predetermined analytical mapping. Within the shared space, measurements of different physical types, for example optical intensities from a stained tissue section, sequence counts from a transcriptomic assay, cell-population frequencies from a cytometry assay, categorical variables and laboratory values from a clinical record, and unstructured text from a clinical note, are placed into a common latent space in which they can be combined using operations such as summation, concatenation, attention-based fusion, and learned gating. The existence of this shared representation is what permits a single generative model to operate on (e.g., be conditioned on) any combination of available input modalities, what permits missing modalities to be reconstructed from those available, and what permits a controlled perturbation applied to one modality to propagate computationally into predicted changes in another. Conventional pipelines generally lack any comparable shared representation, which is why they must be rebuilt for each new modality combination and generally do not support cross-modal inference.

In some embodiments, the multimodal foundation model is trained in a manner that is itself a technical improvement over conventional supervised training of biomedical predictors. Rather than requiring fully paired multimodal training examples, the model may be trained under deliberate partial-observation conditions in which one or more input modalities, features, tokens, or individual measurements are randomly masked, dropped, and/or corrupted, and the generative components are optimized to reconstruct the withheld information from whatever remains observed. Because the training signal is generated by withholding measurements from the training data itself, the regime does not require externally-annotated labels, and can therefore leverage the large volumes of unlabeled and partially-labeled biomedical data available. Because the set of withheld modalities varies across training examples, a single trained model learns, as a byproduct of training, a family of conditional generation functions, one for each possible subset of available input modalities, without requiring a separate model per input-output combination. Conventional supervised approaches, by contrast, generally require a separately-trained model for each fixed input-output pairing, do not produce inferred representations of unobserved modalities, and generally do not exploit unlabeled or partially-paired data to the same extent.

A further consequence of operating in a shared learned latent space, rather than by fitting a direct supervised mapping from inputs to labels, is that the disclosed techniques support computational operations that are not available in conventional biomedical predictors. Specifically, a controlled perturbation may be applied to the input data, for example by modulating values of a selected gene subset in an input expression vector. The generative model may then be re-run to produce the target modality output corresponding to the perturbed state. Because the generative model may be trained to produce outputs consistent with the learned joint distribution across modalities, the difference between outputs for the original and perturbed inputs represents the model's learned estimate of how a change in one compartment of the subject's biology propagates into other compartments. This capability, namely controlled perturbation of input data followed by model-driven generation of the consequent multimodal state, enables in silico exploration of therapeutic response, resistance programs, disease trajectories, and genotype-phenotype relationships, and supports applications such as virtual hypothesis testing of therapeutic agents, discovery of imaging biomarkers associated with specific molecular states, and simulation of disease progression over time. Conventional predictive models, which are generally restricted to producing a single output for a single fixed input, are not designed to perform these operations.

The techniques described herein accordingly provide improvements in the design and operation of multimodal machine-learning systems for biomedical data. These improvements include, without limitation: (1) a modular encoder-adapter-generative-model architecture that decouples modality-specific feature extraction from cross-modal generative reasoning, and permits each component to be trained, replaced, and extended independently; (2) a shared learned latent representation space that places heterogeneous biomedical measurements into a common representation space in which they can be combined, and in which missing modalities can be reconstructed from those present; (3) a training regime based on masked reconstruction from partial observations, which permits a single trained model to handle varying subsets of available input modalities and to be trained on unlabeled and partially-paired data; (4) the ability to incorporate pre-trained foundation models as fixed feature extractors within the architecture, and the ability to incorporate additional modalities after deployment by training only a new encoder-adapter pair; (5) the ability to apply controlled perturbations to input data and generate, using the trained multimodal generative model, the resulting multimodal state, supporting computational operations such as in silico exploration of therapeutic response and disease trajectories; and (6) downstream analytical accuracy measurably closer to ground-truth measurements on held-out data than that produced by conventional approaches. These improvements are improvements to the underlying machine-learning technology and to the computational processing of heterogeneous multimodal biomedical data, and are realized in the specific encoder, adapter, and generative-model architectures, training procedures, and inference procedures described in the sections that follow.

The architecture described herein is extensible by design. Although certain embodiments are described in the context of specific input and target modalities, including RNA expression data, tissue image data, cell-free RNA data, cytometry data, immune repertoire data, and structured and unstructured clinical data, the architecture is not limited to these modalities. Additional modalities, including without limitation spatial transcriptomics, immunohistochemistry, multiplexed spatial imaging, radiological imaging, methylation and other epigenomic assays, proteomic and metabolomic assays, microbiome assays, wearable-sensor data streams, and future biomedical measurement technologies, may be incorporated by training a modality-specific encoder and corresponding adapter that project the new modality into the shared latent representation space used by the trained multimodal generative model. Because the generative model is conditioned through the adapter layer rather than through a fixed input vector of predetermined structure, incorporating a new modality may be accomplished without wholesale retraining or architectural reconfiguration of the generative model itself (e.g., either without re-training, or with fine-tuning).

Following below are descriptions of various concepts related to, and embodiments of, techniques for using a multimodal generative model to generate data of a target modality. Various aspects described herein may be implemented in any of numerous ways, and the techniques are not limited to any particular manner of implementation. Example details of implementations are provided for illustrative purposes only, and the techniques disclosed herein may be used individually or in any suitable combination.

### Multimodal Data Generation Techniques

Generating Data of a Target Modality from Input Data of One or More Modalities

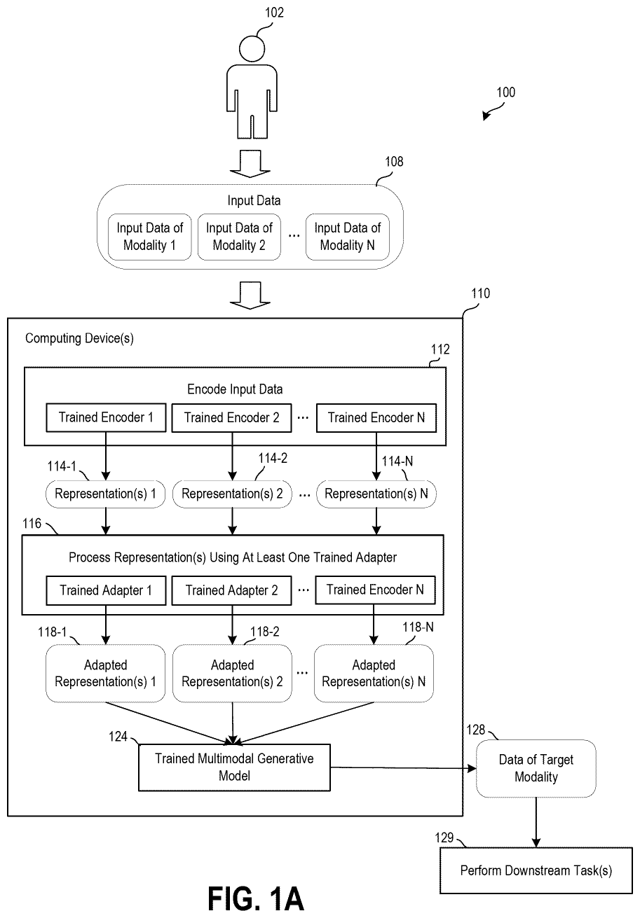

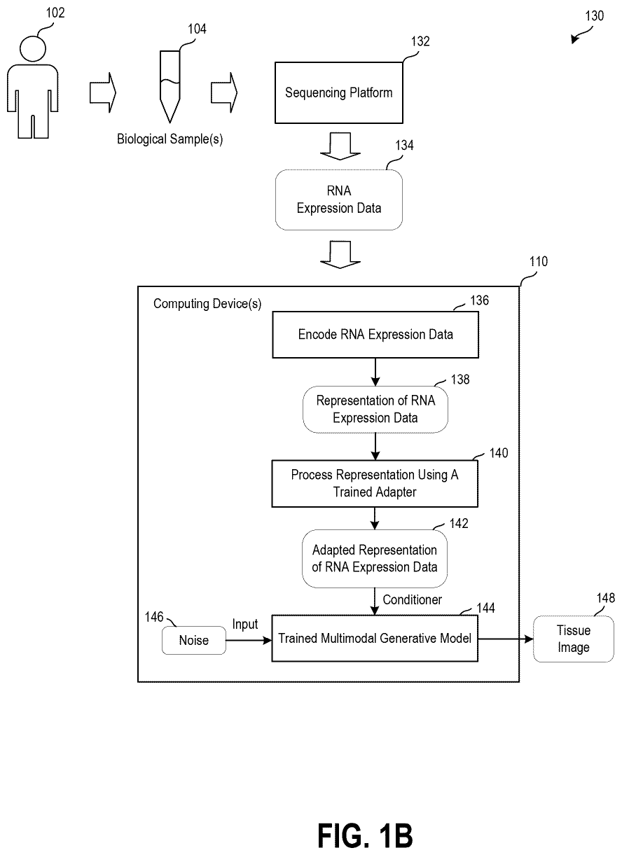

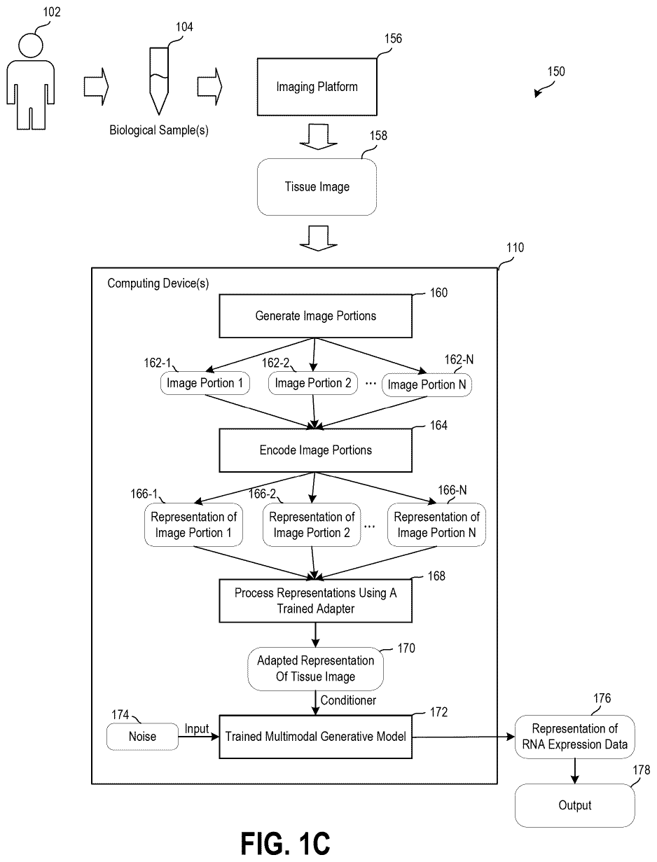

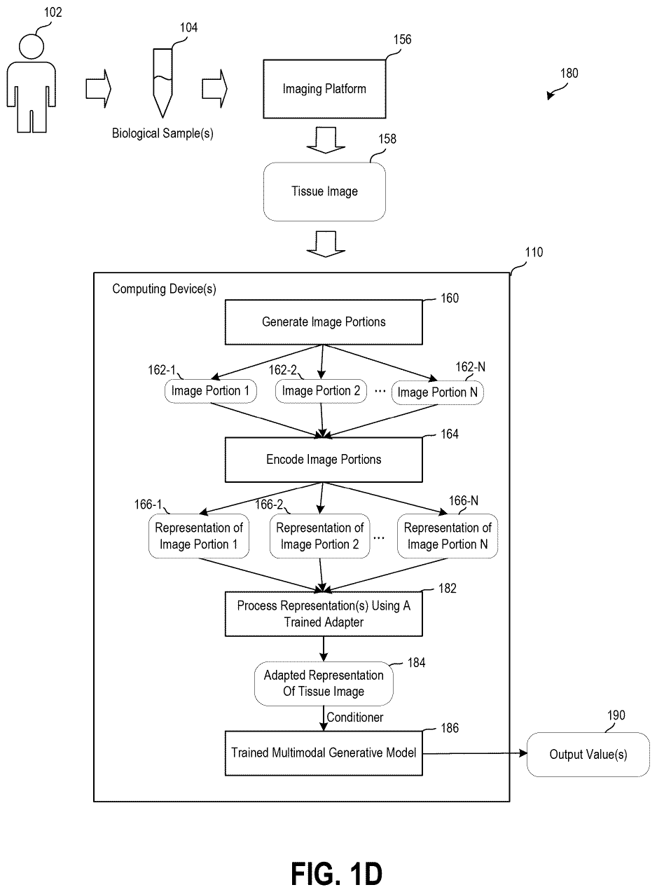

FIG. 1A is a diagram of an illustrative technique for generating data of a target modality, according to some embodiments of the technology described herein. As shown in FIG. 1A, illustrative technique 100 may include processing input data 108, comprising input data of one or more modalities (e.g., modalities 1-N), using computing device(s) 110 to generate data of a target modality 128. Processing the input data 108 may involve: (a) encoding the input data, at act 112, using at least one trained encoder (e.g., trained encoders 1-N) to obtain representation(s) 114-1-114-N of the input data 108 (e.g., one for each input data modality), (b) processing the representation(s) 114-1-114-N of the input data 108 using at least one trained adapter (e.g., trained adapters 1-N) to obtain adapted representation(s) 118-1-118-N of the input data 108, and (c) generating data 128 of the target modality by processing, using the trained generative multimodal generative model 124, the one or more adapted representations of the input data. In some embodiments, illustrative technique 100 additionally includes using the generated data 128, at act 129, to perform one or more downstream tasks.

The input data 108 may include input data of one or more modalities (e.g., modalities 1-N) excluding the target modality. For example, data of the target modality may be missing for the subject 102, and the techniques described herein may be used to generate that missing data from the available input data 108. The modalities 1-N may include any suitable sources of biomedical data about the subject 102. For example, modalities 1-N may include genomic data, epigenomic data, transcriptomic data, image data, cytometry data, spatial-omics data, microbiome data, proteomic data, metabolomic data, cell count data, single-cell omics data, liquid biopsy data, immune cell repertoire data, and clinical data, among others. It should be appreciated that these categories may encompass more specific types of data. For example, transcriptomic data may include RNA expression data (e.g., RNA-sequencing data), and image data may include tissue image data (e.g., hematoxylin and eosin (H&E) stained histopathological images). Additional examples of data types encompassed by these modality categories are listed in Table 1. However, it should be appreciated that the modalities 1-N may include any suitable modality that may be obtained for the subject 102, as aspects of the technology described herein are not limited to any particular modality or combination of modalities.

Input data of a number of modalities, N, may be available for the subject 102. The number of modalities, N, may be 1 modality, at least 2 modalities, at least 3 modalities, at least 4 modalities, at least 5 modalities, at least 10 modalities, between 1 and 20 modalities, or any other suitable number of modalities, as aspects of the technology described herein are not limited in this respect. Different modalities may be available for different subjects. For example, tissue image data may be available for a first subject but not a second subject, while RNA expression data is available for the second subject but not the first subject. Not all modalities need be available for a given subject. The techniques described herein are designed to operate on whatever subset of modalities is available, regardless of which specific modalities are present or absent.

The input data 108 may be obtained from a single biological sample or multiple biological samples. When the input data 108 includes input data of multiple modalities, the different modalities may be derived from multiple different biological samples (e.g., biological samples of different types). For example, a tissue image may be derived from a tissue biopsy, while RNA expression data may be derived from a blood sample. Additionally or alternatively, when the input data 108 includes input data acquired at different times or during different clinical encounters, the input data may be derived from different biological samples collected at different times or during different clinical encounters.

The input data 108 may be obtained or may have been previously obtained by processing one or more biological samples from the subject 102 using one or more data measurement platforms. The data measurement platform(s) used to process a biological sample depends on the type of modality being obtained. For example, a sequencing platform may be used to obtain RNA expression data or genomic data, an imaging platform may be used to obtain tissue image data, and a cytometry platform may be used to obtain cytometry data. Examples of sample processing platforms are listed in Table 1. In some embodiments, the input data 108 has been previously obtained for the subject 102 and is retrieved from a data store for processing by computing device(s) 110.

Additionally or alternatively, the input data 108 may be obtained or may have been previously obtained directly from the subject 102. For example, medical imaging data (e.g., computed tomography (CT), magnetic resonance imaging (MRI), or positron emission tomography (PET) scan data) may be acquired by imaging the subject directly using a clinical imaging system. Radiomic features may be extracted computationally from such medical images. In some embodiments, the input data 108 obtained directly from the subject 102 has been previously acquired as part of routine clinical care and is retrieved from a medical imaging archive or data store for processing by computing device(s) 110.

Additionally or alternatively, the input data 108 may be obtained or may have been previously obtained from clinical records associated with the subject 102. For example, tabular clinical metadata (e.g., age, sex, performance status, prior treatment history, and laboratory values) may be obtained from an electronic health record system. Textual clinical data, such as data from medical notes, pathology reports, or clinical trial records, may similarly be obtained from clinical information systems. In some embodiments, such clinical data has been previously recorded as part of routine clinical care and is retrieved from an electronic health record system or other clinical data store for processing by computing device(s) 110. Examples of systems from which clinical data may be obtained are listed in Table 1.

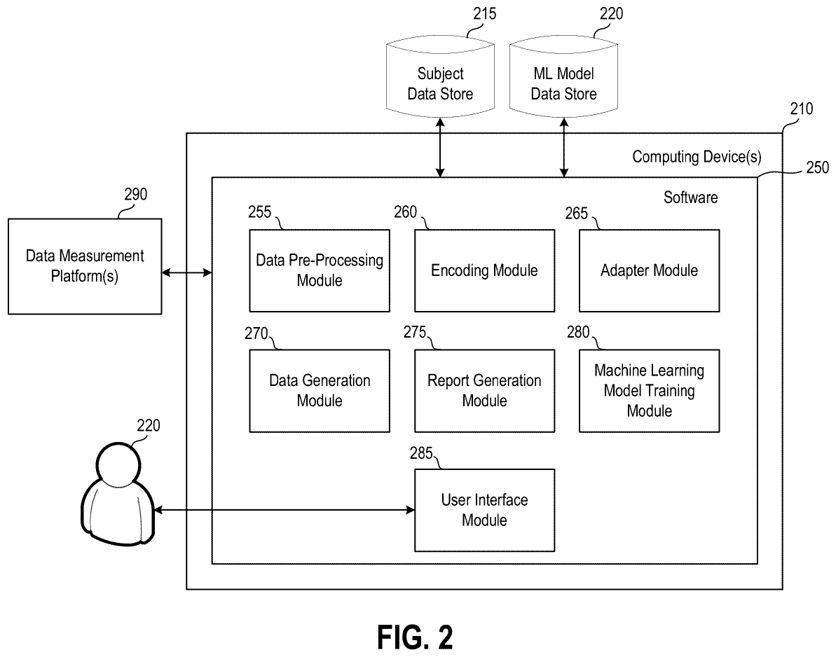

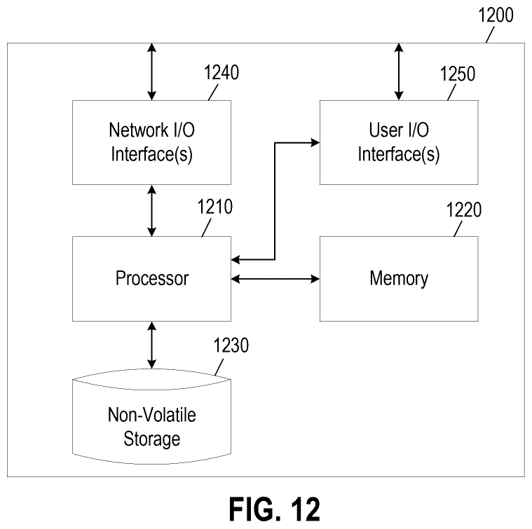

The computing device(s) 110 may include any suitable type of computing device or combination of computing devices, such as one or more servers, laptops, desktops, smartphones, tablets, cloud instances, or virtual machines. Examples of suitable computing devices are described herein with respect to FIG. 2 and FIG. 12. The computing device(s) 110 may comprise a single computing device or multiple computing devices. When multiple computing devices are used, the computing devices may be configured to communicate with one another via one or more communication networks (e.g., the Internet, a local area network, or any other suitable network), and may be part of a distributed or cloud computing environment. In some embodiments, the encoding, adapter processing, and generative model inference steps described herein are performed on hardware accelerators such as graphics processing units (GPUs) or tensor processing units (TPUs). It should be appreciated that aspects of the technology described herein are not limited to any particular type or configuration of computing device.

Software (e.g., software 250 shown in FIG. 2) executing on computing device(s) 110 may be configured to process the input data 108 to generate the data 128 of the target modality. The processing may include: (a) encoding at least some (e.g., all) of the input data to obtain representations 114-1-114-N of the input data 108 (act 112), (b) processing the representations 114-1-114-N using at least one trained adapter to obtain the adapted representations 118-1-118-N of the input data 108, and (c) processing the adapted representations 118-1-118-N using the trained multimodal generative model 124 to obtain the data 128 of the target modality. Aspects of each of these acts are described in more detail herein.

In some embodiments, the input data 108 may be preprocessed prior to being encoded, at act 112. Preprocessing may vary depending on the type of modality and the requirements of the trained encoder. Preprocessing may include one or more of normalization, filtering, segmentation, tiling, augmentation, tokenization, and/or feature extraction, among other operations. For example, preprocessing may involve transforming raw measurements into a standardized representation suitable for encoding, removing low-quality or uninformative data, and/or augmenting the data to improve the robustness of the trained encoder. Examples of preprocessing steps that may be applied to specific modalities are described herein including at least with respect to the section entitled “Multimodal Foundation Model Examples and Applications.” It should be appreciated that aspects of the technology described herein are not limited to any particular preprocessing approach, and that the specific preprocessing steps applied to the input data 108 may vary depending on the modality, the source of the data, and the requirements of the downstream encoding and generation steps.

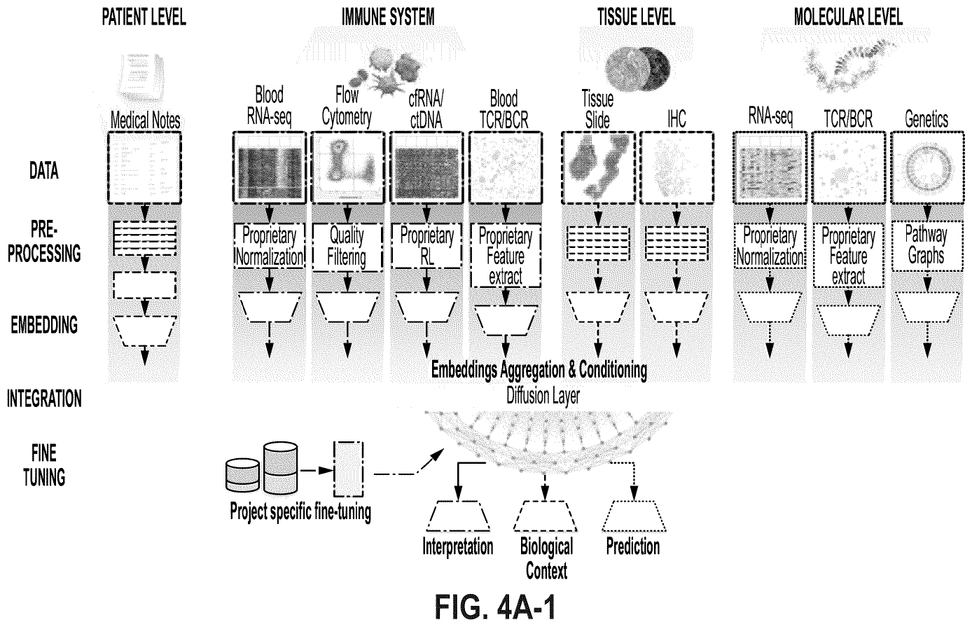

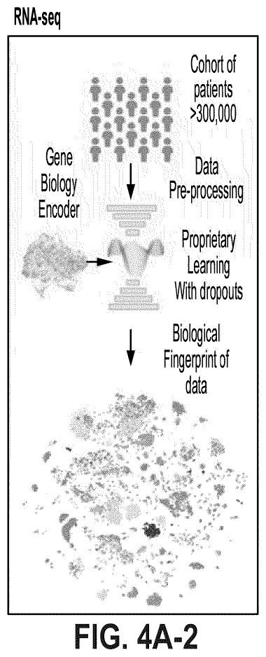

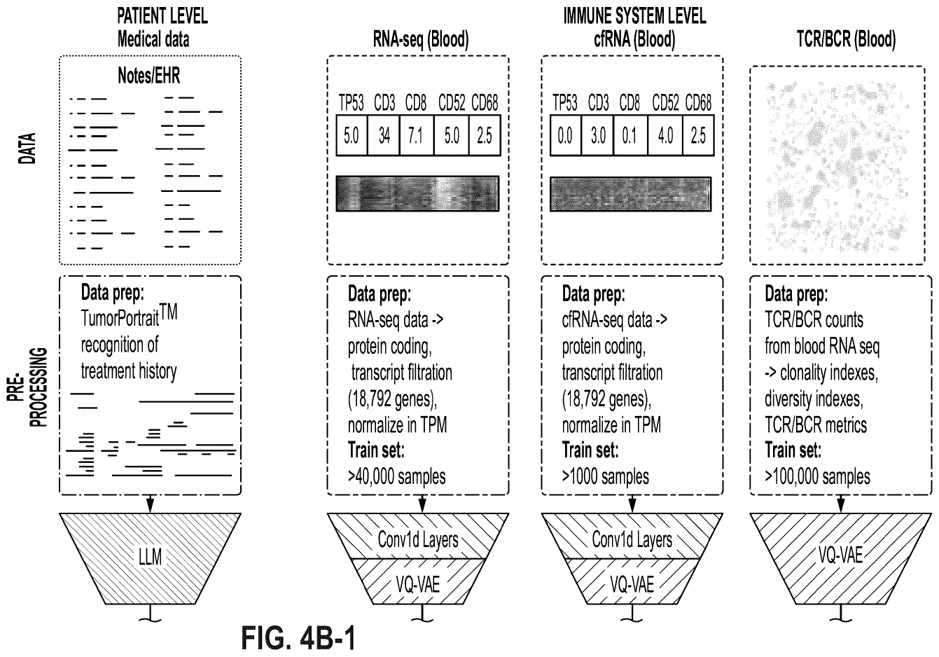

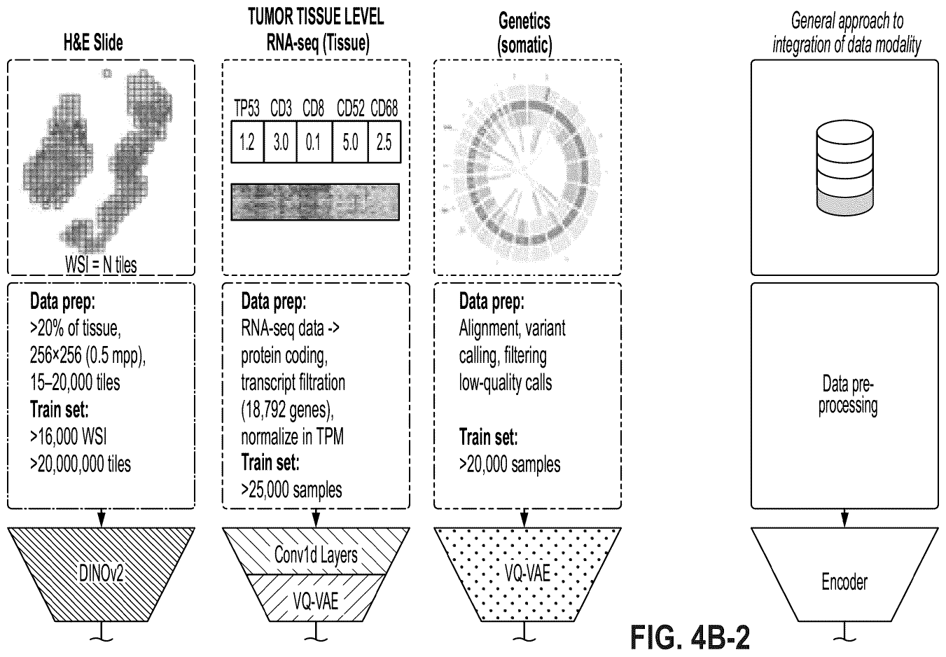

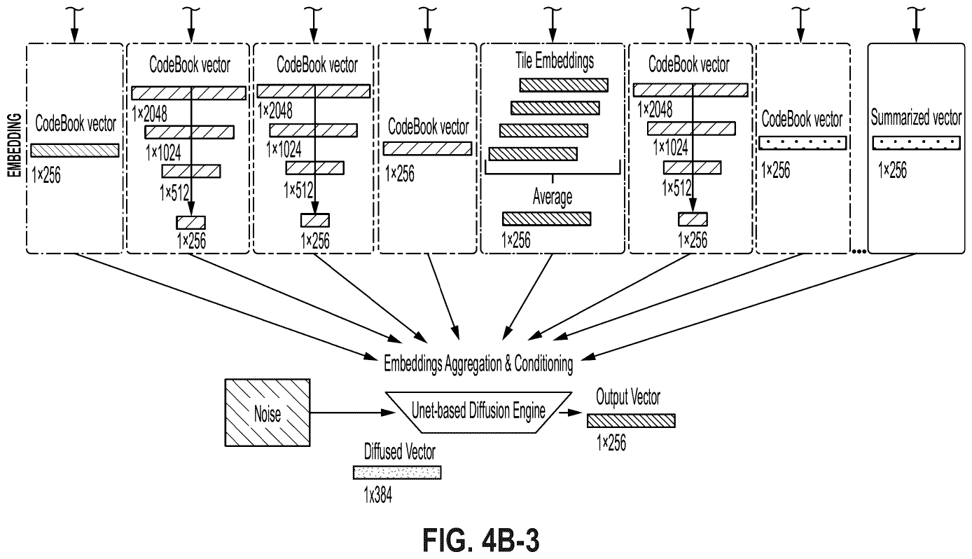

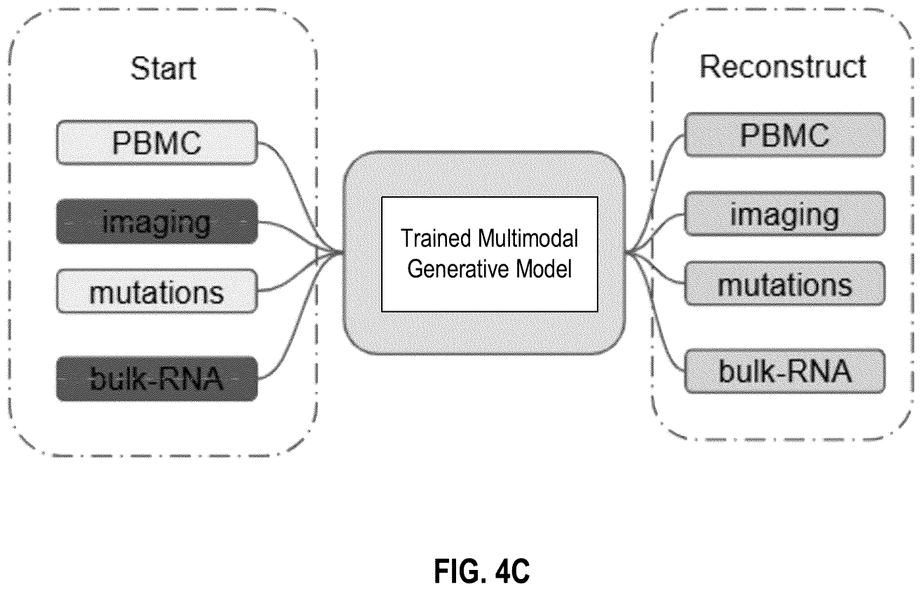

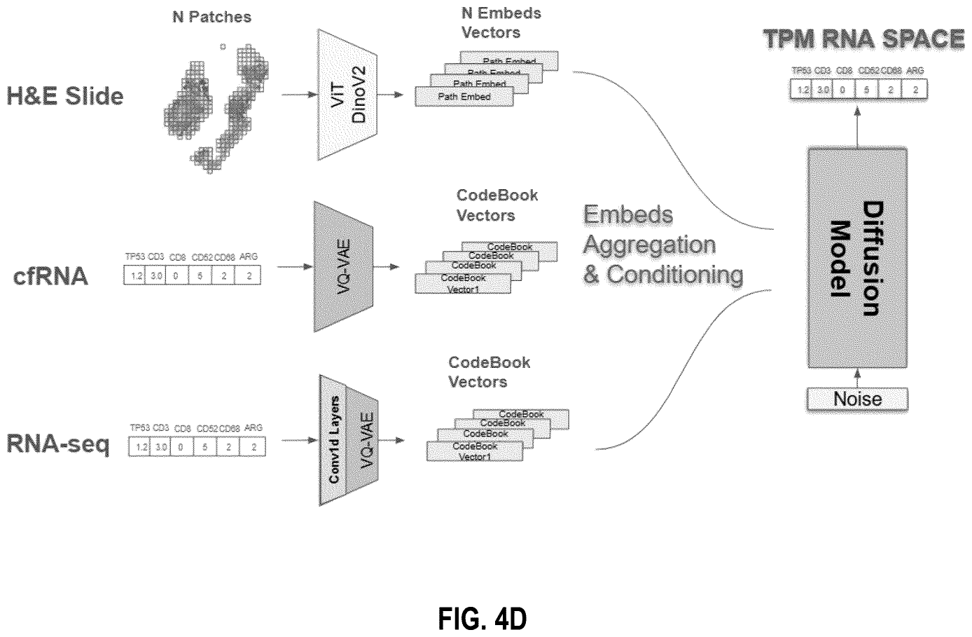

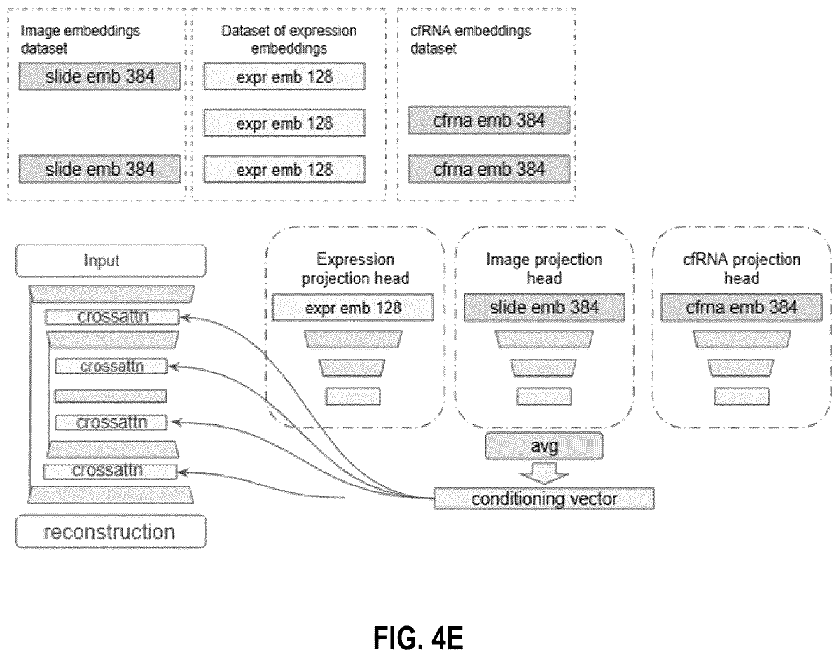

As shown in FIG. 1A, processing the input data 108 (e.g., preprocessed input data) includes encoding at least some of the input data 108, at act 112, using at least one trained encoder to obtain one or more representations 114-1-114-N of the input data 108. In some embodiments, a respective trained encoder is used to encode the input data of each modality. For example, a first trained encoder may be used to encode input data of modality 1 to obtain representation 114-1, a second trained encoder may be used to encode input data of modality 2 to obtain representation 114-2, and so on. FIG. 4D is an example illustrating that a respective trained encoder may be used to encode input data of each of multiple modalities. For example, as shown in FIG. 4D, a vision transformer (ViT) is used to encode H&E images, while respective vector-quantized variational autoencoders (VQ-VAEs) are used to encode cfRNA and RNA-seq data. In some embodiments, a single trained encoder may be used to encode input data of multiple modalities.

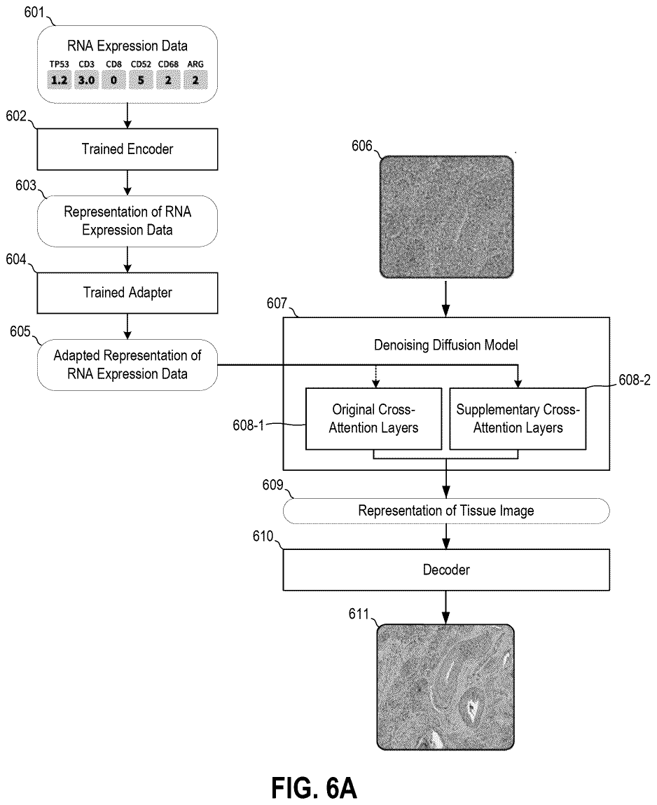

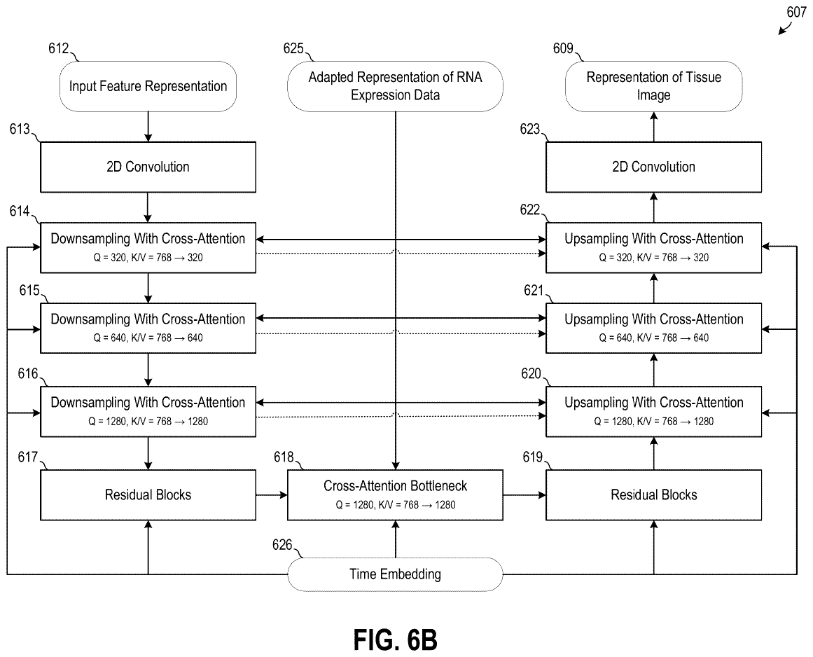

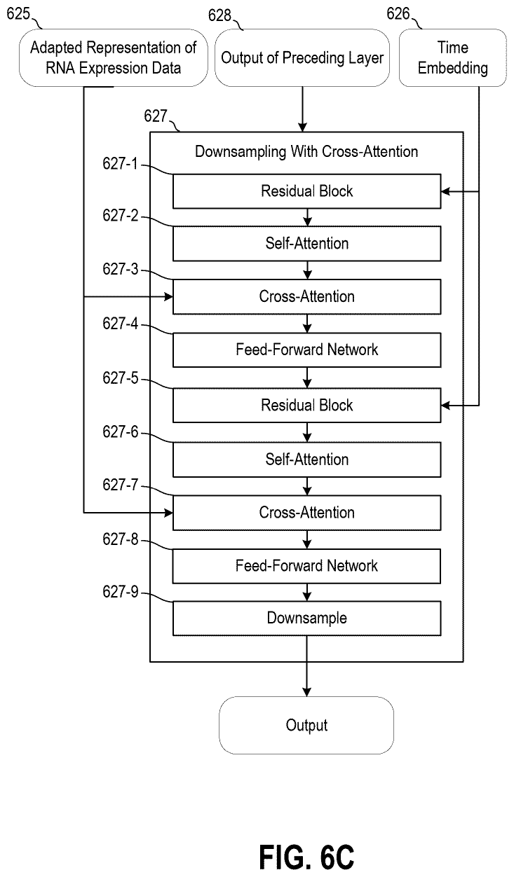

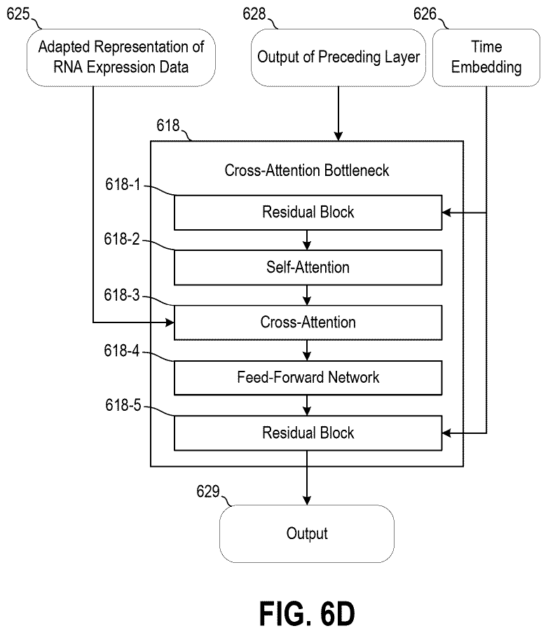

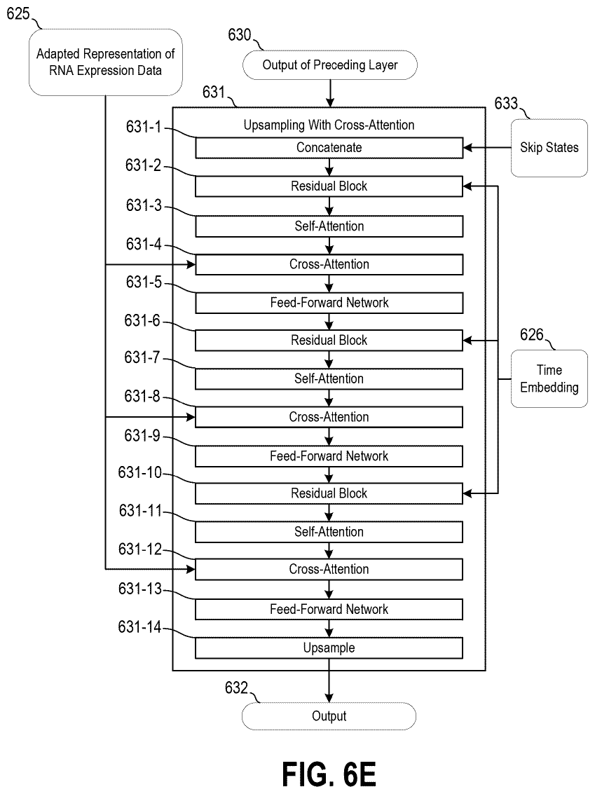

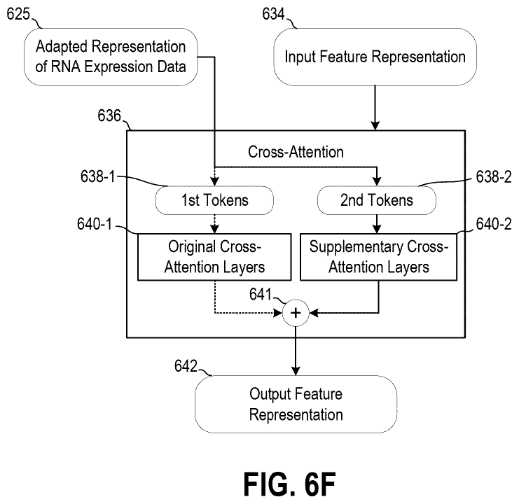

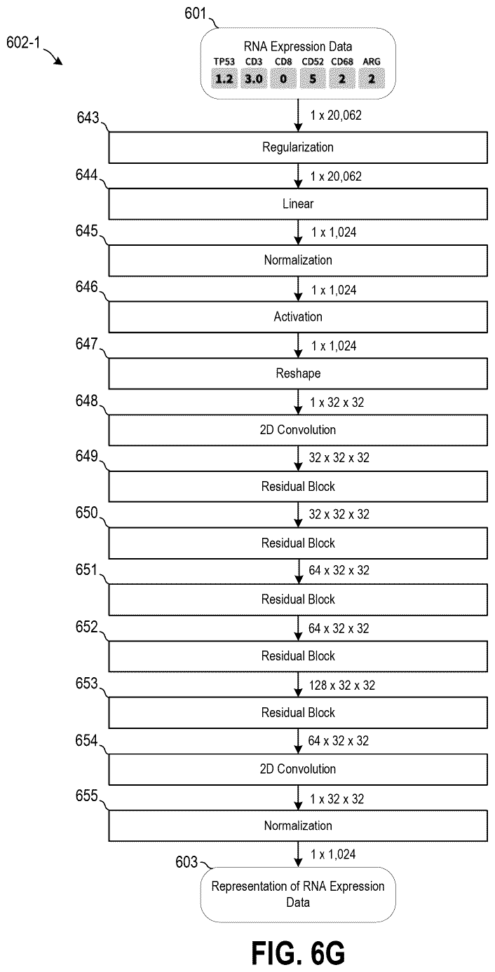

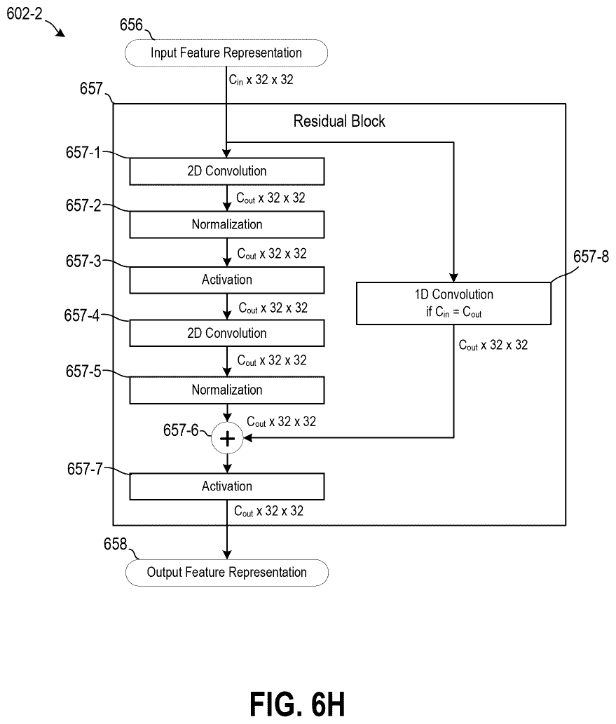

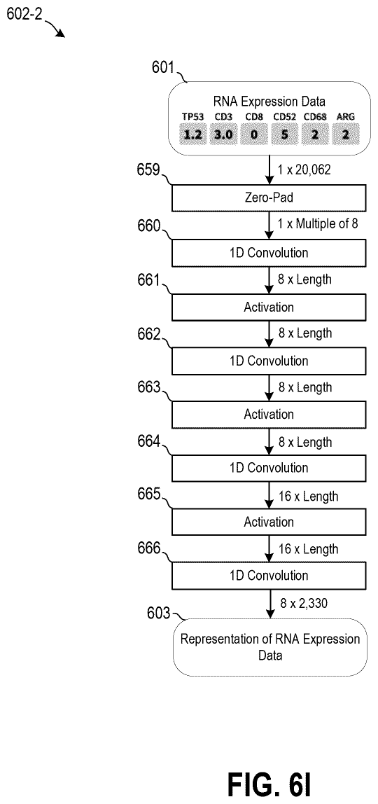

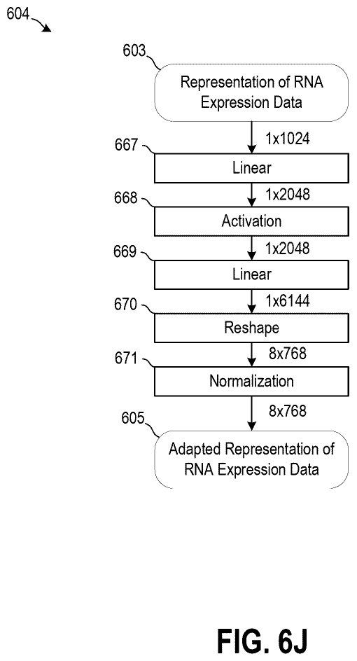

A trained encoder may be any suitable type of encoder capable of encoding input data of the relevant modality into a numeric representation. The type of encoder used may depend on the type and structure of the modality being encoded. For example, the specific encoder architecture used for a given modality may be selected based on the nature of the modality, the availability of training data, and the desired properties of the resulting representation. Examples of types of encoders that may be used to encode input data include autoencoders (e.g., VAE, VQ-VAE), masked autoencoder, latent image autoencoder (LIAE), etc.), transformers (e.g., ViT), convolutional neural networks (CNNs), graph neural networks, multilayer perceptrons (MLPs), embedding tables, recurrent neural networks (RNNs), or any other suitable type of encoder, as aspects of the technology described herein are not limited in this respect. For example, RNA expression data may be encoded using an autoencoder. Tissue image data may be encoded using a transformer. VAEs are described by Kingma, D. P., and Welling, M. (“Auto-encoding variational bayes.” ar Xiv preprint arXiv: 1312.6114 (2013)); VQ-VAEs are described by Van Den Oord, A. and Vinyals, O. (“Neural discrete representation learning.” Advances in neural information processing systems 30 (2017)); and ViTs are described by Dosovitskiy, A., et al. (“An image is worth 16×16 words: Transformers for image recognition at scale.” ar Xiv preprint arXiv: 2010.11929 (2020)), each of which is incorporated by reference herein in its entirety. Examples of a LIAE are described herein with respect to FIG. 6G-6H. Examples of encoders used for encoding specific types of data modalities are described herein including at least with respect to FIGS. 6A, 6G-6I, 8A-8B, and 9C and in the section entitled “Multimodal Foundation Model Examples and Applications.”

In some embodiments, a trained encoder is a large model pre-trained on a large and diverse corpus of data using self-supervised or other objectives (a “pre-trained foundation model”), which may be used as a fixed feature extractor without further fine-tuning. In some embodiments, a pre-trained foundation model may be fine-tuned on domain-specific data prior to use as an encoder.

The representation(s) 114-1 through 114-N obtained by encoding the input data 108 may take any suitable form. For example, a representation may be a vector, a sequence of tokens, a two-dimensional feature map, or any other suitable numeric representation of the input data, depending on the encoder architecture used. The dimensionality and structure of the representation may be determined by the architecture of the trained encoder and may be configured to capture the biologically relevant features of the input data in a compact form suitable for subsequent processing by the trained adapter. Examples of specific encoder architectures and the representations they produce are described herein including at least with respect to FIGS. 4D, 6G, 6H, 6I, 8B, and 9B-9C.

As shown in FIG. 1A, processing the input data 108 may further include processing the representation(s) 114-1-114-N, at act 116, using at least one trained adapter to obtain one or more adapted representations 118-1-118-N of the input data 108. The trained adapter(s) serve as a bridge between the trained encoder(s) and the trained multimodal generative model 124, transforming the representation(s) produced by the encoder(s) into a form that is compatible with and suitable for use as input to, or for conditioning, the trained multimodal generative model 124. Because the trained encoder(s) and the trained multimodal generative model 124 may have been trained independently and on different data, the representations 114-1-114-N produced by the encoder(s) may not be in a form that the generative model can directly interpret as input or as a conditioning signal. For example, a trained encoder may produce a compact numeric representation of RNA expression data that captures biologically relevant features of the input, but that representation may not be in a dimensionality, format, or latent space that is directly compatible with the conditioning mechanism of a pre-trained denoising diffusion model. The trained adapter addresses this by learning a mapping between the two spaces, transforming the encoder's representation into an adapted representation of the appropriate dimensionality and format for the generative model's conditioning mechanism, while preserving the biological information captured by the encoder. This modular design, in which the encoder, adapter, and generative model are developed and trained as separable components connected via the adapter, provides flexibility in that the encoder, adapter, and generative model components may be selected, replaced, and/or updated independently of one another.

In some embodiments, a respective trained adapter is used to process the representation of each input modality. For example, a first trained adapter may be used to process representation 114-1 to obtain adapted representation 118-1, a second trained adapter may be used to process representation 114-2 to obtain adapted representation 118-2, and so on. In some embodiments, a single trained adapter may be used to process representations of multiple modalities. FIG. 4E is an example illustrating that a respective trained adapter may be used to process the representation of each input modality. For example, as shown in FIG. 4E, an expression projection head is used to process expression embeddings to obtain adapted representations of the expression data, an image projection head is used to process image embeddings to obtain adapted representations of the image data, and a cfRNA head is used to process cfRNA embeddings to obtain adapted representations of the cfRNA data.

A trained adapter may be any learned or fixed transformation that maps the representation(s) 114-1-114-N to a target representation space. For example, the target representation space may be a space having a dimensionality and format suitable for use as input to, or for conditioning, the trained multimodal generative model 124. The trained adapter may comprise one or more linear projection layers, non-linear activation layers, normalization layers, reshaping operations, and/or attention-based aggregation mechanisms, or any combination thereof. In some embodiments, the trained adapter is configured to perform multiple instance learning. In such embodiments, the trained adapter may comprise at least one feature extraction network and/or at least one gated attention network. For example, in embodiments in which the input data comprises a tissue image divided into image portions, the trained adapter may be configured to perform multiple instance learning to aggregate the representations of the image portions into a single adapted representation of the tissue image as a whole using at least one gated attention network and/or at least one feature extraction network. In some embodiments in which the trained multimodal generative model 124 comprises a denoising diffusion model, the trained adapter may be configured to map the representation(s) 114-1-114-N to a sequence of tokens of an embedding dimension compatible with the cross-attention layers of the denoising diffusion model. Examples of trained adapters are described herein including at least with respect to FIGS. 6J, 8C, and 10B.

The adapted representation(s) 118-1-118-N produced by the trained adapter(s) may take any suitable form of a dimensionality and format compatible with the trained multimodal generative model 124. For example, an adapted representation may take the form of a vector, a sequence of tokens, or a two-dimensional feature map, among others.

As shown in FIG. 1A, the trained multimodal generative model 124 processes the adapted representation(s) 118-1-118-N to generate data 128 of the target modality. The trained multimodal generative model 124 may be any trained model that generates an output from the adapted representation(s) 118-1-118-N. In some embodiments, this includes a diffusion-based model, such as a latent diffusion model, that generates data of a target modality by iteratively denoising a noised input conditioned on the adapted representation(s) 118-1-118-N. In some embodiments, this includes models that output one or more output values for the subject from the adapted representation(s) 118-1-118-N. For example, the trained multimodal generative model 124 may include a regression model trained to output one or more values (e.g., gene group signature scores) for the subject. In such embodiments, the adapted representation(s) 118-1-118-N may be provided as input to the model, rather than used for conditioning a noise input.

In some embodiments, the adapted representation(s) 118-1-118-N are provided to the trained multimodal generative model 124 separately. For example, the adapted representation(s) 118-1-118-N may be provided to the model separately via dedicated input channels. For example, when the trained multimodal generative model 124 is a denoising diffusion model, the adapted representation(s) 118-1-118-N may be provided as input through dedicated cross-attention channels of the denoising diffusion model. In some embodiments, the adapted representations 118-1-118-N are first combined into one or more unified representations (e.g., a single unified patient-level representation), and the unified representation(s) are provided to the trained multimodal generative model 124. For example, adapted representations may be combined by averaging, concatenation, gating, attention-based fusion, or using any other suitable techniques for combining the adapted representation(s) 118-1-118-N, as aspects of the technology described herein are not limited in this respect. For example, as shown in FIG. 4D, the adapted representations of the expression data, image data, and cfRNA data are combined by averaging to obtain the conditioning vector.

As described herein, in some embodiments, the trained multimodal generative model 124 is a denoising diffusion model. A denoising diffusion model may learn to generate data by learning to reverse a forward diffusion process in which noise is progressively added to training data. The neural network backbone of the denoising diffusion model may be a UNet, a transformer, or any other architecture suitable for performing diffusion, as aspects of the technology described herein are not limited in this respect. For example, the neural network backbone may be the Stable Diffusion 1.5 pre-trained backbone. In some embodiments, as described herein, the neural network backbone is conditioned on the adapted representation(s) 118-1-118-N, so that the denoising process is guided by the available input modality data. In some embodiments, the denoising diffusion model operates directly in the data space of the target modality. In other embodiments, the denoising diffusion model operates in a compressed latent space defined by a pre-trained encoder-decoder pair. In such embodiments, the denoising diffusion model may generate a latent representation of the data of the target modality (e.g., a latent representation of a tissue image or RNA expression data) that is subsequently decoded into the target modality (e.g., a tissue image or RNA expression data) by a decoder. Such a model is referred to herein as a latent diffusion model. Latent diffusion models are described by Rombach, R., et al. (“High-resolution image synthesis with latent diffusion models.” Proceedings of the IEEE/CVF conference on computer vision and pattern recognition. 2022.); and the U-Net architecture is described by Ronneberger, O., Fischer, P., and Brox, T. (“U-net: Convolutional networks for biomedical image segmentation.” International Conference on Medical image computing and computer-assisted intervention. Cham: Springer international publishing, 2015.), each of which is incorporated by reference herein in its entirety. Example techniques for training a denoising diffusion model are described herein including at least in the section entitled “Training.”

In some embodiments, at inference time a reverse diffusion process is applied. Starting from a random noise input, the trained neural network may iteratively denoise the input over a series of timesteps, conditioned on the adapted representation(s) 118-1-118-N of the input data, to produce a generated sample of the target modality for inclusion in the data 128. In some embodiments, a guidance scale parameter (e.g., between 1 and 12, such as 7.5) is used at inference to interpolate between the conditional and unconditional model predictions to improve the quality and fidelity of the generated output. In some embodiments, the number of inference steps is reduced relative to the number of training timesteps. For example, the number of inference steps may be reduced using a denoising diffusion implicit model (DDIM) sampling procedure. In alternative embodiments, the DDPM sampling procedure may be used. DDIM is described by Song, J., Meng, C., and Ermon, S. (“Denoising diffusion implicit models.” arXiv preprint arXiv: 2010.02502 (2020)); and DDPM is described by Ho, J., Jain, A., and Abbeel, P. (“Denoising diffusion probabilistic models.” Advances in neural information processing systems 33 (2020): 6840-6851), each of which is incorporated by reference herein in its entirety. In some embodiments, the denoising diffusion model is used to generate multiple samples for inclusion in the data 128 of the target modality. For example, the multiple samples may be generated from the same conditioning input using different random noise inputs.

In some embodiments, the trained multimodal generative model 124 is a regression model. In some embodiments, the regression model takes the adapted representation(s) 118-1-118-N as input and directly maps them to one or more output values in a single forward pass. The regression model may comprise any suitable neural network architecture capable of mapping the adapted representation(s) to the desired output values. For example, the neural network architecture may include one or more linear layers (e.g., fully connected linear layer(s)) that map the adapted representation to the one or more output values (e.g., a vector of one or more continuous output values). In some embodiments, the regression model comprises a regression head that predicts a plurality of output values simultaneously (e.g., predicting signature scores for a plurality of gene groups simultaneously from a single adapted representation of a tissue image). In some embodiments, the regression model comprises a plurality of regression heads (e.g., a respective regression head for each output value) that each independently predict a respective output value from the adapted representation(s) 118-1-118-N, enabling per-output attention and interpretability. Examples of regression models are described herein including at least with respect to FIGS. 1D, 10A, and 11A. Example techniques for training a regression model are described herein including at least in the section entitled “Training.”

As shown in FIG. 1A, the output of the trained multimodal generative model 124 includes data 128 of the target modality. The data 128 of the target modality may take any suitable form depending on the target modality type. The target modality type may include any of the modality described with respect to the input data 108. Though it should be appreciated that the target modality type may be a modality that is not included in the input modality types. For example, the input data 108 may include tissue image data while the data 128 includes RNA expression data, and vice versa. Alternatively, data of the target modality may already be included in the input data, and illustrative technique 100 may be used to generate additional or alternative samples of the target modality.

The data 128 of the target modality may include a single sample or multiple samples. For example, as described herein, a denoising diffusion model may be used to generate multiple samples from the same conditioning input using different random noise inputs. Additionally or alternatively, the data 128 may include one or more representations of data of the target modality. For example, in embodiments in which the trained multimodal generative model 124 operates in a compressed latent space, the data 128 of the target modality may include latent representation(s) generated by the trained multimodal generative model 124 and/or data derived therefrom. For example, a decoder may be used to process the latent representation(s) to obtain data in the original data space of the target modality.

In some embodiments, illustrative technique 100 additionally includes using the generated data 128 of the target modality, at act 129, to perform one or more downstream tasks. The generated data 128 may be used in any suitable downstream task, either alone or in combination with the input data 108 from which it was generated. For example, the generated data 128 may be combined with the available input data 108 to form a more comprehensive multimodal profile for the subject 102, and that comprehensive profile may be used to perform downstream tasks that would otherwise be infeasible or less accurate due to the absence of the target modality data. In this way, the techniques described herein enable downstream tasks to be performed using a complete multimodal profile for the subject, regardless of which modalities were actually measured.

In some embodiments, the generated data 128 and (optionally) the input data 108 is used to identify a treatment for the subject 102. For example, the generated tissue image or RNA expression data may be used to characterize the subject's tumor microenvironment, identify molecular subtypes, and/or predict response to a particular therapeutic agent. The results of such an analysis may be used to identify a treatment that is likely to be effective for the subject. In some embodiments, the identified treatment is subsequently administered to the subject 102.

In some embodiments, the generated data 128 and (optionally) the input data 108 is used to identify the subject 102 as a member of a cohort, such as a clinical trial cohort. For example, the generated data 128 may be used to determine whether the subject satisfies eligibility criteria for enrollment in a clinical trial, without requiring the subject to undergo additional assays that may be invasive, costly, or infeasible. In some embodiments, the subject is enrolled in the clinical trial based at least in part on the generated data 128 and (optionally) the input data 108.

In some embodiments, the generated data 128 and (optionally) the input data 108 is used to stratify the subject 102 (e.g., into a predicted responder or non-responder group) based on features (e.g., molecular features, morphological features, etc.) captured in the generated data 128 and (optionally) the input data 108. Such stratification may be used to inform treatment decisions, trial enrollment, or patient monitoring strategies.

In some embodiments, the generated data 128 and (optionally) the input data 108 is used to characterize the tumor microenvironment of the subject 102. For example, gene expression signature scores for immune cell populations, inflammatory states, stromal activation, metabolic programs, or other tumor microenvironment processes may be derived from generated RNA expression data or predicted directly by the trained multimodal generative model 124. Such characterization may provide clinically meaningful information about the subject's disease state without requiring a molecular assay.

In some embodiments, the generated data 128 is used to monitor disease progression for the subject 102. For example, data of the target modality may be generated at multiple timepoints using input data obtained at those timepoints, and the generated data may be compared across timepoints to identify changes in the subject's biological state over time. This may be particularly useful in longitudinal monitoring settings where repeated tissue biopsies are infeasible, as described herein.

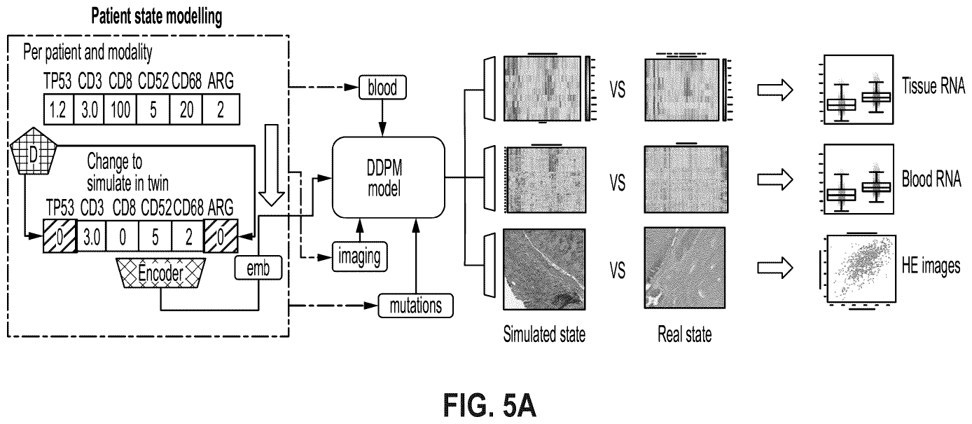

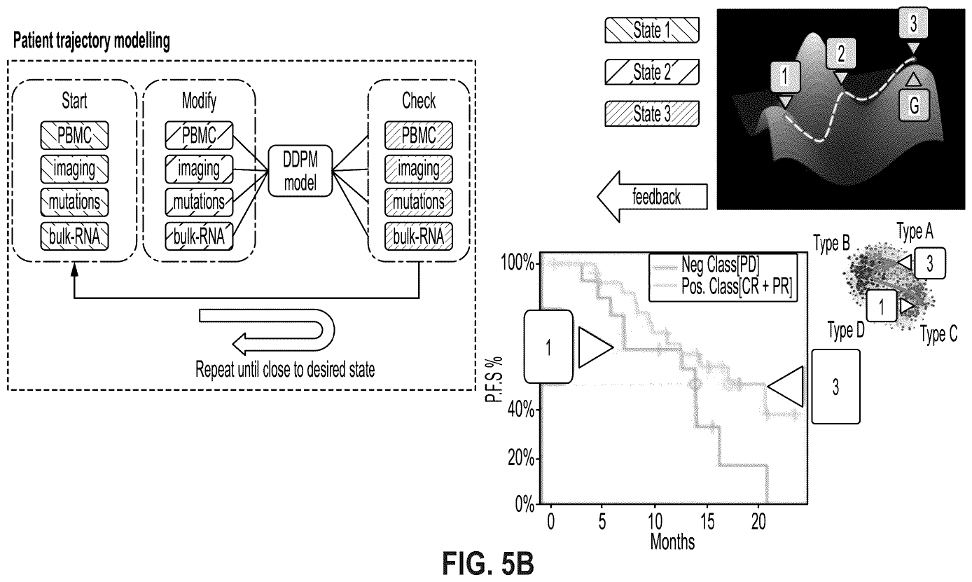

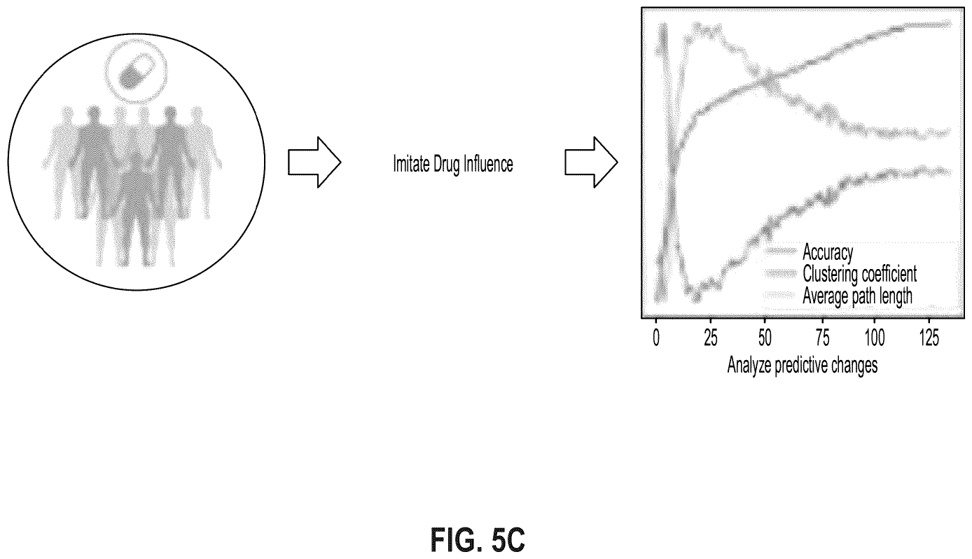

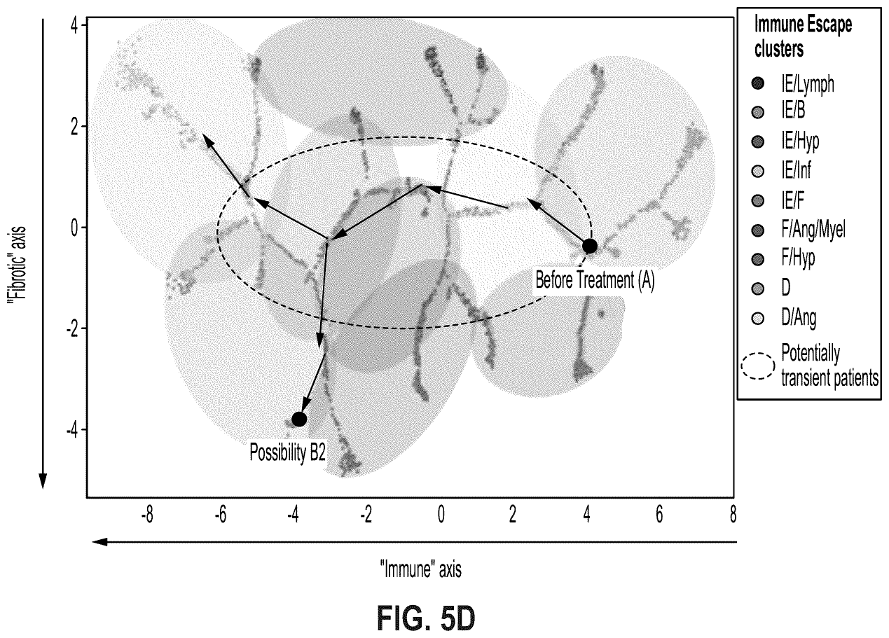

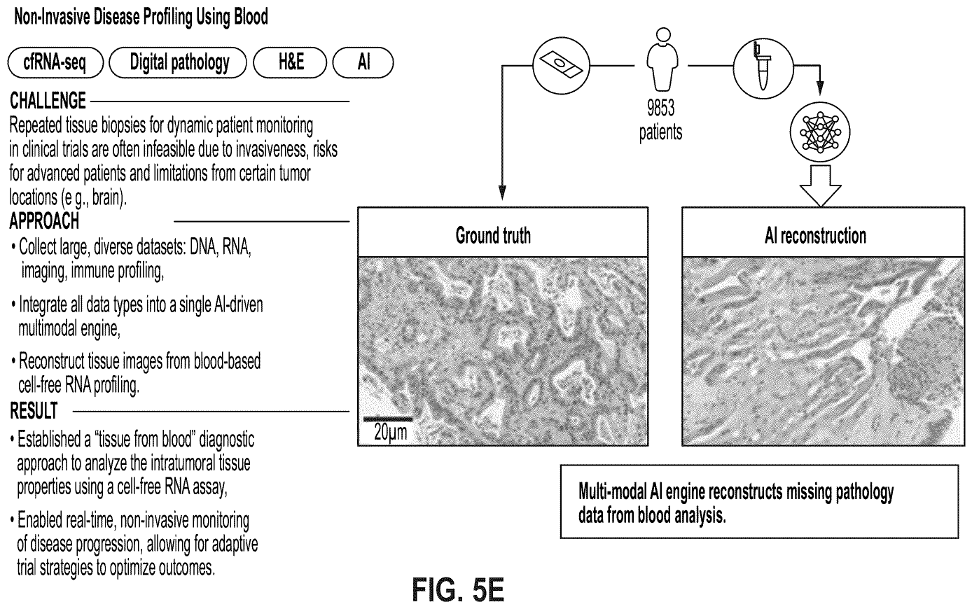

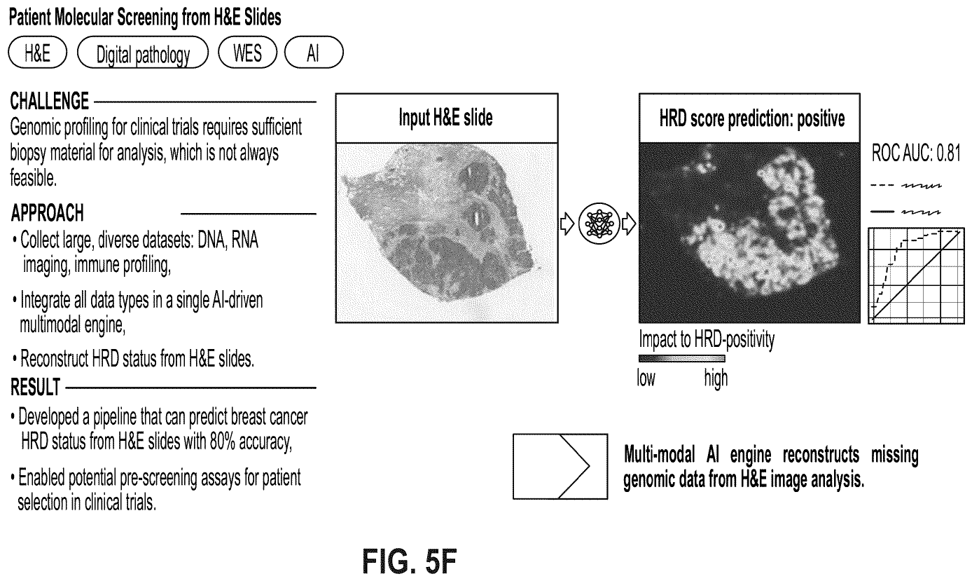

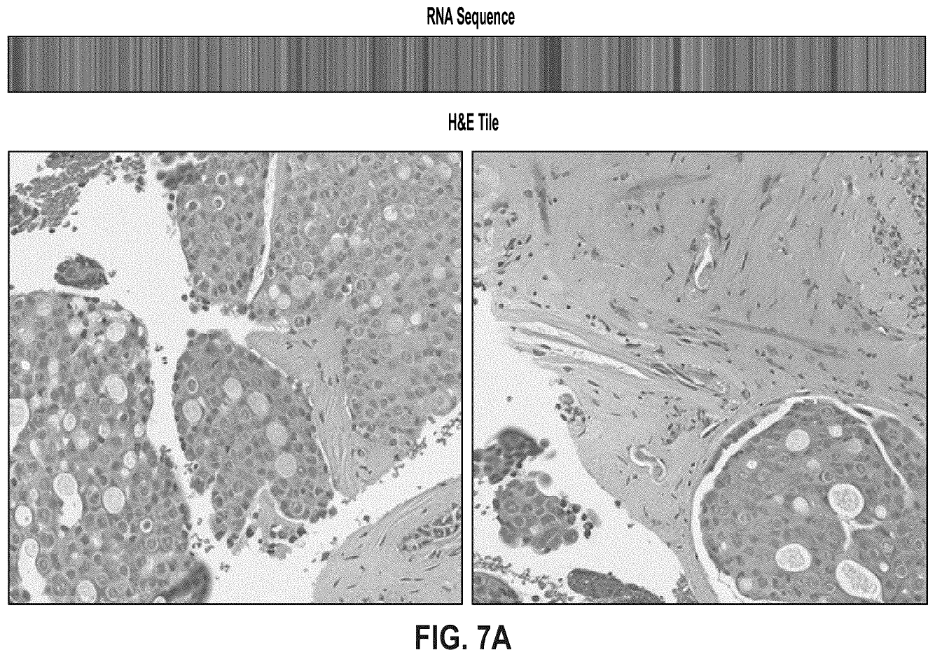

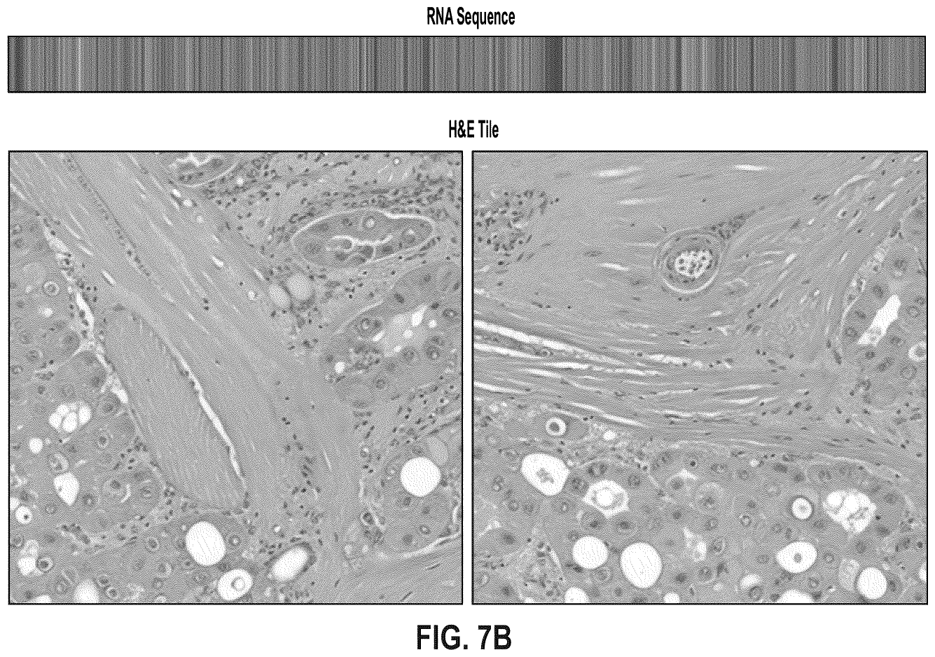

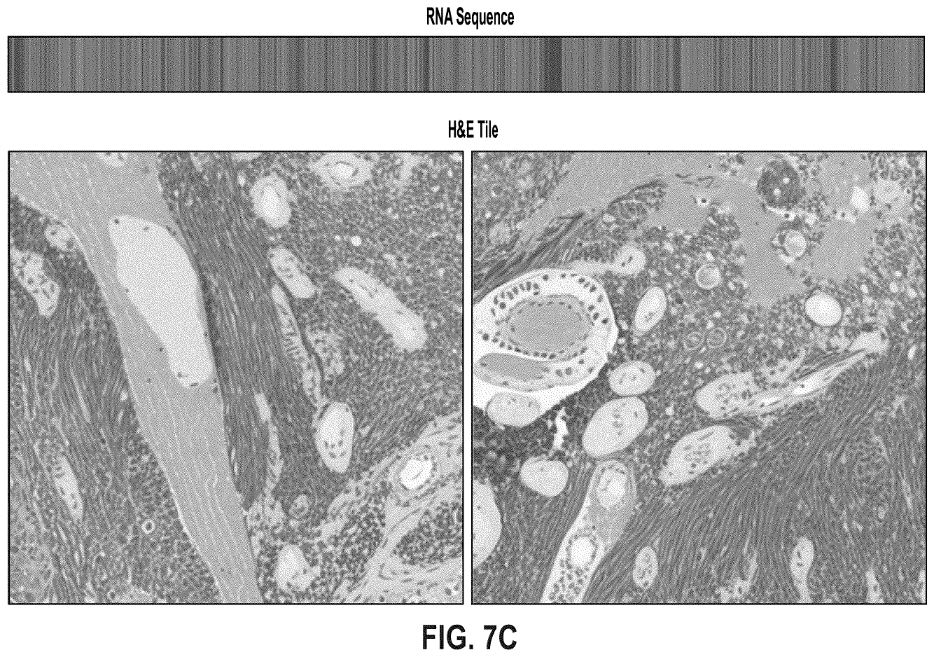

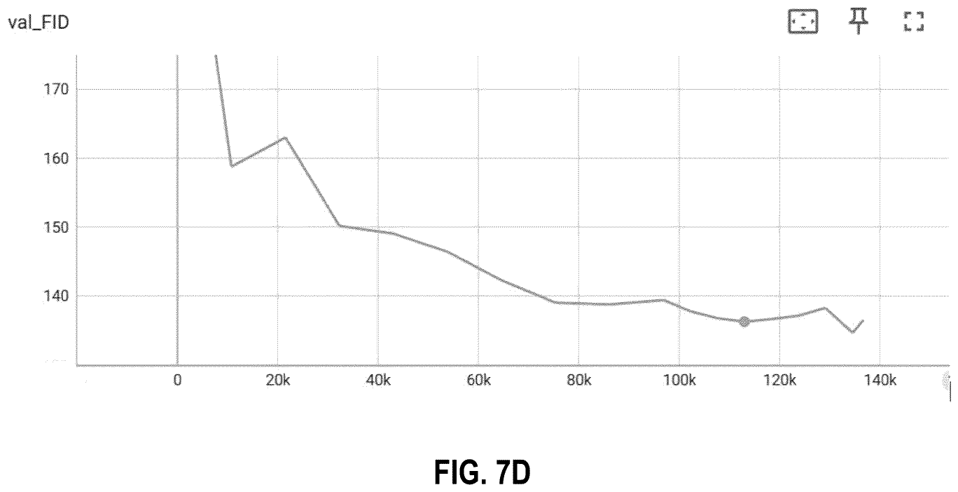

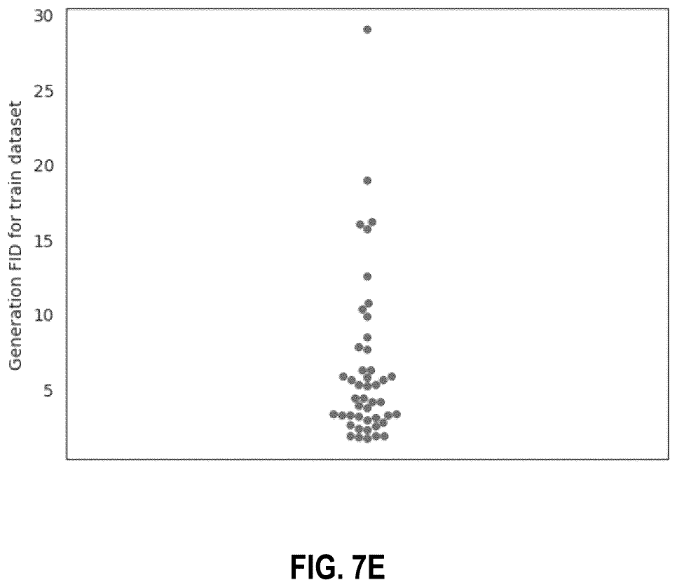

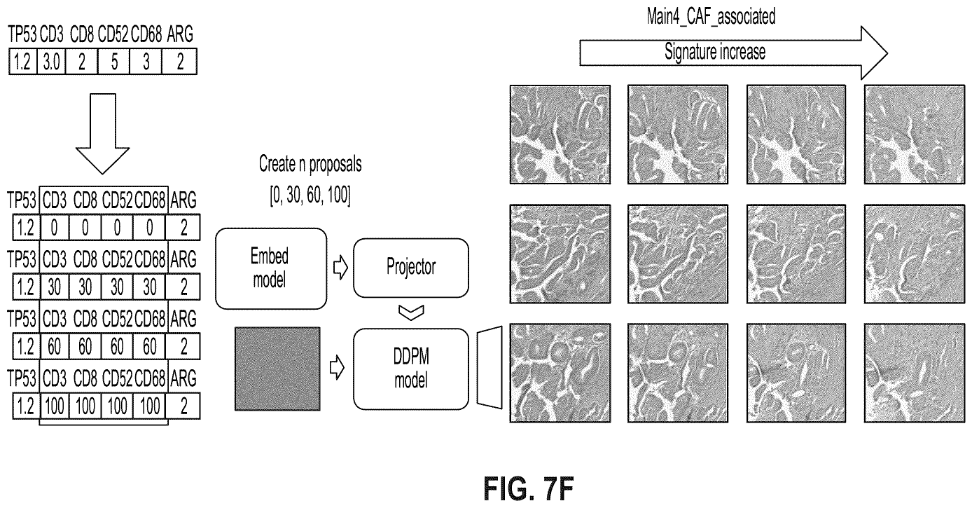

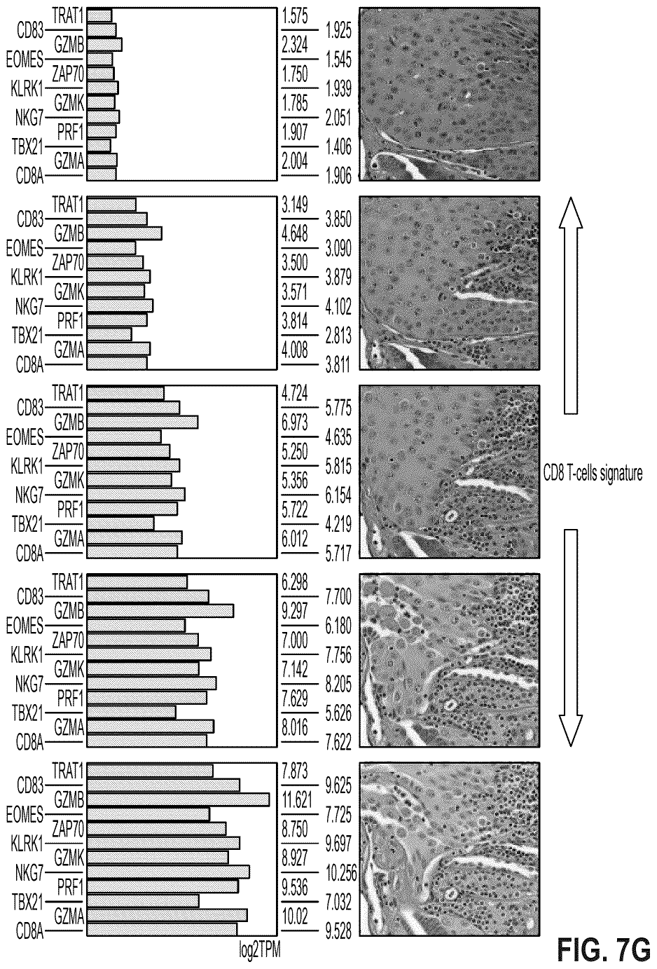

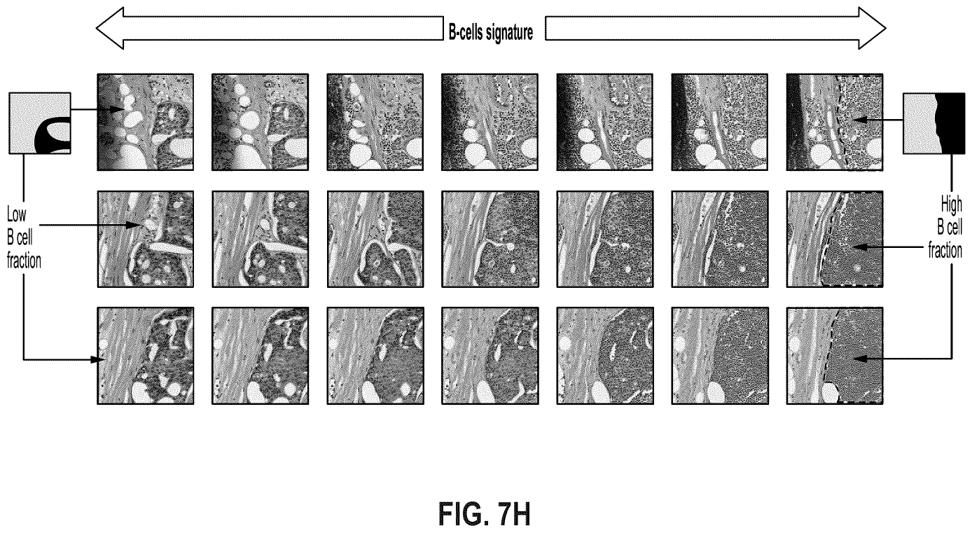

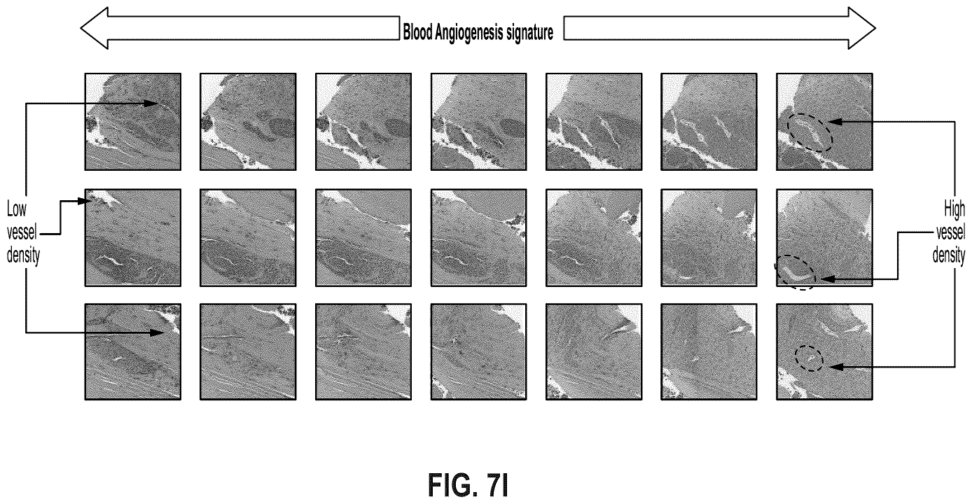

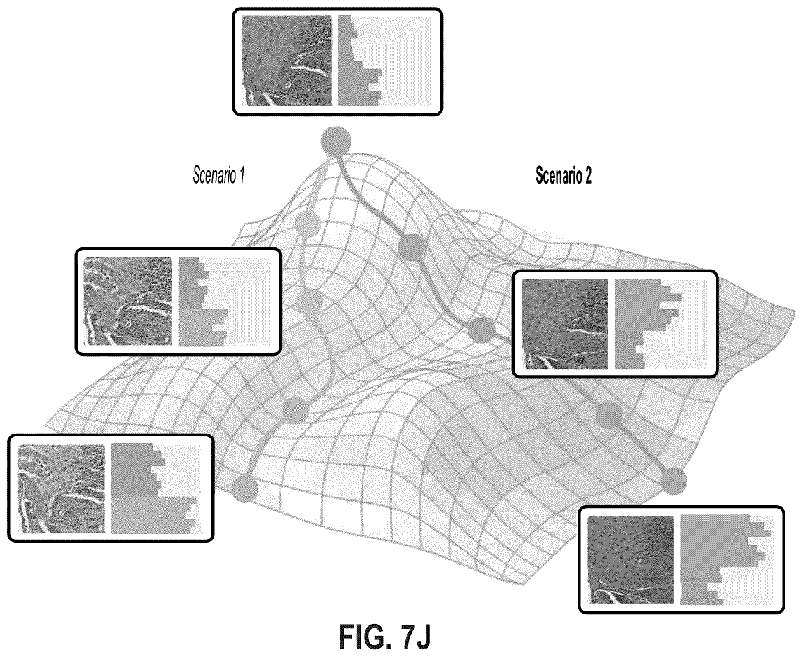

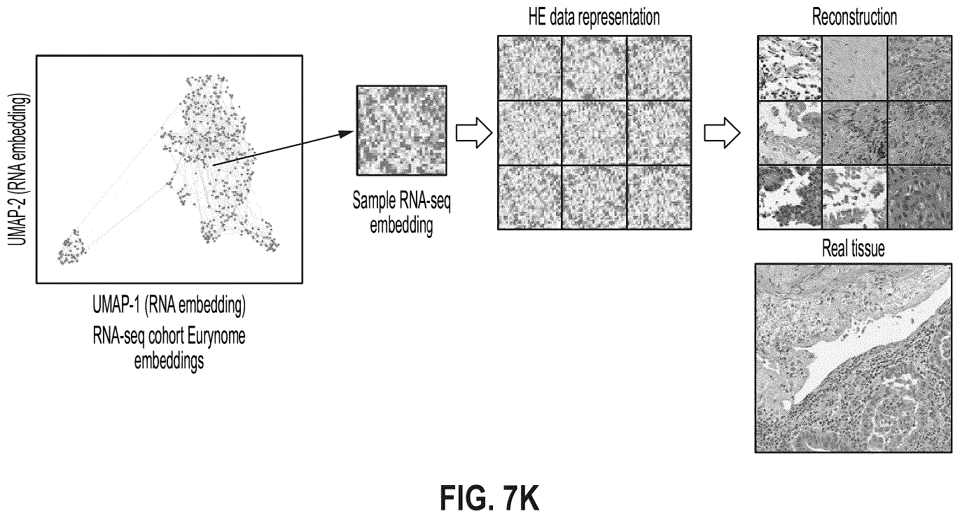

In some embodiments, the generated data 128 is used to simulate the effects of a biological alteration or therapeutic intervention on the subject 102. For example, the input data 108 provided to the trained multimodal generative model 124 may be perturbed and the trained multimodal generative model 124 may be used to generate data of the target modality corresponding to the perturbed input. For example, RNA expression data may be perturbed by modulating the expression levels of particular genes in the RNA expression data. By comparing the generated data 128 corresponding to the perturbed and unperturbed inputs, the effects of the biological alteration on the subject's biological profile may be assessed in silico. This perturbation-based approach may be used for virtual hypothesis testing regarding the predicted effects of therapeutic agents, discovery of imaging biomarkers associated with specific molecular states, and exploration of genotype-phenotype relationships, as described herein with respect to FIGS. 5A, 5C, 5D, and 7F-7J.

In some embodiments, the generated data 128 is used to model the subject's trajectory through a high-dimensional biological state landscape. For example, the input data 108 may be iteratively modified and the trained multimodal generative model 124 may be used to generate corresponding data of the target modality at each step, producing a sequence of generated outputs that traces a path through the biological state space. This trajectory modeling approach may be used to simulate disease progression, model the predicted response to a therapeutic intervention over time, or identify alternative biological trajectories that the subject may follow under different conditions, as described herein with respect to FIGS. 5B and 7J.

In some embodiments, the generated data 128 and (optionally) the input data 108 is used to characterize drug-associated biology for a therapeutic agent. For example, the generated data 128 may be used to identify response drivers, resistance programs, and mechanisms of action associated with a particular therapeutic agent. In some embodiments, the generated data 128 may be used to position the subject 102 within a broader biological landscape that includes subjects treated with standard-of-care and emerging therapies, enabling an assessment of where the therapeutic agent's biology is differentiated, how prior therapies and resistance states reshape the relevant patient population, and which subgroups of subjects are most likely to derive unique benefit from the therapeutic agent. Such characterization may enable a more rigorous and transferable analysis of drug-associated biology than conventional biomarker workflows that rely only on cohort-level differential analysis or small-sample supervised models.

In some embodiments, the generated data 128 and (optionally) the input data 108 is used to support patient identification and trial enrichment strategies. For example, the generated data 128 may be used to identify a practical patient identification strategy using widely available testing modalities and clinically feasible turnaround times, in order to support trial enrichment, enrollment efficiency, and patient selection. In some embodiments, a pretrained multimodal generative model may be fine-tuned to project-specific endpoints, such as treatment response, resistance, toxicity, or mechanism-of-action questions, enabling the model to specialize its learned biological representations for a specific therapeutic context while reducing overfitting relative to models trained from scratch on limited trial datasets.

In some embodiments, the generated data 128 is used as input to one or more additional machine learning models. For example, the generated data 128 may be used as input to a downstream classification, clustering, or regression model, or as training data for a downstream model. The model may be trained for any suitable task informed by the generated data, as aspects of the technology described herein are not limited in this respect.

In some embodiments, the generated data 128 is used to generate a clinical report for the subject 102. For example, the report may summarize the subject's biological and clinical profile, which may encompass input data 108 and generated data 128. Additionally or alternatively, the report may characterize the subject's tumor microenvironment, predicted treatment response, and/or disease trajectory. Such reports may be provided to a clinician to inform treatment decisions, for example.

It should be appreciated that the downstream tasks described above are merely examples, and that the generated data 128 may be used in any suitable downstream task, as aspects of the technology described herein are not limited to any particular downstream application.

Generating Tissue Image Data from RNA Expression Data

FIG. 1B is a diagram of an illustrative technique 130 for generating a tissue image from RNA expression data for a subject, according to some embodiments of the technology described herein. Illustrative technique 130 shown in FIG. 1B is an example of illustrative technique 100 shown in FIG. 1A, in which the target modality is tissue image data and the input data includes RNA expression data. As shown in FIG. 1B, illustrative technique 130 may include processing RNA expression data 134 for a subject 102 using computing device(s) 110 to generate a tissue image 148 for the subject 102. The processing may include: (a) encoding the RNA expression data, at act 136, to obtain the representation 138 of the RNA expression data, (b) processing the representation 138 using a trained adapter to obtain an adapted representation 142 of the RNA expression data, and (c) generating the tissue image 148 by processing, using the trained multimodal generative model 144, the noise input 146 and the adapted representation 142.

The RNA expression data 134 may be obtained or may have been previously obtained by processing one or more biological samples 104 from the subject 102 using at least one sequencing platform 132. The biological sample(s) 104 may include any suitable biological sample from which RNA can be extracted such as, for example, tissue samples, blood-derived samples, other fluid samples (e.g., saliva, urine, etc.), and/or single-cell samples, among others. The type of biological sample used may depend on the clinical context and the availability of sample material.

RNA expression data 134 may be derived from any suitable platform or technology capable of quantifying RNA expression from the biological sample 104. For example, the RNA expression data may be derived from sequencing platform 132. Examples of sequencing platforms include a next-generation sequencing (NGS) platform, a single-cell RNA sequencing (scRNA-seq) platform, a spatial transcriptomics platform, a microarray-based expression profiling platform, a cell-free RNA (cfRNA) profiling platform, a targeted gene expression profiling platform, and a long-read sequencing plaform. In some embodiments, the sequencing platform 132 may output raw sequencing reads that are subsequently aligned to a reference genome and quantified at the gene or transcript level to produce the RNA expression data 134. In some embodiments, the RNA expression data 134 has been previously obtained for the subject 102 (e.g., as part of routine clinical care or a prior research study) and is obtained from a data store for processing by computing device(s) 110.

The RNA expression data 134 may include data that reflects the transcriptional state of a biological sample 104. For example, the RNA expression data 134 may reflect the levels at which genes are being expressed in the cells of the sample 104 at the time the sample was obtained. The RNA expression data 134 may comprise expression values for a plurality of genes. For example, the RNA expression data 134 may include expression values for at least 1,000 genes, at least 2,500 genes, at least 5,000 genes, at least 10,000 genes, at least 15,000 genes, at least 20,000 genes, or any suitable number of genes between 1,000 and 20,062. In some embodiments, the RNA expression data 134 may be represented as a vector of numeric expression values, where each value corresponds to the measured or estimated abundance of a particular gene or transcript in the sample.

In some embodiments, the RNA expression data 134 may be preprocessed prior to encoding, at act 136. Preprocessing may include one or more of the following operations, depending on the sequencing platform used and the requirements of the trained encoder. Raw expression values (e.g., sequencing counts) may be normalized to account for differences in sequencing depth and gene length. For example, raw expression values may be normalized by computing transcript-per-million (TPM), fragments per kilobase million (FPKM), or counts per million (CPM) values. Additionally or alternatively, the raw or normalized expression values may be transformed by applying a log transformation (e.g., to reduce the dynamic range of the data). In some embodiments, genes with low median expression or non-coding transcripts may be filtered out, reducing the dimensionality of the expression vector. It should be appreciated that the specific preprocessing steps applied may vary depending on the modality, the sequencing platform, and the requirements of the downstream encoding and generation steps, as aspects of the technology described herein are not limited to any particular preprocessing approach. Examples of RNA expression data, and techniques for obtaining and pre-processing RNA expression data, are described herein, including at least in the section entitled “RNA Expression Data.”

As shown in FIG. 1B, processing the RNA expression data 134 includes encoding the RNA expression data 134, at act 136, using a trained encoder to obtain a representation 138 of the RNA expression data 134. The trained encoder used to encode the RNA expression data 134 may be any suitable encoder capable of encoding RNA expression data into a numeric representation. In some embodiments, the trained encoder is the encoder component of a trained encoder-decoder pair that has been trained to reconstruct RNA expression data from a compact latent representation, as described herein with respect to FIG. 1A. For example, the trained encoder may be the encoder portion of an autoencoder. Examples of autoencoders include as a latent image autoencoder (LIAE), a variational autoencoder (VAE), and a vector-quantized VAE (VQ-VAE). As described herein, an LIAE is a type of convolutional autoencoder that encodes a one-dimensional RNA expression vector into a two-dimensional latent representation. For example, the LIAE may project the expression vector into a lower-dimensional space using a linear layer, reshape the projected representation into a two-dimensional spatial feature map, and then process the feature map through a series of convolutional residual blocks to produce the final latent representation. The two-dimensional latent representation may be well-suited for use with generative models that operate on two-dimensional spatial feature maps, such as the latent diffusion models described herein. Additional examples of encoders trained to encode RNA expression data are described herein including at least with respect to FIGS. 6G-6I.

The representation 138 of the RNA expression data may take any suitable form that captures the biologically relevant features of the RNA expression data 134. For example, the representation 138 may take the form of a one-dimensional vector or two-dimensional feature map. The form may depend on the encoder architecture. For example, an LIAE may produce a two-dimensional feature map while a VAE or VQ-VAE may produce a one-dimensional vector.

As shown in FIG. 1B, processing the RNA expression data 134 further includes processing the representation 138, at act 140, using a trained adapter to obtain an adapted representation 142 of the RNA expression data. The trained adapter transforms the representation 138 into a form that is compatible with and suitable for conditioning the trained multimodal generative model 144. For example, the trained adapter may comprise one or more linear projection layers, non-linear activation layers, normalization layers, and/or reshaping operations configured to map the representation 138 to a sequence of tokens of an embedding dimension compatible with the cross-attention layers of a denoising diffusion model. An example of a trained adapter used to process RNA expression representations is described herein including at least with respect to FIG. 6J. The example adapter of FIG. 6J transforms a representation of RNA expression data into a sequence of tokens suitable for use as a key-value input to the cross-attention layers of a denoising diffusion model.

As shown in FIG. 1B, the adapted representation 142 of the RNA expression data is used to condition the trained multimodal generative model 144, which generates a tissue image 148 for the subject 102 from a noise input 146. In some embodiments, the trained multimodal generative model 144 is a denoising diffusion model (e.g., a latent diffusion model) that iteratively denoises the noise input 146 conditioned on the adapted representation 142 to generate the tissue image or a representation of the tissue image. In some embodiments, the denoising diffusion model includes a neural network backbone such as, for example, a UNet or transformer. The neural network backbone may be pre-trained. For example, the pre-trained backbone may be a pre-trained UNet, such as Stable Diffusion 1.5. In some embodiments, the neural network backbone includes a plurality of cross-attention layers. The model may be conditioned on the adapted representation 142 of the RNA expression data via the cross-attention layers. For example, the cross-attention layers may incorporate the adapted representation 142 as key-value input at each denoising timestep, so that the neural network's noise prediction is guided by the RNA expression data 134. For example, as shown in FIG. 4D, the cross-attention layers of the multimodal generative model are conditioned on the conditioning vector that represents the adapted representations of the image, expression, and CRNA data. In some embodiments, when the neural network backbone is pre-trained, it may include original cross-attention layers and supplementary cross-attention layers. The model may be conditioned on the adapted representation 142 of the RNA expression data via the supplementary cross-attention layers and (optionally) the original cross-attention layers.

The output of the trained multimodal generative model 144 may include a tissue image 148 and/or a representation of the tissue image 148. In some embodiments, when the output is a representation of the tissue image, the representation is subsequently decoded (e.g., a pre-trained decoder) into a tissue image 148 by a decoder, such as a decoder of an encoder-decoder pair. For example, the decoder may be a decoder portion of an autoencoder such as a VAE (e.g., the VAE from Stable Diffusion 1.5). The tissue image 148 output by the trained multimodal generative model 144 is a synthetic tissue image, in that it was not obtained by measuring (e.g., imaging) the biological sample(s).

As used herein, the term “tissue image” refers to any image depicting biological tissue, a portion thereof, or constituent cells, regardless of imaging modality, staining, magnification, dimensionality, or scale. A tissue image may be obtained by, or derived from, brightfield microscopy, fluorescence microscopy, confocal microscopy, electron microscopy, multiphoton microscopy, or any other imaging modality suitable for visualizing biological tissue. A tissue image may depict tissue that has been stained, labeled, or otherwise processed using one or more histological, histochemical, immunohistochemical (IHC), immunofluorescence (IF), or molecular staining or labeling techniques (e.g., hematoxylin and eosin (H&E), IHC stains targeting one or more proteins of interest, multiplexed IHC or IF panels, in situ hybridization (ISH), fluorescence in situ hybridization (FISH), etc.). A tissue image may depict tissue that is unstained or label-free. A tissue image may be a two-dimensional image, a three-dimensional image (e.g., a volumetric reconstruction), or a multi-channel image (e.g., comprising multiple imaging channels corresponding to different stains, markers, or wavelengths). A tissue image may be a whole-slide image, a region of interest extracted from a whole-slide image, a single tile or patch (e.g., 256×256 pixels, 512×512 pixels, 1024×1024 pixels, or any other suitable size), a tiled or stitched composite image, or a thumbnail or downsampled representation of any of the foregoing. A tissue image may depict tissue obtained from any organ, tissue type, or anatomical location, including without limitation tumor tissue, healthy tissue, biopsy specimens, surgical resections, fine-needle aspirates, cytology preparations, and tissue microarrays. A tissue image may be a real image acquired from a physical biological sample, a synthetic or generated image produced by a computational model (including by the methods described herein), or a hybrid or augmented image combining real and synthetic content. The foregoing examples are illustrative and non-limiting, and the term “tissue image” is intended to encompass any image of biological tissue suitable for use in connection with the methods, systems, and computer-readable media described herein.

In some embodiments, multiple tissue images are generated for the subject using illustrative technique 130. The multiple tissue images may be generated from the same RNA expression data 134. For example, the multiple tissue images may be generated by processing different random noise inputs 146 through the trained multimodal generative model 144 conditioned on the adapted representation 142 of the RNA expression data 134. The multiple tissue images may provide a distribution of plausible tissue morphologies consistent with the subject's transcriptomic profile.

The generated tissue image 148 may be used to perform one or more downstream tasks, as described herein with respect to act 129 of illustrative technique 100. For example, the generated tissue image 148 may be used to characterize the subject's tumor microenvironment, identify morphological features associated with specific molecular states, predict response to a therapeutic agent, identify the subject as a member of a clinical trial cohort, and/or support in silico simulation of the morphological consequences of biological perturbations to the RNA expression data. Examples of tissue images generated according to illustrative technique 130 are shown in FIGS. 7A-7C, and examples of tissue images generated from perturbed RNA expression data are shown in FIGS. 7F-7J. Additional details of examples of components used to implement illustrative technique 130 are described herein with respect to FIGS. 6A-6J.

Generating Representations of RNA Expression Data from Tissue Image Data

FIG. 1C is a diagram of an illustrative technique 150 for generating, from a tissue image 158, a representation of RNA expression data 176 and an output 178 based on the representation 176 of the RNA expression data for a subject, according to some embodiments of the technology described herein. Illustrative technique 150 is an example of illustrative technique 100 shown in FIG. 1A, in which the target modality is RNA expression data and the input data includes tissue image data. As shown in FIG. 1C, illustrative technique 150 may include processing a tissue image 158 of a subject 102 using computing device(s) 110 to generate a representation 176 of RNA expression data and an output 178 based on the representation 176. The processing may include: (a) generating image portions 162-1 through 162-N from the tissue image 158, at act 160; (b) encoding the image portions, at act 164, to obtain representations 166-1 through 166-N of the image portions; (c) processing the representations 166-1-166-N using a trained adapter, at act 168, to obtain an adapted representation 170 of the tissue image 158; (d) generating the representation 176 of the RNA expression data by processing, using the trained multimodal generative model 172, a noise input 174 and the adapted representation 170; and (e) generating the output 178 from the representation 176 of the RNA expression data.

The tissue image 158 may be obtained or may have been previously obtained by processing one or more biological samples 104 from the subject 102 using at least one imaging platform 156. The biological sample(s) 104 may include any suitable biological sample from which a tissue image can be obtained. For example, the biological sample(s) may include a tissue biopsy, a surgical resection, or any other tissue specimen that has been prepared for imaging. Example techniques for preparing a tissue specimen for imaging include sectioning, staining, and/or mounting the tissue specimen on a glass slide. It should be appreciated that aspects of the technology described herein are not limited to any particular type of biological sample or tissue preparation method.

The imaging platform 156 may be any suitable platform capable of producing a digital image of the tissue sample. For example, the imaging platform 156 may be a whole-slide image scanner that digitizes a glass histopathology slide at a suitable magnification (e.g., 5×, 10×, 20×, etc.) using brightfield or fluorescence imaging; a fluorescence microscope or scanner used to image fluorescently labeled tissue sections, for example for immunofluorescence or multiplexed imaging; a multiplexed imaging system (e.g., a cyclic immunofluorescence (CyCIF) system, a CODEX system, etc.) that captures multiple protein markers simultaneously; or a spatial transcriptomics imaging system (e.g., 10× Genomics Visium or Xenium) that captures both a tissue image and spatially resolved expression data. In some embodiments, the tissue image 158 has been previously obtained for the subject 102 (e.g., as part of routine clinical care or a prior pathological examination) and is retrieved from a data store or medical imaging archive for processing by computing device(s) 110. It should be appreciated that aspects of the technology described herein are not limited to any particular imaging platform or imaging modality.

The tissue image 158 may include any suitable type of tissue image. For example, the tissue image 158 may be a brightfield image or a fluorescence image. Examples of brightfield images include a hematoxylin and eosin (H&E) stained histopathological image and an immunohistochemistry (IHC) image. Examples of fluorescence images include an immunofluorescence image or a multiplexed imaging image (e.g., an image produced by a cyclic immunofluorescence (CyCIF) system or a CODEX system). In some embodiments, the tissue image 158 is a whole-slide image (WSI). The tissue image 158 may be of any suitable size and magnification.

In some embodiments, the tissue image 158 may be preprocessed prior to generating image portions at act 160. For example, the tissue image 158 may be preprocessed by applying stain normalization to reduce variation in staining intensity across slides, and/or by applying tissue detection to identify and exclude background and artifact regions of the slide from subsequent processing.

As shown in FIG. 1C, processing the tissue image 158 includes generating a plurality of image portions 162-1-162-N from the tissue image 158, at act 160. The image portions 162-1 through 162-N may be generated by dividing the tissue image 158 into a plurality of smaller tiles that together cover the tissue region of the image. In some embodiments, the tiles are non-overlapping. The image portions may be of any suitable size that is smaller than the tissue image. For example, an image portion may be a size between 128×128 pixels and 2,048×2,048 pixels. The number of image portions N may vary depending on the size of the tissue image and the tile size used, and may range from tens to thousands of image portions.

In some embodiments, the image portions may be pre-processed before being encoded at act 164. The pre-processing techniques may include filtering out one or more image portions (i.e., excluding them from further analysis. For example, image portions that depict less than a threshold amount of tissue may be filtered out. Such image portions may depict artifact, background, and/or non-tissue regions. The threshold amount may be measured based on the percentage of the image portion (e.g., the percentage of pixels of the image portion) that depict tissue. For example, the threshold amount may be between 25% and 100%. The pre-processing may be performed using a tissue detection algorithm (e.g., image segmentation) that identifies tiles containing sufficient tissue content, or using any other suitable techniques, as aspects of the technology described herein are not limited in this respect.

As shown in FIG. 1C, processing the tissue image 158 further includes encoding the image portions 162-1-162-N, at act 164, using a trained encoder to obtain representations 166-1-166-N of the image portions. For example, a trained encoder may be used to encode each of at least some (e.g., all image portions, image portions not filtered out during pre-processing, etc.) to obtain a respective representation for each image portion. For example, this may include (i) encoding image portion 162-1 using the trained encoder to obtain representation 166-1 of the image portion, (ii) encoding image portion 162-2 using the trained encoder to obtain representation 166-2 of the image portion, and so on. The result is a variable length set of representations 166-1-166-N with length N, where N depends on the number of image portions 161-1-161-N encoded at act 164.

The trained encoder used to encode the image portions may be any suitable encoder capable of encoding image data into a numeric representation, as aspects of the technology described herein are not limited to any particular encoder architecture. The type of encoder used may depend on the nature of the tissue image, the resolution and magnification at which it was acquired, and the desired properties of the resulting representation.

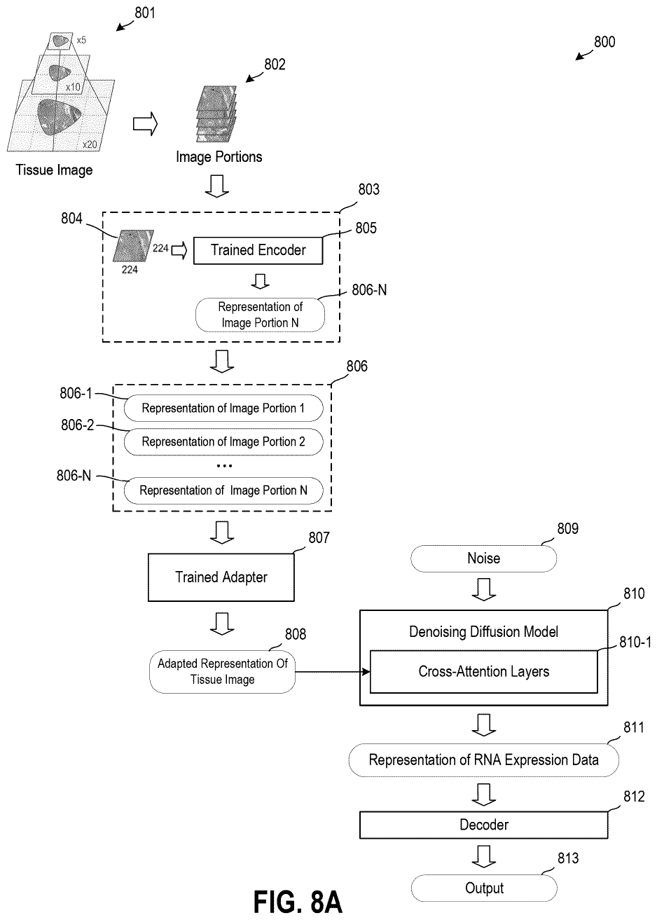

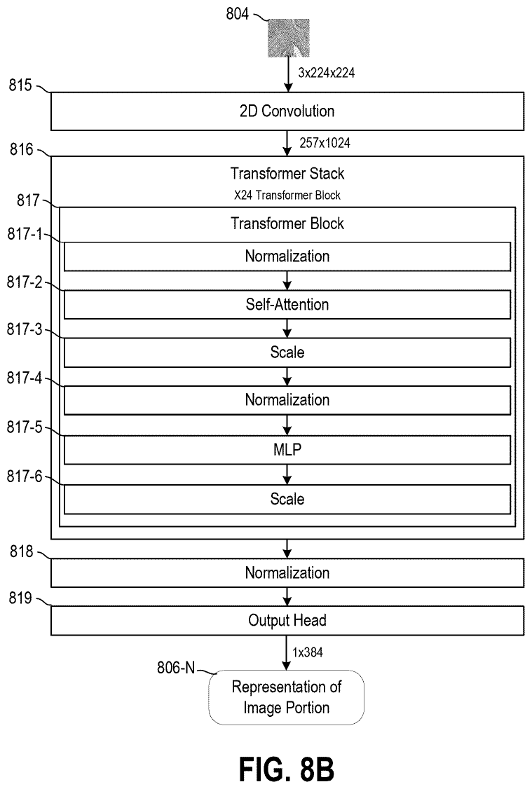

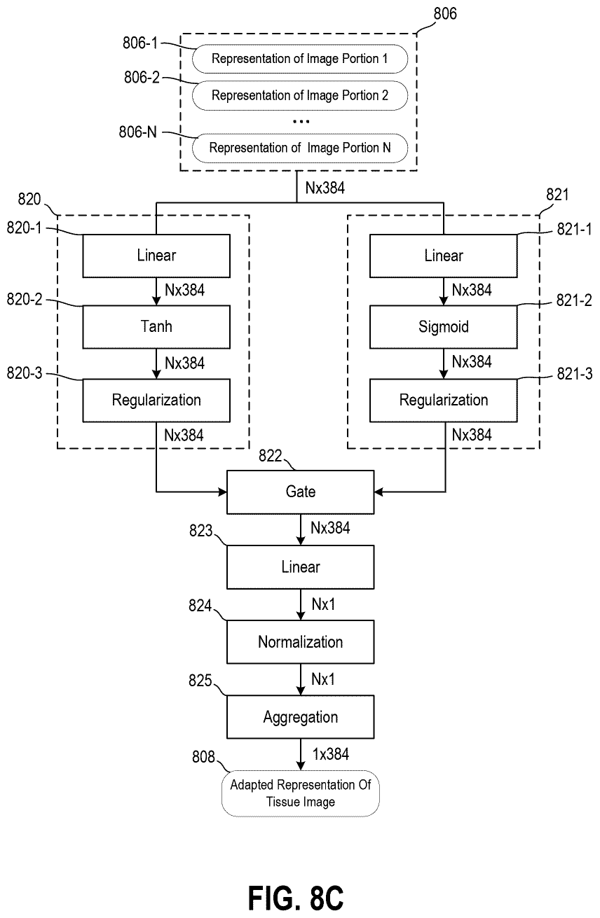

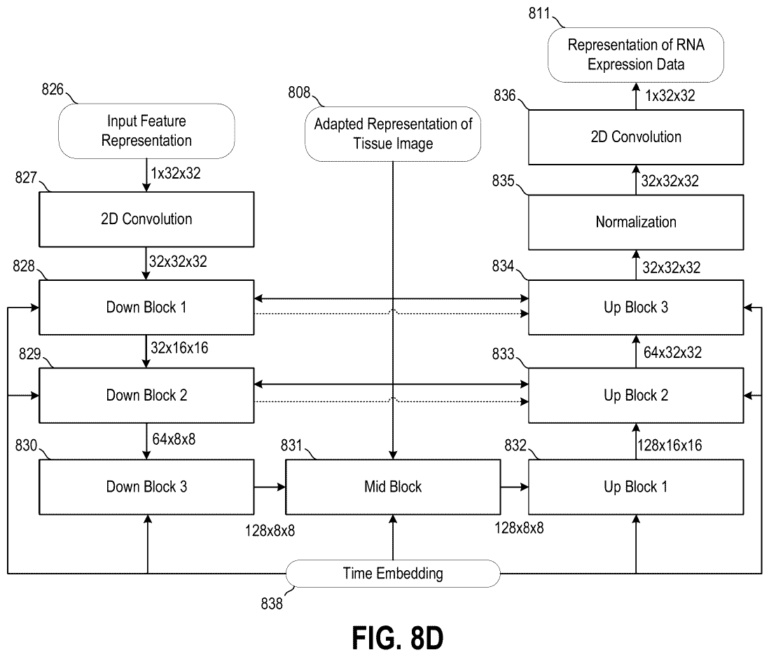

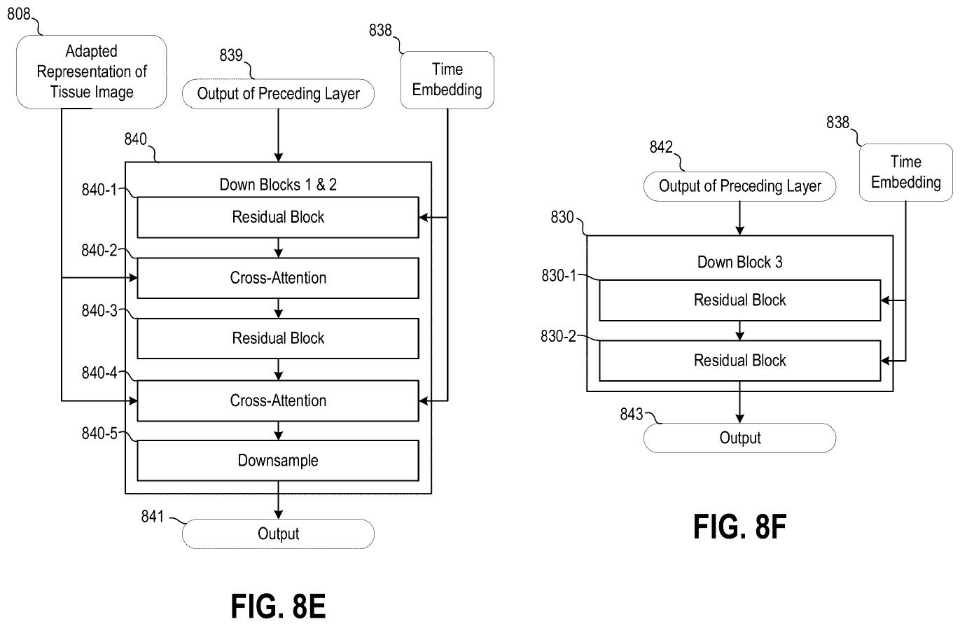

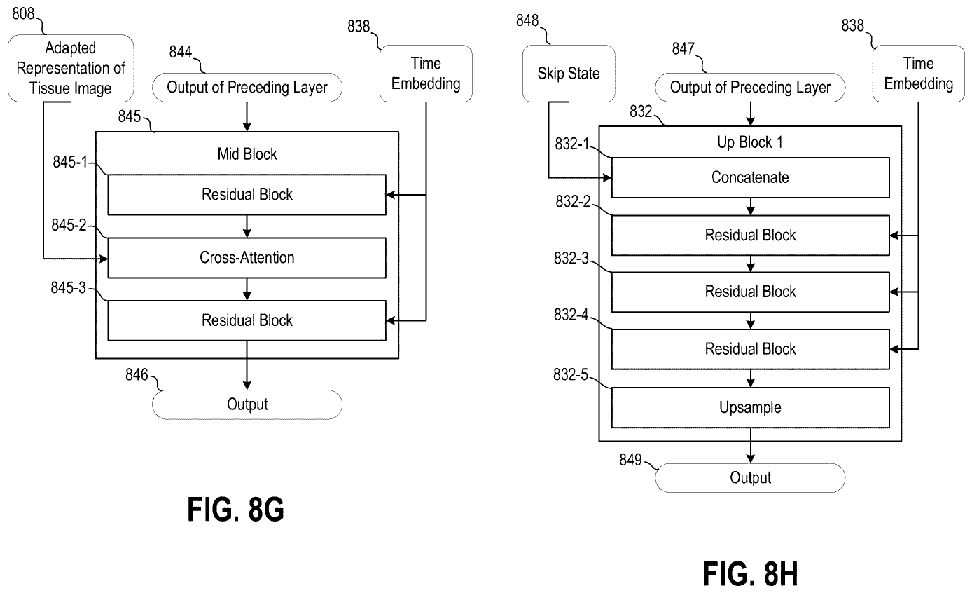

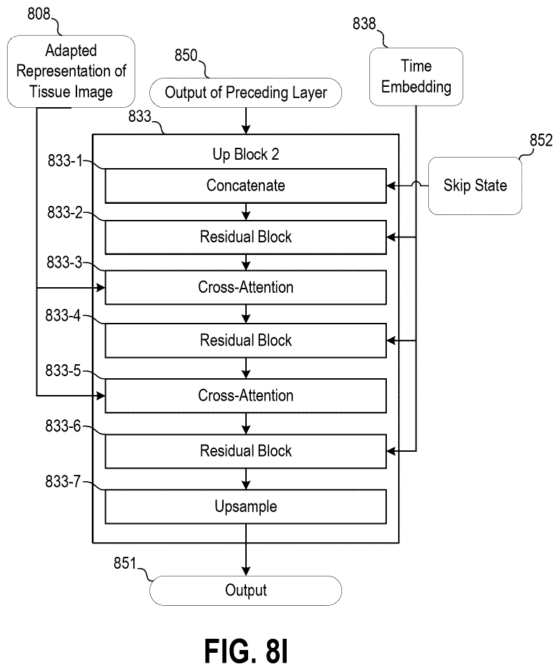

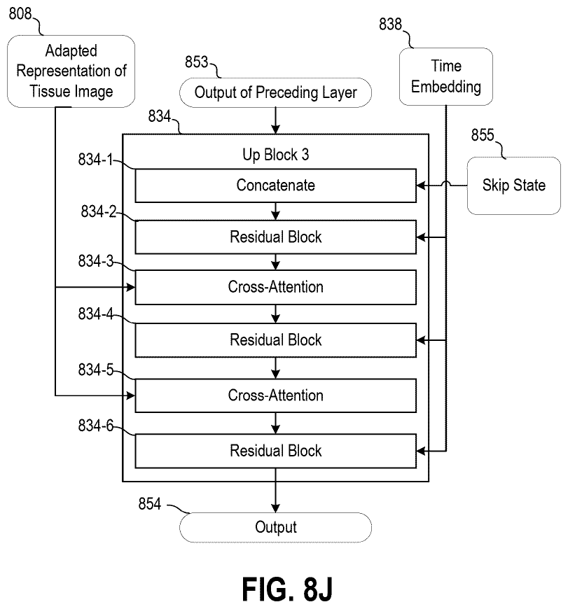

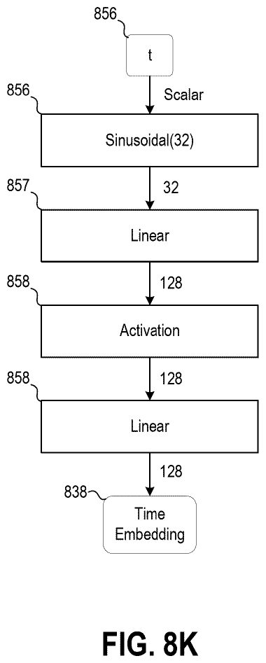

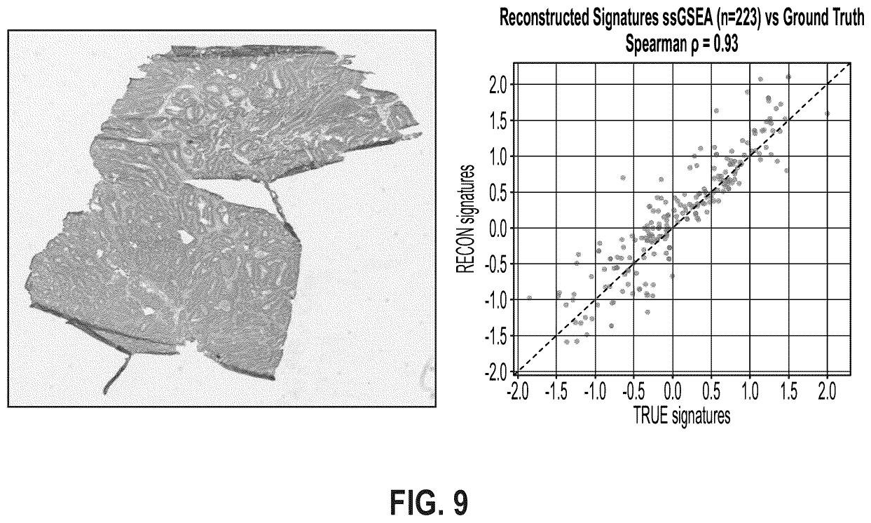

In some embodiments, the trained encoder is a vision transformer (ViT). The ViT may be suitable for encoding tissue image portions because they can capture, through their self-attention mechanism, both local morphological features, such as cellular composition and nuclear morphology, as well as long-range spatial relationships within the image portion. The ViT may be in any suitable configuration such as, for example, ViT-small (VIT-S), ViT-base (ViT-B), ViT-large (ViT-L), ViT-Huge (ViT-H), and ViT-giant (ViT-g). The VIT models are described by Dosovitskiy, A., et al. The trained encoder may vary in scale, training data, and provenance, ranging from smaller ViTs trained on general image corpora to large ViTs pre-trained on domain-specific tissue image corpora, which may be referred to herein as pre-trained foundation models. In some embodiments, the vision transformer is trained using a self-supervised learning method in which the encoder learns to produce representations that capture meaningful morphological features of tissue without requiring manual annotations. For example, the self-supervised learning method may be trained using a teacher-student framework such as self-distillation with no labels (DINO), DINOv2, or DINOv3. An example of a vision transformer encoder used to encode tissue image portions is described herein with respect to FIGS. 8B and 9C.

In some embodiments, the trained encoder is a pre-trained foundation model. As described herein, a pre-trained foundation model may refer to a large vision transformer that has been pre-trained on a large and diverse corpus of tissue images using self-supervised or other objectives, and that produces rich, high-dimensional embeddings of tissue image portions encoding information about tissue architecture, cellular composition, morphological patterns, and other biologically relevant features. Because pre-trained foundation models may be exposed to a wide variety of tissue types, cancer types, staining protocols, and imaging conditions during pre-training, they produce generalizable representations that transfer well across different downstream tasks and datasets. Pre-trained foundation models are available in a range of ViT configurations (e.g., ViT-L, ViT-H, and ViT-g) with larger configurations generally producing richer representations at greater computational cost. Examples of pre-trained foundation models that may be used as encoders include H-Optimus-0, H-Optimus-1, Hibou, UNIv2, and Virchow, among others. H-Optimus-0 is made available by Saillard, C., et al. (“H-optimus-0” (2024). Available at GitHub.) H-Optimus-1 is made available by Bioptimus. (“H-optimus-1” (2025). Available on Hugging Face.). Hibou is described by Nechaev, D., Pchelnikov, A., & Ivanova, E. (“Hibou: A Family of Foundational Vision Transformers for Pathology.” arXiv: 2406.05074 (2024).); UNI is described by Chen, R. J., et al. (“Towards a general-purpose foundation model for computational pathology.” Nature Medicine, 30, 850-862 (2024). DOI: 10.1038/s41591-024-02857-3); and Virchow is described by Vorontsov, E., et al. “A foundation model for clinical-grade computational pathology and rare cancers detection.” Nature Medicine, 30 (10), 2924-2935 (2024). DOI: 10.1038/s41591-024-03141-0), each of which is incorporated by reference herein in its entirety. In some embodiments, the pre-trained foundation model is used as a fixed feature extractor with its weights held frozen during training of the trained adapter and/or trained multimodal generative model 172. In other embodiments, the pre-trained foundation model may be fine-tuned on domain-specific data prior to use as an encoder.

The representation of each image portion produced by the trained encoder may take any suitable form depending on the encoder architecture used. In some embodiments, a representation of an image portion may be a vector of fixed dimensionality, such as a classification (CLS) token embedding. In some embodiments, a representation of an image portion may be a sequence of patch token embeddings (e.g., one token embedding per image patch) that preserves the spatial structure of the image portion by associating each token with a specific spatial position within the image.

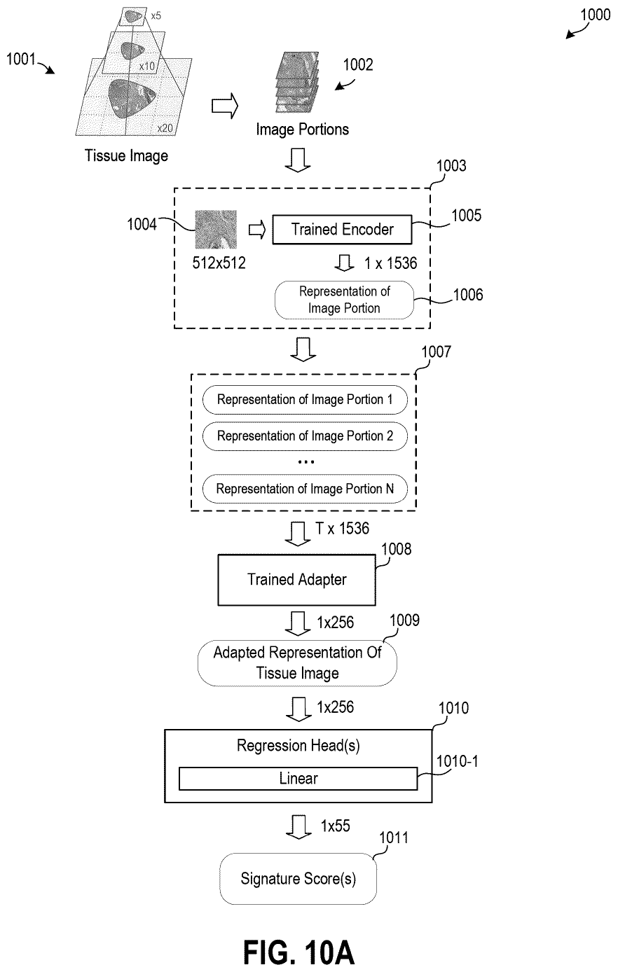

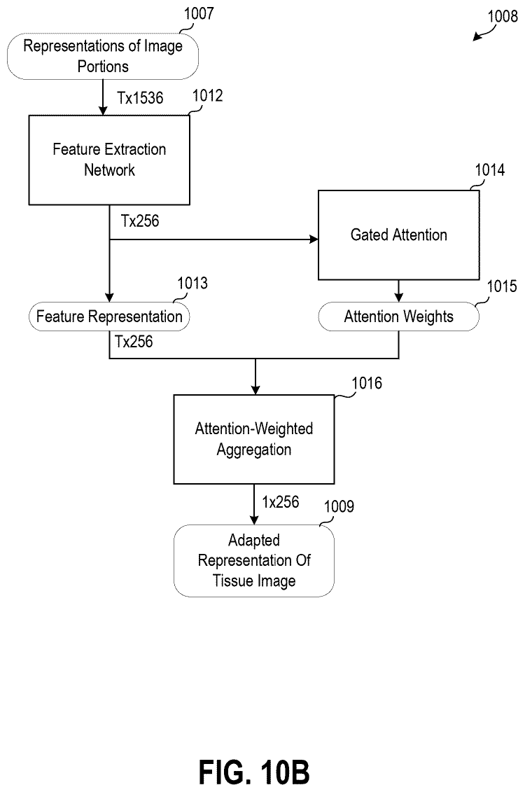

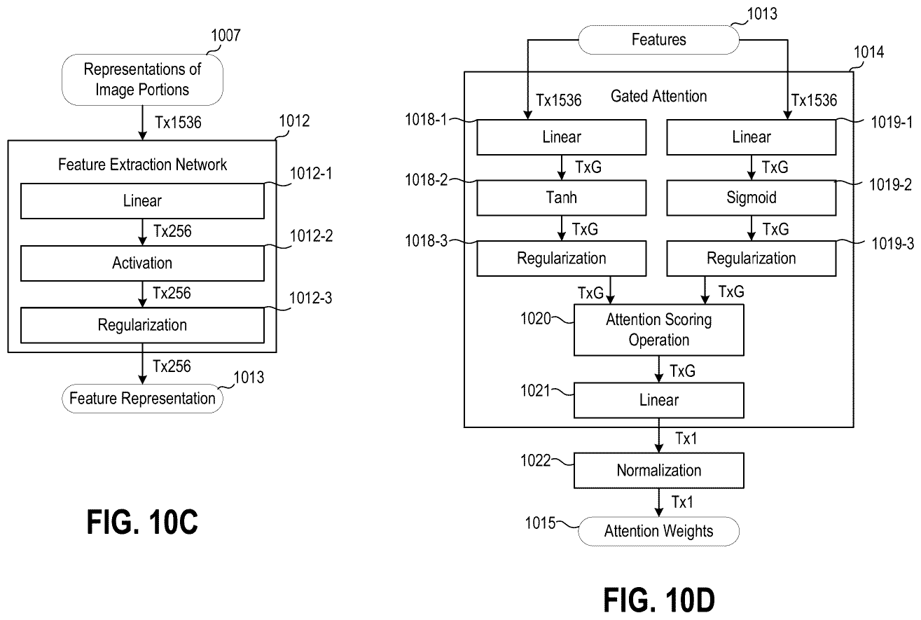

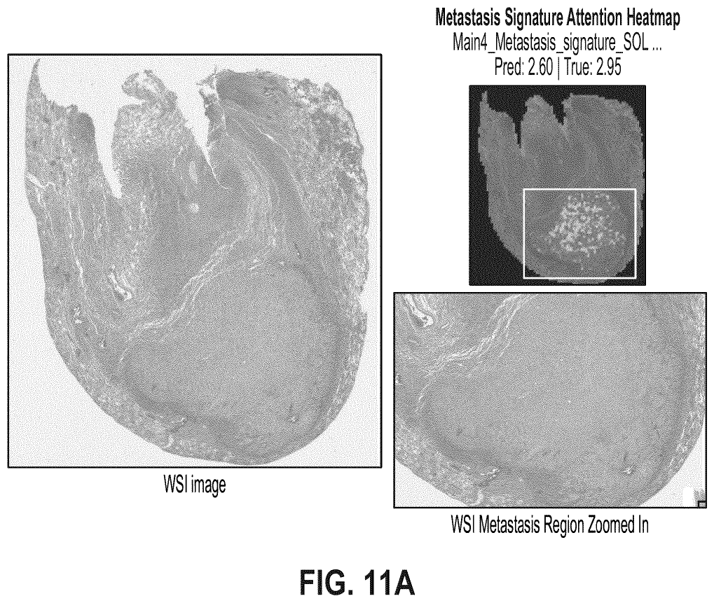

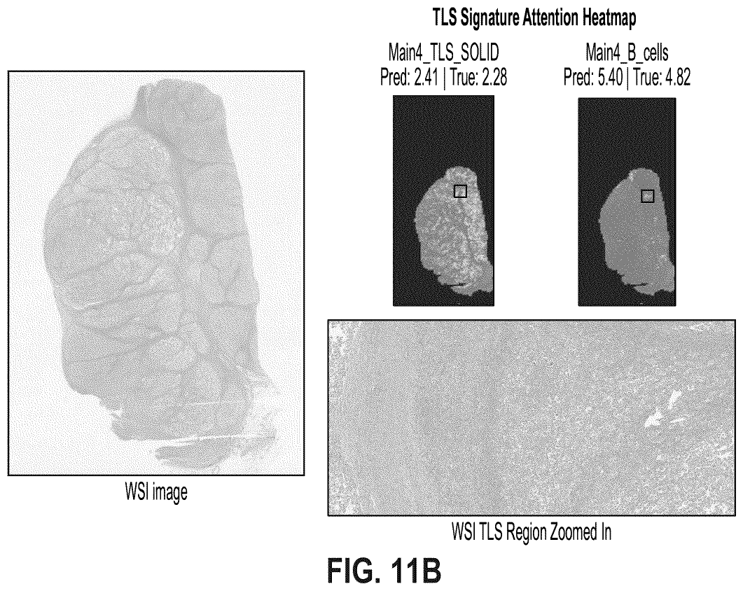

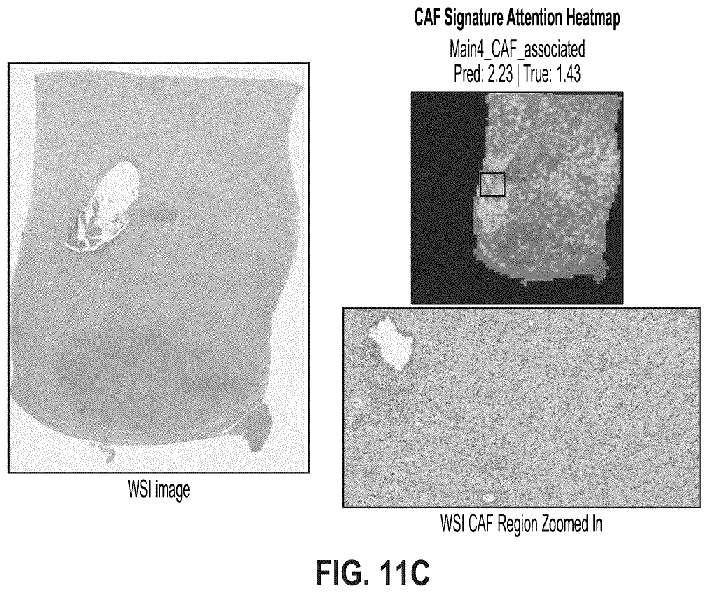

As shown in FIG. 1C, processing the tissue image 158 further includes processing the representations 166-1-166-N, at act 168, using a trained adapter to obtain an adapted representation 170 of the tissue image 158. Because the tissue image 158 is divided into a variable-length set of image portions 162-1-162-N, the trained adapter is configured to aggregate the representations 166-1-166-N of the image portions into a single adapted representation 170 of the tissue image as a whole. In some embodiments, at act 168, the trained adapter performs multiple instance learning to determine a weighted combination of the representations 166-1-166-N of the image portions to obtain an adapted representation 170 of the tissue image 158. For example, the trained adapter may use a gated attention mechanism that assigns a learned importance weight to each image portion and computes a weighted sum of the representations 166-1-166-N. In some embodiments, as a byproduct of aggregating the representations 166-1-166-N, the gated attention mechanism produces per-tile attention weights that may be used to generate spatial heatmaps identifying the regions of the tissue image most informative for the generated output, as described herein with respect to FIGS. 11A-11C. Additional examples of trained adapters used to process tissue image representations are described herein including at least with respect to FIGS. 8C and 10B.

As shown in FIG. 1C, the adapted representation 170 of the tissue image may be used to condition the trained multimodal generative model 172, which generates a representation 176 of RNA expression data for the subject 102 from a noise input 174. In some embodiments, the trained multimodal generative model 172 is a denoising diffusion model, such as a latent diffusion model, that iteratively denoises the noise input 174 conditioned on the adapted representation 170 to generate the representation 176 of the RNA expression data. In some embodiments, the neural network backbone (e.g., UNet or transformer backbone) of the denoising diffusion model may comprise a plurality of cross-attention layers. The model may be conditioned on the adapted representation 170 of the tissue image via one or more of the cross-attention layers, so that the neural network's noise prediction at each timestep is guided by the tissue image data of the subject. In some embodiments, the noise input 174 is a random tensor (e.g., drawn from a Gaussian distribution) that serves as a starting point for the reverse diffusion process. Additional details of an example trained multimodal generative model used to generate the representation of RNA expression data are described herein including at least with respect to FIGS. 8A-8K.

In some embodiments, the output of the trained multimodal generative model 172 includes a representation 176 of RNA expression data. For example, the representation 176 may be a latent representation in the latent space of a trained encoder-decoder pair. In some embodiments, multiple representations of RNA expression data are generated for the subject. For example, multiple representations of RNA expression data may be generated by processing different random noise inputs 174 through the trained multimodal generative model 172 conditioned on the same adapted representation 170. In some embodiments, the multiple representations are combined (e.g., averaged) to produce a single representation of the RNA expression data. This may reduce the variance of the generated representations and may produce a more stable estimate of the subject's transcriptomic profile. In some embodiments, the multiple representations are used individually as a set of additional or alternative samples.

As shown in FIG. 1C, an output 178 may be generated from the representation 176 of the RNA expression data. The output 178 may be any suitable output that can be derived from the representation 176 of the RNA expression data. In some embodiments, the output 178 includes decoded RNA expression data. For example, the representation 176 of the RNA expression data may be decoded, using a trained decoder, into RNA expression data in the original data space. For example, the trained decoder may be a decoder portion of a trained encoder-decoder pair (e.g., a decoder portion of a trained LIAE, VAE, or VQ-VAE). In some embodiments, the decoded RNA expression data includes expression level values for a plurality of genes. For example, the plurality of genes may include any suitable number of genes between 5,000 and 20,000. In some embodiments, the output 178 includes normalized and/or log-transformed gene expression level values. In some embodiments, the output 178 includes one or more values that may be derived from RNA expression data. For example, the one or more values may include one or more signature scores for one or more gene groups. The signature scores may be derived by applying a gene set scoring method, such as single-sample gene set enrichment analysis (ssGSEA) or any other suitable scoring method, to RNA expression data derived from the representation 176. In some embodiments, the output 178 comprises a classification, prediction, or score for the subject derived from the representation 176 or the decoded RNA expression data. It should be appreciated that the output 178 is not limited to any particular type of output, as aspects of the technology described herein are not limited in this respect.

The generated representation 176 and/or output 178 may be used to perform one or more downstream tasks, as described herein with respect to act 129 of illustrative technique 100. For example, the representation 176 and/or output 178 may be used to characterize the subject's tumor microenvironment, predict response to a therapeutic agent, identify the subject as a member of a clinical trial cohort, stratify the subject into a predicted responder or non-responder group, or monitor disease progression over time-all from a tissue image and corresponding synthetic RNA expression data, without requiring a molecular assay. Additional details of the components used to implement illustrative technique 150 are described herein with respect to FIGS. 8A-9.

### Generating Output Values for a Subject From Tissue Image Data

FIG. 1D is a diagram of an illustrative technique 180 for generating, from a tissue image 158, output values 190 for a subject 102, according to some embodiments of the technology described herein. Illustrative technique 180 is an example of illustrative technique 100 shown in FIG. 1A, in which the trained multimodal generative model directly generates a plurality of output values 190 from an adapted representation 184 of a tissue image 158. As shown in FIG. 1D, illustrative technique 180 may include processing a tissue image 158 of a subject 102 using computing device(s) 110 to generate one or more output values 190 for the subject 102. The processing may include: (a) generating image portions 162-1-162-N from the tissue image 158, at act 160; (b) encoding the image portions, at act 164, to obtain representations 166-1-166-N of the image portions; (c) processing the representations 166-1-166-N using a trained adapter, at act 182, to obtain an adapted representation 184 of the tissue image 158; and (d) generating the output value(s) 190 by processing, using the trained multimodal generative model 186, the adapted representation 184.

Illustrative technique 180 differs from illustrative technique 150 shown in FIG. 1C in that the trained multimodal generative model 186 directly generates the output value(s) 190 from the adapted representation 184 of the tissue image, without first generating a representation of RNA expression data from a noise input. Additionally, as described herein with respect to FIG. 1A, in some embodiments, the trained multimodal generative model may be a regression model that directly maps an adapted representation of the input data to the one or more output values, rather than a diffusion model that iteratively denoises a noise input to generate data of the target modality. Illustrative technique 180 is an example of such an embodiment.

Multiple aspects of illustrative technique 180 are described herein with respect to illustrative technique 150 shown in FIG. 1C, and the same description applies here. For example, the following aspects of illustrative technique 180 are described with respect to FIG. 1C: the tissue image 158; the biological sample(s) 104 from which the tissue image 158 may be obtained; the imaging platform 156 used to obtain the tissue image 158; techniques for generating image portions 162-1-162-N; techniques for pre-processing the tissue image 158; techniques for pre-processing the image portions 162-1-162-N; techniques for encoding the image portions 162-1-162-N, at act 164, using a trained encoder to obtain representations 166-1-166-N of the image portions; the types of encoders that may be used; and the forms that the representations 166-1-166-N may take.

As shown in FIG. 1D, processing the tissue image 158 further includes processing the representations 166-1-166-N, at act 182, using a trained adapter to obtain an adapted representation 184 of the tissue image 158. The trained adapter may be configured to aggregate the variable-length set of representations 166-1-166-N of the image portions 162-1-162-N into a single adapted representation 184 of the tissue image 158. In some embodiments, the trained adapter comprises a feature extraction network and at least one gated attention network, as described herein with respect to FIGS. 10B, 10C, and 10D.

In some embodiments, the feature extraction network processes each of at least some (e.g., all) of the representations 166-1-166-N to produce a corresponding feature representation for each image portion. For example, the feature extraction network may comprise one or more linear projection layers and one or more non-linear activation layers (e.g., a linear projection layer followed by a ReLU activation and a dropout layer) that project each tile-level representation from the dimensionality of the encoder output to a lower hidden dimensionality. For example, the feature extraction network may project each tile-level representation from a dimensionality (e.g., 1,536) produced by a pre-trained foundation model (e.g., H-Optimus-0) to a lower hidden dimensionality H (e.g., 256). The feature extraction network may be applied independently and identically to all N tile-level representations, producing a set of N feature representations of dimensionality H that serve as inputs to the gated attention network.

In some embodiments, the at least one gated attention network determines a respective attention weight for each of at least some (e.g., all) of the image portions. The trained adapter may use those attention weights to compute a weighted combination of the feature representations produced by the feature extraction network to obtain the adapted representation 184. In some embodiments, the gated attention network is a trained component with parameters that are updated during training. The gated attention network may implement a gated attention mechanism comprising two parallel branches that each independently processes the feature representations produced by the feature extraction network. For example, a first branch may apply a Tanh activation to a linear projection of each feature representation, providing a sign-sensitive activation that captures the direction of each feature's contribution. A second branch may apply a Sigmoid activation to a separate linear projection of each feature representation, acting as a gate that selectively suppresses or amplifies the output of the first branch. In some embodiments, the outputs of the two branches may be combined (e.g., by element-wise multiplication) to produce a gated feature representation for each of at least some (e.g., all) of the image portions. The gated feature representation may be passed through a linear attention scoring layer to produce a scalar attention logit for each image portion. In some embodiments, the scalar attention logits are normalized across all N image portions (e.g., using a softmax operation), producing a probability distribution over image portions that sums to one.

In some embodiments, the trained adapter may computer the adapted representation 184 as the attention-weighted sum of the feature representations, using the (normalized) attention weights produced by the gated attention network. Thus, image portions assigned higher attention weights may contribute more strongly to the adapted representation than those assigned lower attention weights. In this way, the trained adapter learns to identify and up-weight the image portions that are most morphologically informative for the downstream prediction task, while suppressing uninformative background or artifact regions.

In some embodiments, the trained adapter comprises a single shared gated attention network that produces a single set of attention weights used to compute a single adapted representation 184, from which all output values 190 are predicted. In other embodiments (e.g., in a multi-head variant), the trained adapter comprises a respective gated attention network for each output value to be predicted. For example, when the output values include signature scores for gene groups, a respective gated attention network may be provided for each gene group for which a signature score is to be predicted. In embodiments using the multi-head variant, a (e.g., each) gated attention network may independently determine its own set of attention weights and its own adapted representation, enabling each output value to be predicted from a different weighted combination of the tile-level representations. This allows the model to identify and attend to different morphological regions of the tissue image for different output values (e.g., attending to lymphocyte-rich regions for immune cell signature scores and to stromal regions for cancer-associated fibroblast signature scores) providing per-output spatial attention and interpretability.

In some embodiments, as a byproduct of the gated attention aggregation, the gated attention network may produce per-tile attention weights (e.g., either a single set of attention weights in the shared variant, or a respective set of attention weights per output value in the multi-head variant) that may be projected back onto the original tissue image 158 to generate spatial attention heatmaps. The spatial attention heatmaps may be used to identify the morphological regions of the tissue most informative for each predicted output value, as described herein with respect to FIGS. 11A-11C. Additional examples of the trained adapter architecture are described herein including at least with respect to FIGS. 10B, 10C, and 10D.

As shown in FIG. 1D, the adapted representation 184 of the tissue image is provided as input to the trained multimodal generative model 186, which directly generates the plurality of output values 190 for the subject 102 from the adapted representation 184. In some embodiments, the trained multimodal generative model 186 is a regression model comprising one or more regression heads (e.g., one or more linear layers) that map the adapted representation 184 to the plurality of output values 190 in a single forward pass, without requiring a noise input or an iterative denoising process. In some embodiments, the trained multimodal generative model 186 comprises a single regression head that predicts all output values simultaneously from the adapted representation 184 (e.g., a linear layer that maps the 1×H adapted representation to a vector of all output values in a single forward pass).

In some embodiments (e.g., in the muti-head variant), the trained multimodal generative model 186 comprises a plurality of regression heads (e.g., a respective regression head for each of one or more of the output values) that each independently predicts a respective output value from the adapted representation 184. For example, when the output values include signature scores for gene groups, a respective regression head may be provided for each gene group for which a signature score is to be predicted. In the multi-head variant, a (e.g., each) respective regression head independently maps its own adapted representation (e.g., produced by its own respective gated attention network, as described above) to a respective output value, enabling each output value to be predicted from a fully independent pathway through the model. This allows the model to learn a separate prediction function for a respective output value, rather than sharing a single prediction function across all output values, which may improve prediction accuracy for output values that depend on distinct morphological features of the tissue image. The multi-head variant may provide per-output spatial attention and interpretability. For example, this may enable the generation of a separate spatial attention heatmap for a respective predicted output value, as described herein with respect to FIGS. 11A-11C.

In some embodiments, the trained multimodal generative model 186 may be trained by minimizing a loss between the predicted output values and corresponding ground truth output values. For example, this may include minimizing a mean squared error (MSE) loss, a mean absolute error (MAE) loss, or a combination thereof. In some embodiments, a sample-weighted loss is used during training. For example, sample-weighted loss may be used to up-weight samples with rare or extreme output values. Additional details of example trained multimodal generative models used to directly generate output values from tissue image representations are described herein including at least with respect to FIG. 10A.

The one or more output values 190 generated by the trained multimodal generative model 186 may include any suitable output values that can be predicted from the adapted representation 184 of the tissue image. In some embodiments, the one or more output values 190 may be one or more continuous values. The number output values may include any suitable number of output values, such as any number between 1 and 1,000. In some embodiments, the output values 190 comprise signature scores for a plurality of gene groups. Examples of gene groups are described herein including at least in Table 2. The output value(s) 190 may include signature scores for any suitable number of gene groups such as, for example, a number of gene groups between 1 and 100. For example, the output values may include a signature score for a single gene group, at least 3 gene groups, at least 5 gene groups, at least 10 gene groups, at least 20 gene groups, at least 30 gene groups, at least 40 gene groups, at least 50 gene groups listed in Table 2. In some embodiments, the output values 190 may additionally or alternatively comprise a classification, prediction, or score for the subject derived from the adapted representation 184. It should be appreciated that the output values 190 are not limited to any particular type of output value, as aspects of the technology described herein are not limited in this respect.

The output values 190 may be used to perform one or more downstream tasks, as described herein with respect to act 129 of illustrative technique 100.

### Example System

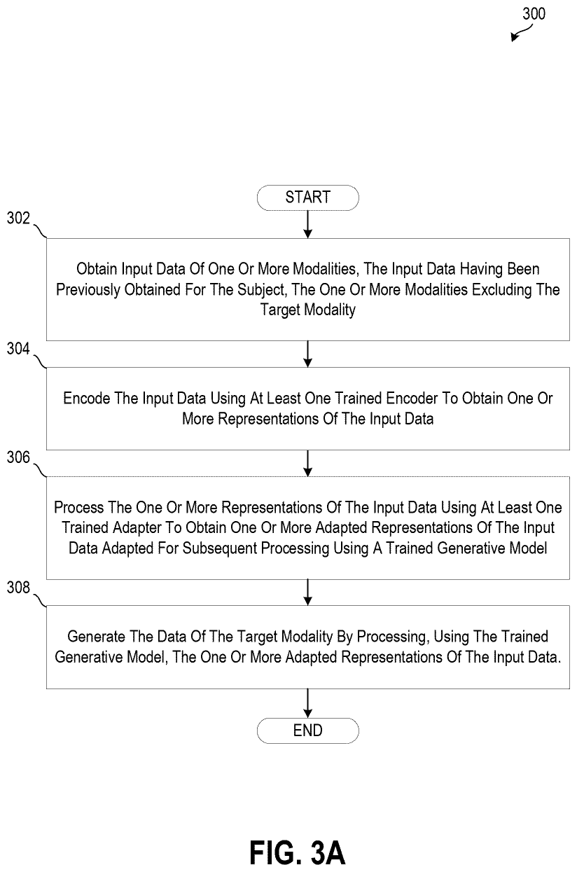

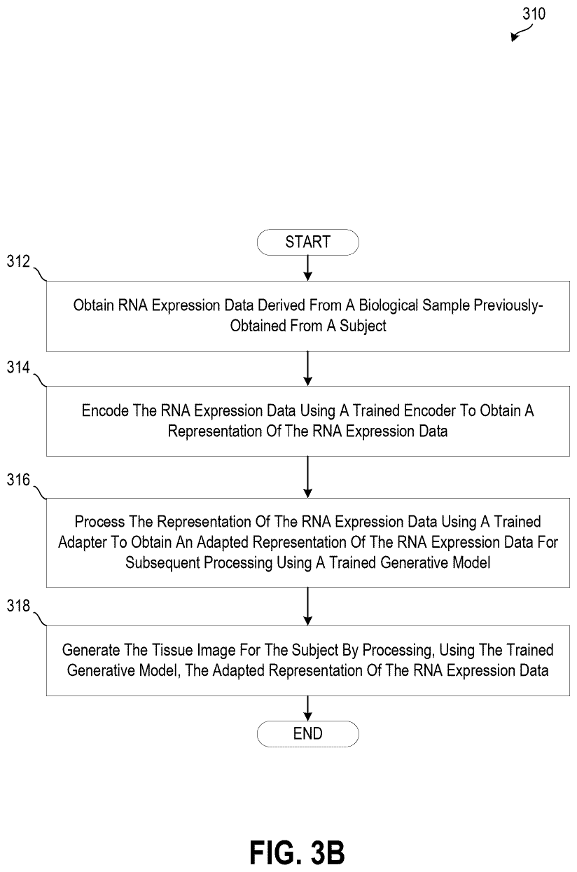

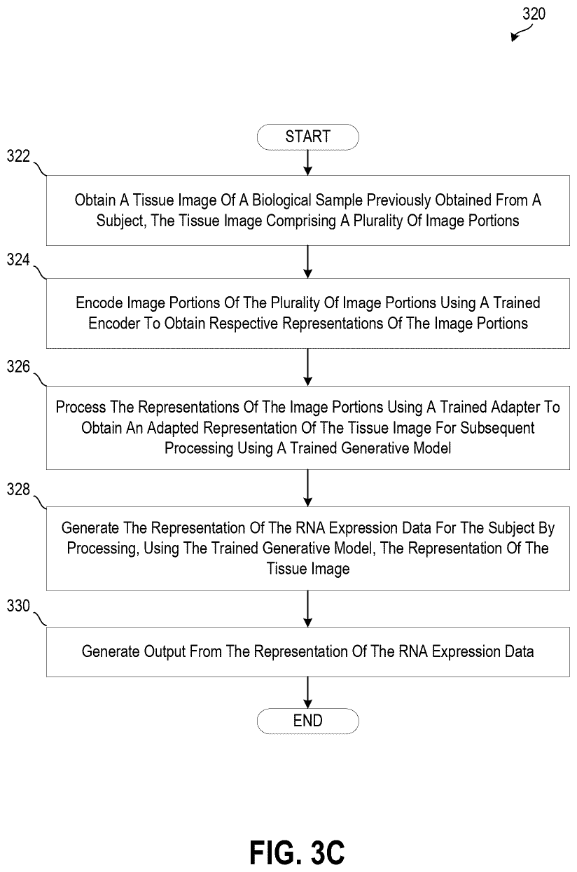

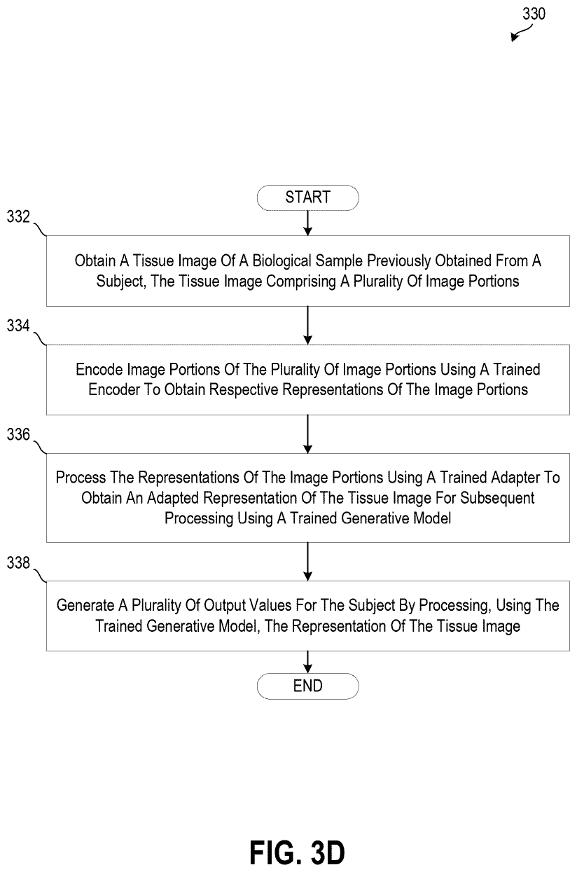

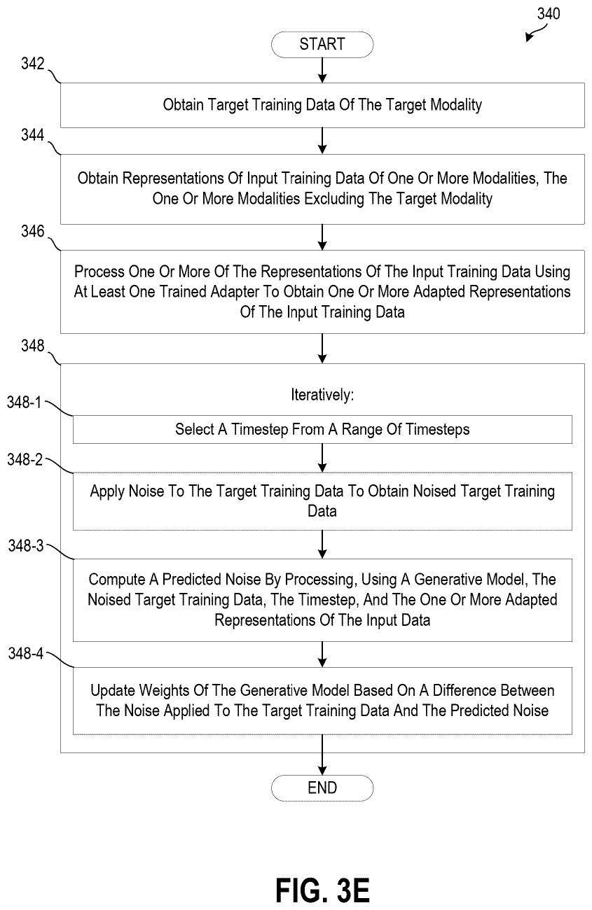

FIG. 2 is a block diagram of an example system 200 for generating data of a target modality for a subject, according to some embodiments of the technology described herein. System 200 includes computing device(s) 210 and data measurement platform(s) 290. The computing device(s) 210 may be configured to have software 250 execute thereon to perform various functions in connection with generating data of a target modality from input data of one or more other modalities. Software 250 includes a plurality of modules. A module may include processor-executable instructions that, when executed by at least one computer hardware processor, cause the at least one computer hardware processor to perform one or more functions of the module. Such modules are sometimes referred to herein as “software modules,” each of which includes processor-executable instructions configured to perform one or more acts of one or more processes, such as the processes described herein with respect to FIGS. 3A-3E.

The computing device(s) 210 may be operated by one or more users 220. The user(s) 220 may provide input specifying processing to be performed by the computing device(s) 210. For example, the user input may include: input specifying the input data to be processed, the target modality for which data is to be generated, the processing to be performed on the input data, and/or the downstream tasks to be performed using the generated data. User(s) 220 may provide input by uploading one or more files, interacting with a user interface module 285, and/or using any other suitable technique for providing input, as aspects of the technology described herein are not limited in this respect.

As shown in FIG. 2, the components of system 200 are configured to communicate with one another to perform the functions described herein. The software modules 255, 260, 265, 270, 275, 280, and 285 execute on computing device(s) 210 and communicate with one another by passing data and instructions between modules in sequence. For example, the output of a module may serve as input to the next module in the processing pipeline. The data store(s) 215, 220 serve as a central repository for data and trained models. The data store(s) 215, 220 may be written to and/or read from by one or more of the software modules. The computing device(s) 210 may communicate with user(s) 220 via the user interface module 285. The computing device(s) 210 may communicate with other components of system 200 via one or more communication networks (e.g., the Internet, a local area network, or any other suitable network) or via a direct connection, as aspects of the technology described herein are not limited in this respect.

In some embodiments, the software 250 may be accessed by user(s) 220 and/or external systems in any suitable way. For example, in some embodiments, the software 250 may be accessed via an application programming interface (API) that exposes one or more endpoints through which external systems and applications may submit input data, specify processing parameters, retrieve generated data, and/or retrieve reports generated by the report generation module 275. In some embodiments, the software 250 may be accessed via the user interface module 285 that provides a graphical user interface through which user(s) 220 may interact with the software 250 directly. In some embodiments, the software 250 may be deployed as a cloud-based service (e.g., on a public cloud infrastructure) and accessed remotely by user(s) 220 and/or external systems via the Internet.

In some embodiments, the software 250 may be deployed on-premises on computing device(s) 210 operated by a healthcare institution or research organization, and accessed locally by user(s) 220 within that institution. In some embodiments, the software 250 may be accessed programmatically (e.g., by an electronic health record system, a laboratory information management system (LIMS), or any other suitable clinical or research information system) via an API or other suitable interface, enabling automated submission of input data and retrieval of generated data and reports as part of a clinical or research workflow. It should be appreciated that the software 250 may be accessed in any suitable way, as aspects of the technology described herein are not limited to any particular access method or deployment configuration.

As shown in FIG. 2, software 250 may include a data pre-processing module 255, an encoding module 260, an adapter module 265, a data generation module 270, a report generation module 275, and a machine learning model training module 280, among others. In operation, input data of one or more modalities obtained from data measurement platform(s) 290, user(s) 220, and/or subject data store(s) 215 is processed by software 250 to generate data of the target modality. First, the input data may be processed by the data pre-processing module 255, then by the encoding module 260 and adapter module 265, and finally by the data generation module 270. The generated data may be provided to the report generation module 275 for report generation.

The data pre-processing module 255 may be configured to preprocess input data obtained for a subject. The data pre-processing module 255 may preprocess the input data prior to encoding using the encoding module 260. For example, the data pre-processing module 255 may be configured to normalize, filter, segment, tile, augment, tokenize, and/or perform feature extraction on the input data, depending on the type of modality and the requirements of the trained encoder. For example, the data pre-processing module 255 may be configured to normalize RNA expression data to obtain TPM values, apply a log transformation, and/or filter out non-coding transcripts or genes with low median expression. Additionally or alternatively, the data pre-processing module 255 may be configured to divide a tissue image into a plurality of image portions (e.g., non-overlapping tiles), apply stain normalization, and/or filter out image portions depicting insufficient tissue content. The data pre-processing module 255 may obtain the input data from user(s) 220, subject data store(s) 215, and/or data measurement platform(s) 290. In some embodiments, the data pre-processing module 255 may be used to perform act 160 of the process shown in FIG. 1C to generate image portions from a tissue image. Examples of preprocessing steps that may be performed by the data pre-processing module 255 are described herein including at least in the section entitled “Data Modalities.”

The encoding module 260 may be configured to encode at least some of the (pre-processed) input data using at least one trained encoder to obtain one or more representations of the input data. The encoding module 260 may be configured to encode input data of multiple modalities using a single trained encoder for all input data modalities. Alternatively, the encoding module 260 may be configured to encode input data of multiple modalities using a respective trained encoder for each input modality. For example, the encoding module 260 may be configured to encode RNA expression data using a trained autoencoder (e.g., a LIAE or VQ-VAE) to obtain a representation of the RNA expression data. Additionally or alternatively, the encoding module 260 may be configured to encode tissue image portions using a trained vision transformer (e.g., a DINOv2-trained ViT or a pre-trained foundation model) to obtain a representation of each of at least some of the image portions. The encoding module 260 may obtain the trained encoder(s) from the ML model data store(s) 220 and/or the machine learning model training module 280. The encoding module 260 may obtain the (pre-processed) input data from the data measurement platform(s) 290, user(s) 220, subject data store 215, and/or data pre-processing module 255. In some embodiments, the encoding module 260 may be used to perform act 304 of the process 300 shown in FIG. 3A, act 314 of the process 310 shown in FIG. 3B, act 324 of the process 320 shown in FIG. 3C, and/or act 334 of the process 330 shown in FIG. 3D. Examples of encoders and encoding techniques are described herein including at least with respect to FIGS. 1A-1D, 6G-6I, 8B, and 9C.

The adapter module 265 may be configured to process the representation(s) of the input data produced by the encoding module 260 using at least one trained adapter to obtain one or more adapted representations of the input data suitable for use as input to, or for conditioning, a trained multimodal generative model. For example, the adapter module 265 may use a trained adapter configured to map a representation of data of an input modality (e.g., RNA expression data) to a representation (e.g., a sequence of tokens) compatible with the cross-attention layers of a denoising diffusion model. Additionally or alternatively, the adapter module 265 may use a trained adapter configured to aggregate a set of representations (e.g., a variable-length set of tile-level representations) into a single adapted representation (e.g., a single adapted representation of a tissue image). The adapter module 265 may obtain the trained adapter(s) from the ML model data store(s) 220 and/or the machine learning model training module 280. The adapter module 265 may obtain the representation(s) of the input data from the encoding module 260 and/or subject data store 215. In some embodiments, the adapter module 265 may be used to perform act 306 of the process 300 shown in FIG. 3A, act 316 of the process 310 shown in FIG. 3B, act 326 of the process 320 shown in FIG. 3C, and act 336 of the process 330 shown in FIG. 3D. Examples of trained adapters are described herein including at least with respect to FIGS. 1A-1D, 6J, 8C, and 10B.

The data generation module 270 may be configured to generate data of the target modality by processing the adapted representation(s) produced by the adapter module 265 using a trained multimodal generative model. For example, when the trained multimodal generative model is a trained denoising diffusion model, the data generation module 270 may be configured generate data of the target modality by iteratively denoising a noise input conditioned on the adapted representation(s). When the trained multimodal generative model is a trained regression model, the data generation module 270 may be configured to use the trained regression model to directly map the adapted representation(s) to one or more output values for the subject. The data generation module 270 may obtain the trained multimodal generative model from the ML model data store(s) 220 and/or the machine learning model training module 280. The data generation module 270 may provide the generated data to the subject data store(s) 215 and/or the report generation module 275 for further processing or storage. In some embodiments, the data generation module 270 may be used to perform act 308 of the process 300 shown in FIG. 3A, act 318 of the process 310 shown in FIG. 3B, act 328 of the process 320 shown in FIG. 3C, and act 338 of the process 330 shown in FIG. 3D. Examples of trained multimodal generative models and data generation techniques are described herein including at least with respect to FIGS. 1A-1D, 6A-6B, 8A, 8D, and 10A.

The report generation module 275 may be configured to generate one or more reports based on the generated data and/or the input data. In some embodiments, the one or more reports include the generated data of the target modality (e.g., generated tissue images, generated RNA expression data, predicted signature scores, etc.). The generated data may be provided together with the input data from which they were generated, providing a comprehensive multimodal profile of the subject.

In some embodiments, the one or more reports include one or more downstream analysis results derived from the generated data. This may include, for example, a characterization of the subject's tumor microenvironment, a predicted treatment response, a predicted molecular subtype, a cohort identified for the subject, a treatment identified for the subject, an indication of disease progression, or any other suitable information. In some embodiments, the one or more reports include spatial attention heatmaps generated from per-tile attention weights produced by the adapter module 265, identifying morphological regions of a tissue image most informative for the generated output values. In some embodiments, the one or more reports include recommendation(s) for a healthcare provider (e.g., a recommendation to administer a particular therapeutic agent, a recommendation to enroll the subject in a clinical trial, or a recommendation to perform additional molecular testing.

The user interface module 285 may be configured to generate a user interface (e.g., a graphical user interface (GUI)) through which user(s) 220 may provide input and view information generated by software 250. For example, the user(s) 220 may view reports generated by the report generation module 275, upload input data for processing, specify the target modality for which data is to be generated, and/or specify the downstream tasks to be performed using the generated data. In some embodiments, the user interface module 285 may be a webpage or web application accessible through an Internet browser. In some embodiments, the user interface module 285 may generate a GUI of an app executing on a user's mobile device. In some embodiments, the user interface module 285 may generate a number of selectable elements through which a user may interact (e.g., dropdown lists, checkboxes, text fields, or any other suitable element.

The machine learning model training module 280 may be configured to train one or more machine learning models for use in connection with generating data of a target modality. For example, the machine learning model training module 280 may be configured to train one or more encoders to encode data of one or more input modalities. Additionally or alternatively, the machine learning model training module 280 may be configured to train one or more trained adapters to produce adapted representations of the input data that are suitable for use by a trained multimodal generative model. Additionally or alternatively, the machine learning model training module 280 may be configured to train a multimodal generative model to generate data of a target modality from adapted representations of input data of one or more other modalities. Additionally or alternatively, the machine learning model training module 280 may be configured to train a decoder to decode representations to their original data space. In some embodiments, the machine learning model training module 280 may be used to perform the process 340 shown in FIG. 3E. Examples of machine learning models and techniques for training same are described herein including at least with respect to FIGS. 1A-1D and in the section entitled “Training.” The machine learning model training module 280 may provide the trained machine learning model(s) to the ML model data store(s) 220 for storage.

As shown in FIG. 2, example system 200 additionally includes subject data store(s) 215 and ML model data store(s) 220. Each of the data stores 215, 220 includes any suitable type of data store (e.g., a flat file, a database system, a multi-file data store, etc.) and may store data in any suitable format, as aspects of the technology described herein are not limited in this respect. The subject data store(s) 215 may include one or more data stores configured to store data obtained for or generated for a subject. Additionally or alternatively, the subject data store(s) 215 may be configured to store reports generated by the report generation module 275, and/or intermediate results produced by one or more of the modules 255, 260, 265.

The ML model data store(s) 220 may include one or more data stores configured to store one or more trained machine learning models. For example, the ML model data store(s) 220 may be configured to store trained encoder(s), trained adapter(s), and/or trained multimodal generative model(s). In some embodiments, the ML model data store(s) 220 store parameter values and weights for trained machine learning model(s). When the stored trained machine learning model(s) are loaded and used (e.g., by modules 260, 265, and 270) the parameter values and weights of the trained machine learning model are loaded and stored in memory using at least one data structure.

As shown in FIG. 2, example system 200 may additionally include data measurement platform(s) 290. The data measurement platform(s) 290 may include any of the data measurement platform(s) described herein, including at least with respect to FIG. 1A, or any other suitable data measurement platform that may be used to obtaining input data of one or more modalities.

### Example Processes for Generating Data Using a Multimodal Generative Mode

FIG. 3A is a flowchart of an illustrative process 300 for generating data of a target modality for a subject from one or more input modalities, according to some embodiments of the technology described herein. One or more (e.g., all) of the acts of process 300 may be performed automatically by any suitable computing device(s). For example, the act(s) may be performed by a laptop computer, a desktop computer, one or more servers, in a cloud computing environment, computing device(s) 110 as described herein including at least with respect to FIG. 1A, computing device(s) 210 as described herein including at least with respect to FIG. 2, computing system 1200 as described herein including at least with respect to FIG. 12, and/or in any other suitable way, as aspects of the technology described herein are not limited in this respect.

At act 302, input data of one or more modalities is obtained for the subject. The input data may be obtained or may have been previously obtained for the subject. For example, the input data may be retrieved from a data store, or may be obtained by measurement the input data using one or more data measurement platforms. Examples of input data and techniques for obtaining same are described herein including at least with respect to FIG. 1A. For example, the input data may include input data 108.

At act 304, the input data is encoded using at least one trained encoder to obtain one or more representations of the input data. In some embodiments, the input data of the one or more modalities is encoded using at least one trained encoder. In some embodiments, the at least one trained encoder includes a single trained encoder. In some embodiments, multiple trained encoders are used to encode the input data. For example, the input data may be encoded using a respective trained encoder for each of the modalities. Examples of encoders and encoding techniques are described herein including at least with respect to FIG. 1A and the section entitled “Training.” For example, the at least one trained encoder may include the trained encoders 1-N shown at act 112 in FIG. 1A.

At act 306, the one or more representations of the input data are processed using at least one trained adapter to obtain one or more adapted representations of the input data. In some embodiments, a single trained adapter is used to process the adapted representations of data of the one or more modalities. In some embodiments, multiple trained adapters are used to process the representations. For example, the multiple trained adapters may include a respective trained adapter for each modality. Examples of trained adapters and techniques for processing representations using same are described herein including at least with respect to FIG. 1A and the section entitled “Training.” For example, the at least one trained adapter may include the trained adapters 1-N shown at act 116 in FIG. 1A.

At act 308, data of the target modality is generated by processing, using the trained multimodal generative model, the one or more adapted representations of the input data. Examples of trained multimodal generative models and techniques for generating data of a target modality using same are described herein including at least with respect to FIG. 1A and the section entitled “Training.” For example, the trained multimodal generative model may include the model 124 shown in FIG. 1A.

FIG. 3B is a flowchart of an illustrative process 310 for generating a tissue image from RNA expression data for a subject, according to some embodiments of the technology described herein. Process 310 is an example of process 300 shown in FIG. 3A, in which the target modality is tissue image data and the input data includes RNA expression data. One or more (e.g., all) of the acts of process 310 may be performed automatically by any suitable computing device(s). For example, the act(s) may be performed by computing device(s) 110 as described herein including at least with respect to FIG. 1A, computing device(s) 210 as described herein including at least with respect to FIG. 2, computing system 1200 as described herein including at least with respect to FIG. 12, and/or in any other suitable way, as aspects of the technology described herein are not limited in this respect.

At act 312, RNA expression data is obtained for the subject. The RNA expression data may be obtained or may have been previously obtained for the subject. For example, the RNA expression data may be retrieved from a data store or may be obtained by measuring the RNA expression data using one or more data measurement platforms, such as a sequencing platform. Examples of RNA expression data and techniques for obtaining same are described herein including at least with respect to FIG. 1B. For example, the RNA expression data may include RNA expression data 134 shown in FIG. 1B.

At act 314, the RNA expression data is encoded using a trained encoder to obtain a representation of the RNA expression data. In some embodiments, the trained encoder is an autoencoder, such as a latent image autoencoder (LIAE) or a vector-quantized variational autoencoder (VQ-VAE), trained to encode RNA expression data into a latent representation. Examples of encoders used to encode RNA expression data and techniques for training same are described herein including at least with respect to FIGS. 1B, 6G, 6H, 6I, and the section entitled “Training.”

At act 316, the representation of the RNA expression data is processed using a trained adapter to obtain an adapted representation of the RNA expression data. In some embodiments, the trained adapter comprises one or more linear projection layers and one or more non-linear activation layers configured to map the representation of the RNA expression data to a sequence of tokens compatible with the conditioning mechanism of the trained multimodal generative model. Examples of trained adapters used to process representations of RNA expression data are described herein including at least with respect to FIGS. 1B and 6J.

At act 318, a tissue image is generated for the subject by processing, using a trained multimodal generative model, the adapted representation of the RNA expression data. In some embodiments, the trained multimodal generative model is a denoising diffusion model that iteratively denoises the noise input conditioned on the adapted representation to generate the tissue image or a representation thereof, which may be subsequently decoded into the tissue image by a decoder. Examples of trained multimodal generative models used to generate tissue images from RNA expression data are described herein including at least with respect to FIGS. 1B, 6A, 6B, and the section entitled “Training.” For example, the trained multimodal generative model may include model 144 shown in FIG. 1B.

FIG. 3C is a flowchart of an illustrative process 320 for generating, from a tissue image, a representation of RNA expression data and an output based on the representation of RNA expression data for a subject, according to some embodiments of the technology described herein. Process 320 is an example of process 300 shown in FIG. 3A, in which the target modality is RNA expression data and the input data includes tissue image data. One or more (e.g., all) of the acts of process 320 may be performed automatically by any suitable computing device(s). For example, the act(s) may be performed by computing device(s) 110 as described herein including at least with respect to FIG. 1A, computing device(s) 210 as described herein including at least with respect to FIG. 2, computing system 1200 as described herein including at least with respect to FIG. 12, and/or in any other suitable way, as aspects of the technology described herein are not limited in this respect.

At act 322, a tissue image is obtained for the subject. The tissue image may be obtained or may have been previously obtained for the subject. For example, the tissue image may be retrieved from a data store or medical imaging archive, or may be obtained by measuring the tissue image using one or more data measurement platforms, such as an imaging platform. Examples of tissue images and techniques for obtaining same are described herein including at least with respect to FIG. 1C. For example, the tissue image may include tissue image 158 shown in FIG. 1C.

In some embodiments, the tissue image obtained at act 322 includes a plurality of image portions. For example, the tissue image may be pre-processed to obtain the plurality of image portions such as, for example, by dividing the tissue image into a plurality of smaller tiles. In some embodiments, image portions depicting less than a threshold amount of tissue are excluded from subsequent processing. Examples of techniques for generating image portions from a tissue image are described herein including at least with respect to act 160 shown in FIG. 1C.

At act 324, the image portions are encoded using a trained encoder to obtain respective representations of the image portions. In some embodiments, the trained encoder is a vision transformer trained using a self-supervised learning method, or a pre-trained histopathology foundation model used as a fixed feature extractor. Examples of encoders used to encode tissue image portions and techniques for training same are described herein including at least with respect to FIGS. 1C, 8B, 9C, and the section entitled “Training.”

At act 326, the representations of the image portions are processed using a trained adapter to obtain an adapted representation of the tissue image. In some embodiments, the trained adapter is configured to perform multiple instance learning to aggregate the variable-length set of tile-level representations into a single fixed-size slide-level adapted representation using a gated attention mechanism. Examples of trained adapters used to process representations of tissue image portions are described herein including at least with respect to FIGS. 1C and 8C.

At act 328, a representation of RNA expression data is generated for the subject by processing, using a trained multimodal generative model, the adapted representation of the tissue image. In some embodiments, the trained multimodal generative model is a denoising diffusion model that iteratively denoises the noise input conditioned on the adapted representation to generate a latent representation of the RNA expression data. Examples of trained multimodal generative models used to generate representations of RNA expression data from tissue images are described herein including at least with respect to FIGS. 1C, 8A, 8D, and the section entitled “Training.” For example, the trained multimodal generative model may include model 172 shown in FIG. 1C.

At act 330, an output is generated from the representation of RNA expression data. In some embodiments, the output comprises decoded RNA expression data obtained by processing the representation of the RNA expression data using a trained decoder. In some embodiments, the output comprises one or more signature scores for one or more gene groups derived from the representation of the RNA expression data or the decoded RNA expression data. Examples of outputs that may be generated from the representation of RNA expression data are described herein including at least with respect to FIG. 1C.

FIG. 3D is a flowchart of an illustrative process 330 for generating, from a tissue image, a plurality of output values for a subject, according to some embodiments of the technology described herein. Process 330 is an example of process 300 shown in FIG. 3, in which the target modality is output values (e.g., signature scores) and the input modality is tissue image data. One or more (e.g., all) of the acts of process 330 may be performed automatically by any suitable computing device(s). For example, the act(s) may be performed by computing device(s) 110 as described herein including at least with respect to FIG. 1A, computing device(s) 210 as described herein including at least with respect to FIG. 2, computing system 1200 as described herein including at least with respect to FIG. 12, and/or in any other suitable way, as aspects of the technology described herein are not limited in this respect.

At act 332, a tissue image is obtained for the subject. The tissue image may be obtained or may have been previously obtained for the subject. For example, the tissue image may be retrieved from a data store or medical imaging archive, or may be obtained by measuring the tissue image using one or more data measurement platforms, such as an imaging platform. Examples of tissue images and techniques for obtaining same are described herein including at least with respect to FIG. 1D, and the same description applies here. For example, the tissue image may include tissue image 158 shown in FIG. 1D.

In some embodiments, the tissue image obtained at act 332 includes a plurality of image portions. For example, the tissue image may be pre-processed to obtain the plurality of image portions such as, for example, by dividing the tissue image into a plurality of smaller tiles. In some embodiments, image portions depicting less than a threshold amount of tissue are excluded from subsequent processing. Examples of techniques for generating image portions from a tissue image are described herein including at least with respect to act 160 shown in FIG. 1C.

At act 334, the image portions are encoded using a trained encoder to obtain respective representations of the image portions. Examples of encoders used to encode tissue image portions are described herein including at least with respect to FIG. 1D, and the same description applies here.

At act 336, the representations of the image portions are processed using a trained adapter to obtain an adapted representation of the tissue image. In some embodiments, the trained adapter comprises a feature extraction network and at least one gated attention network configured to aggregate the variable-length set of tile-level representations into a single fixed-size adapted representation of the tissue image using attention-weighted aggregation. Examples of trained adapters used in this embodiment are described herein including at least with respect to FIGS. 1D, 10B, 10C, and 10D.

At act 338, a plurality of output values is generated for the subject by processing, using a trained multimodal generative model, the adapted representation of the tissue image. In some embodiments, the trained multimodal generative model is a regression model comprising one or more regression heads that map the adapted representation to one or more continuous output values. In some embodiments, the output values comprise signature score(s) for one or more gene groups. Examples of trained multimodal generative models used to generate output values directly from tissue image representations are described herein including at least with respect to FIGS. 1D, 10A, and 11A, and in the section entitled “Training.”

### Training

The techniques described herein may involve training one or more machine learning models. For example, the techniques may involve training one or more encoders, one or more decoders, one or more adapters, and/or one or more multimodal generative models. Examples of such models are described herein including at least with respect to FIGS. 1A-1D.

In some embodiments, some or all of the models are trained jointly. For example, the adapter(s) and generative model may be trained jointly while holding the encoder weights fixed. Joint training may involve training multiple models simultaneously as a single unified system (e.g., with gradients flowing through all models during each training step). In such embodiments, loss may be computed at the output of the pipeline and backpropagated through the models simultaneously. This approach allows the jointly trained models to be optimized jointly for the same objective.

Additionally or alternatively, some or all of the model(s) may be trained separately on their own objectives, without coordination between models during training. For example, the encoder may be trained on a reconstruction objective, the adapter may be trained on an adaption objective, and the generative model may be trained on a generation objective, with each model trained to convergence before being combined with the others.

In some embodiments, one or more of the models are initialized from pre-trained weights. For example, as described herein, the backbone (e.g., neural network backbone) of a generative model may be initialized from the weights of a pre-trained model (e.g., diffusion model). As another example, the encoder may be initialized from the weights of a pre-trained encoder (e.g., a foundation model). In some embodiments, training a pre-trained model may include fine-tuning the pre-trained model on domain-specific training data.

In some embodiments, one or more of the model(s) may be trained using a contrastive or alignment learning objective. For example, the encoder(s) and/or adapter(s) may be trained using contrastive learning. A contrastive learning objective encourages representations derived from different modalities but corresponding to the same subject to be more similar to one another than representations derived from different subjects. By encouraging representations of different modalities from the same subject to occupy related positions in the shared latent space, contrastive training may improve the ability of the trained multimodal generative model to generate data of a target modality from available input modalities, particularly in settings where one or more input modalities are missing, since a well-aligned latent space allows the generative model to more accurately infer the characteristics of the missing modality from the available ones. Contrastive training may be performed before, during, or jointly with training of the trained multimodal generative model. Contrastive learning may be applied to any suitable pair or combination of modalities for which paired training data is available. It should be appreciated that contrastive or alignment training of the encoder(s) and/or adapter(s) is optional, and that aspects of the technology described herein are not limited to any particular training objective.

The specific training approach(es) used for any one model may depend on the availability of training data (e.g., paired training data), the architecture of the model, the size of the available data set(s), and whether suitable pre-trained components are available, among other considerations. Examples of training techniques for specific models are described in more detail below.

### Training: Encoders

Aspects of the technology described herein relate to processing encoding input data using a trained encoder to obtain one or more representations of the input data.

The trained encoder(s) may be trained using any approach suitable for the architecture of the encoder. For example, the encoder(s) may be obtained by training an encoder-decoder pair, by training an encoder using a self-supervised or semi-supervised objective, or by using a pre-trained encoder, optionally with further finetuning on domain-specific data.

In some embodiments, a trained encoder is obtained from a trained encoder-decoder pair. An encoder-decoder pair refers to a pair comprising an encoder configured to encode input data into a compact latent representation and a decoder configured to reconstruct the input data from that latent representation. Training the encoder as part of an encoder-decoder pair may encourage the encoder to produce a compact latent representation that captures sufficient information to reconstruct the input.

The encoder-decoder pair may be trained by optimizing the decoder to accurately reconstruct the input data from the latent representation produced by the encoder. For example, the encoder-decoder pair may be trained by minimizing a reconstruction loss such as mean squared error or mean absolute error between the input data and the reconstructed data. In some embodiments, the encoder-decoder pair is trained using a combination of reconstruction loss and regularization loss. For example, a KL divergence loss may be used in the case of a VAE, an L2 regularization loss may be used in the case of an LIAE, or a commitment loss and codebook loss may be used in the case of a VQ-VAE.

Optimization may be performed using any suitable optimizer such as an adaptive movement estimation (Adam) optimizer, AdamW, or a stochastic gradient descent (SGD) optimizer, for example. The optimizer may use any suitable learning rate, such as a learning rate between 1e-5 and 1e-2, for example. The encoder-decoder pair may be trained for any suitable number of epochs, such as between 5 and 5,000 epochs. Additionally, the encoder-decoder pair may be trained using any suitable batch size, such as between 60 and 5,000. In some embodiments, augmentations may be applied to the latent representation during training to improve robustness. For example, augmentations may include latent dropout, Gaussian noise injection, random erasing, and/or random rotation. Additionally or alternatively, augmentations may be applied to the input data during training. For example, data may be randomly dropped to simulate missing measurements and improve encoder robustness.

Once trained, the encoder may be extracted from the pair and used, optionally with its weights held fixed, to encode input data. In some embodiments, the decoder of the encoder-decoder pair may also be used downstream. For example, the decoder may be used to decode a representation of the target modality generated by the trained multimodal generative model into the data of the target modality, as described herein. Examples of encoders obtained from encoder-decoder pairs include the LIAE encoder and VQ-VAE encoder described herein with respect to FIGS. 6G, 6H, and 6I, which are trained to encode RNA expression data into a compact latent representation from which the expression data can be reconstructed.

In some embodiments, a trained encoder is obtained using a self-supervised learning objective that does not require a paired decoder. Self-supervised approaches may be broadly categorized into teacher-student frameworks, contrastive learning methods, redundancy-reduction methods, and masked prediction objectives. Though, some frameworks combine multiple self-supervised objectives. Encoders trained using such self-supervised objectives may be suitable for modalities for which large corpora of unlabeled data are available, such as histopathological images. An example of such an encoder described herein is the vision transformer (ViT) used to encode tissue image portions, as described herein with respect to FIGS. 8B and 9C.

Some self-supervised approaches include a teacher-student framework, in which the teacher and student networks each process different augmented views of the same image, and the student is trained so that its embedding of its view matches the teacher's embedding of its view. The teacher's weights are maintained as an exponential moving average of the student's weights across training iterations. Examples of teacher-student frameworks that may be used to train a self-supervised encoder include Self-Distillation with No Labels (DINO), DINOv2, DINOv3 Bootstrap Your Own Latent (BYOL), iBOT, and Mean Teacher, among others. DINO is described by Caron, M., et al. (“Emerging properties in self-supervised vision transformers.” Proceedings of the IEEE/CVF international conference on computer vision. 2021.); DINOv2 is described by Oquab, M., et al. (“Dinov2: Learning robust visual features without supervision.” ar Xiv preprint ar Xiv: 2304.07193 (2023).); DINOv3 is described by Simeoni, O., et al. (“Dinov3.” ar Xiv preprint ar Xiv: 2508.10104 (2025).); BYOL is described by Grill, Jean-Bastien, et al. (“Bootstrap your own latent-a new approach to self-supervised learning.” Advances in neural information processing systems 33 (2020): 21271-21284.); iBOT is described by Zhou, J., et al. (“ibot: Image bert pre-training with online tokenizer.” ar Xiv preprint ar Xiv: 2111.07832 (2021).); Mean Teacher is described by Tarvainen, A. and Valpola, H. (“Mean teachers are better role models: Weight-averaged consistency targets improve semi-supervised deep learning results.” Advances in neural information processing systems 30 (2017).), each of which is incorporated by reference herein

Other self-supervised approaches include contrastive learning method and masked prediction objectives. For example, using a contrastive learning objective, an encoder may be trained to maximize agreement between representations of augmented views of the same sample while minimizing agreement between representations of different samples. Examples of contrastive learning frameworks that may be used to train a self-supervised encoder include Momentum Contrast (MoCo), Simple Framework for Contrastive Learning (SimCLR), Barlow Twins, and VICReg, among others (some of these methods do not require explicit negative pairs). MoCo is described by He, K., et al. (“Momentum contrast for unsupervised visual representation learning.” Proceedings of the IEEE/CVF conference on computer vision and pattern recognition. 2020.); SimCLR is described by Chen, Ting, et al. (“A simple framework for contrastive learning of visual representations.” International conference on machine learning. PmLR, 2020.); Barlow Twins is described by Zbontar, J., et al. (“Barlow twins: Self-supervised learning via redundancy reduction.” International conference on machine learning. PMLR, 2021.); and VICReg is described by Bardes, Adrien, et al. “Vicreg: Variance-invariance-covariance regularization for self-supervised learning.” ar Xiv preprint arXiv: 2105.04906 (2021).), each of which is incorporated by reference in its entirety. Using a masked prediction objective, an encoder may be trained to predict masked portions of the input from the unmasked portions. Examples of masked prediction frameworks that may be used to train a self-supervised encoder include Masked Autoencoders (MAE), Bidirectional Encoder Representations from Transformers (BERT), and Joint-Embedding Predictive Architecture (JEPA), among others.

In some embodiments, a pre-trained encoder is used as a trained encoder without further fine-tuning. For example, a large model pre-trained on a large and diverse corpus of data using self-supervised or other objectives, referred to herein as a pre-trained foundation model, may be used as a fixed feature extractor without any additional training. Pre-trained foundation models may be particularly well-suited as encoders when the pre-training corpus is sufficiently large and diverse that the resulting representations generalize well to the target domain and task. Examples of pre-trained foundation models that may be used as encoders for tissue image data include H-Optimus-0, H-Optimus-1, Hibou, UNIv2, and Virchow, among others, as described herein with respect to FIG. 1A.

In some embodiments, a pre-trained encoder is further fine-tuned on domain-specific data prior to use. Fine-tuning involves initializing the encoder from the pre-trained weights and then continuing to train the encoder, either fully or partially, on a domain-specific dataset. For example, a pre-trained foundation model may be fine-tuned on a dataset of histopathological images from a specific cancer type or imaging protocol to adapt its representations to that specific domain. Fine-tuning may be performed using any suitable objective, such as a supervised classification loss, a self-supervised loss, or a reconstruction loss, depending on the availability of labeled data and the requirements of the downstream task. In some embodiments, only a subset of the encoder's layers are fine-tuned while the remaining layers are held fixed, a technique sometimes referred to as partial fine-tuning or layer-wise fine-tuning. For example, only the final few transformer blocks of a vision transformer may be fine-tuned while the earlier blocks are held fixed, preserving the general-purpose representations learned during pre-training while adapting the higher-level representations to the target domain. In some embodiments, parameter-efficient fine-tuning methods are used to adapt a pre-trained encoder to a new domain without updating all of its parameters. For example, low-rank adaptation (LoRA) may be used to fine-tune a pre-trained encoder by adding small trainable low-rank matrices to selected layers of the encoder, enabling adaptation with a small number of additional trainable parameters relative to the full size of the encoder.

In some embodiments, the encoder weights are held fixed during training of the adapter(s) and/or multimodal generative model(s), such that the encoder serves as a fixed feature extractor. For example, the encoder may first be trained on a first set of training data, then the generative model(s) (and optionally the adapter(s)) may be trained on a second set of training data different from the first set of training data. For example, the second set of training data may include paired training data (e.g., pairs of one or more input data modalities and a target data modality). By contrast, the first set of training data may include additional or alternative data, including data other than paired training data, which enables the encoder to be trained on more training data than may be available for the generative model(s).

In other embodiments, the encoder is trained jointly with one or more other components of the system, such as the trained adapter(s) or the trained multimodal generative model. The choice of whether to fix or jointly train the encoder weights may depend on the size of the available training dataset, the complexity of the encoder, and the degree to which the encoder's pre-trained representations are already suited to the target task.

### Training: Decoders

Aspects of the technology described herein relate to using one or more trained decoders to decode representations of input and/or target data modalities.

In some embodiments, the trained decoder(s) are trained jointly with their corresponding encoder(s) as part of an encoder-decoder pair, as described above with respect to training encoders. Once the encoder-decoder pair has been trained, the decoder may be extracted and used independently to decode representations of the target modality generated by the trained multimodal generative model into data in the original data space of the target modality. For example, the decoder component of a trained LIAE may be used to decode a latent representation of RNA expression data generated by a denoising diffusion model into a TPM expression vector, as described herein with respect to FIG. 8A. Similarly, the decoder component of a pre-trained VAE, such as the VAE from Stable Diffusion 1.5, may be used to decode a latent representation of a tissue image generated by a denoising diffusion model into a tissue image, as described herein with respect to FIG. 6A. In some embodiments, the decoder weights are held fixed during training of the adapter(s) and generative model(s). In other embodiments, the decoder may be fine-tuned jointly with other components of the system.

Training: Adapters Aspects of the technology described herein relate to processing representation(s) of input data using a trained adapter to obtain one or more adapted representations of the input data.

In some embodiments, the trained adapter(s) are trained jointly with the trained multimodal generative model by backpropagating the generative model's training loss through both the generative model and the adapter simultaneously, while the encoder weights are held fixed. In some embodiments, the adapter is trained after the encoder and generative model backbone have been pre-trained separately, serving as a learned bridge between the two pre-trained components. In some embodiments, the adapter, encoder, and generative model are all trained jointly end-to-end. The choice of training approach may depend on the availability of paired training data, the complexity of the adapter, and whether suitable pre-trained encoder and generative model components are available.

The specific training procedure may vary depending on the type of adapter used. In some embodiments, the adapter is a projection-based adapter, such as an embedding projector, that maps a representation of the input data to a sequence of tokens compatible with the conditioning mechanism of the trained multimodal generative model. In such embodiments, the embedding projector may be trained jointly with the supplementary cross-attention layers of the generative model, while the weights of the encoder and the pre-trained generative model backbone are held fixed, as described herein with respect to FIGS. 6A-6J. In some embodiments, classifier-free guidance is used during training of the embedding projector, such that the conditioning input is randomly dropped with a suitable probability so that the model learns both a conditional and an unconditional version of the conditioning function.

In some embodiments, the adapter is a gated attention pooler configured to aggregate representations of multiple samples (e.g., image portions) into a single conditioning vector for use by a denoising diffusion model, as described herein with respect to FIG. 8C. In such embodiments, the gated attention pooler may be trained jointly with the denoising diffusion model while the encoder weights are held fixed, with gradients flowing through both the denoising diffusion model and the gated attention pooler simultaneously. In some embodiments, a sampling strategy is used during training, in which a fixed number of samples is sampled with replacement from each sample set (e.g., tissue image) at each training iteration, providing implicit data augmentation and ensuring a fixed sequence length across sample sets within a batch regardless of the actual number of samples per set.

In some embodiments, the adapter comprises a feature extraction network and at least one gated attention network configured to aggregate sample-level representations into an adapted representation for use by a regression model, as described herein with respect to FIGS. 10B, 10C, and 10D. In such embodiments, the feature extraction network and gated attention network(s) may be trained jointly with the regression model in an end-to-end training procedure, with gradients flowing through all components simultaneously. As with the gated attention pooler described above, a sampling strategy may be used during training to provide implicit data augmentation and ensure a fixed sequence length across sample sets within a batch.

Optimization may be performed using any suitable optimizer such as Adam, AdamW, or a stochastic gradient descent (SGD) optimizer, for example. Any suitable learning rate may be used, such as, for example, a value between 1e-5 and 1e-3. Any suitable learning rate schedule may be used such as, for example, as a cosine decay schedule with linear warmup.

### Training: Regression Models

As described herein, in some embodiments, the trained multimodal generative model is a regression model that is trained to directly predict one or more output values for a subject. The regression model may be trained by minimizing a loss between the predicted output values and corresponding ground truth output values derived from paired training data. For example, the training loss may be a mean squared error (MSE) loss, a mean absolute error (MAE) loss, a Huber loss, or any weighted combination thereof, or any other suitable training loss, as aspects of the technology described herein are not limited in this respect. In some embodiments, the training loss is computed jointly over all output values. In some embodiments, a sample-weighted loss is used during training. For example, the loss may be computed as a weighted average over training samples, with higher weights assigned to samples with rare or extreme output values, to reduce the influence of class imbalance or distributional skew on the learned model.

The regression model may be optimized using any suitable optimizer, such as AdamW, Adam, or SGD, with any suitable learning rate, such as a value between 1e-5 and 1e-2. The regression model may be optimized using any suitable learning rate schedule, such as cosine decay or linear warmup, for example.

The model may be trained with any suitable batch size, such as between 10 and 200 slides per batch. In some embodiments, the training data is augmented during training. For example, a fixed number of training samples (e.g., image portions) may be sampled with replacement from each sample set (e.g., image). The number of sampled training samples may include between 100 and 1,000 samples per set, or any other suitable number, as aspects of the technology described herein are not limited in this respect. Examples of regression model training are described herein including at least with respect to FIG. 10A.

### Training: Denoising Diffusion Models

Aspects of the technology described herein relate to processing representation(s) of input data using a trained adapter to obtain one or more adapted representations of the input data.

In some embodiments, the trained multimodal generative model is a denoising diffusion model trained to generate data of a target modality conditioned on adapted representations of input data of one or more other modalities. During training, the forward diffusion process progressively adds noise to a training sample of the target modality at each of a series of timesteps according to a noise schedule, producing a sequence of increasingly noisy versions of the training sample. The noise may be any suitable type of noise, such as Gaussian noise, for example. The noise schedule may be any suitable variance schedule, such as a linear or cosine-based variance schedule. The total number of training timesteps T may be any suitable number, such as between 50 and 2,000. The neural network backbone, such as a UNet or transformer, is trained to predict the noise added at each timestep by minimizing a loss between the predicted and true noise. The loss may be any suitable loss function, such as mean squared error or mean absolute error. In some embodiments, training follows the denoising diffusion probabilistic model (DDPM) framework. For example, noise may be sampled according to the DDPM forward noise process.

The neural network backbone may be conditioned on the adapted representation(s) of the input data via cross-attention layers, so that noise prediction is guided by the available input modality data. In some embodiments, the neural network backbone is pre-trained and its weights are at least partially frozen (e.g., fixed) during training, such that only the conditioning-related components are updated. For example, only supplementary cross-attention layers of the denoising diffusion model may be updated during training, as described herein with respect to FIGS. 6A-6J. In other embodiments, the neural network backbone is not pre-trained.

In some embodiments, classifier-free guidance is used during training by randomly dropping the conditioning input with a suitable probability. For example, the conditioning input may be dropped with a probability of between 5% and 25%, so that the model learns both a conditional and an unconditional version of the denoising function.

In some embodiments, the denoising diffusion model operates in a compressed latent space defined by a pre-trained encoder-decoder pair, in which case the training samples of the target modality are first encoded into the latent space before the forward diffusion process is applied.

The denoising diffusion model may be trained using any suitable optimizer. For example, the optimizer may be AdamW, Adam, or SGD. Any suitable learning rate may be used, such as a value between 1e-5 and 1e-3. Any suitable learning rate schedule may be used, such as a cosine decay schedule with linear warmup. Any suitable batch size may be used, such as between 16 and 8,192, optionally with gradient accumulation to achieve a larger effective batch size. In some embodiments, mixed precision training may be used to reduce memory requirements and improve training speed. For example, brain float 16 (bf16) or float 16 (fp16) mixed precision training may be used. An illustrative process for training a denoising diffusion model is described herein with respect to FIG. 3E. Additional examples of denoising diffusion model training are described herein with respect to FIGS. 6A-6J, and 8A-8K.

FIG. 3E is a flowchart of an illustrative process 340 for training a multimodal generative model (e.g., a denoising diffusion model) to generate data of a target modality, according to some embodiments of the technology described herein. In some embodiments, process 340 may be performed by computing device(s) 110, for example using the machine learning model training module 280 described herein with respect to FIG. 2.

As shown in FIG. 3E, process 340 begins at act 342, where target training data of the target modality is obtained. The target training data comprises training samples of the target modality that serve as the ground truth against which the multimodal generative model's outputs are evaluated during training. For example, when training the generative model to generate tissue images, the target training data may include tissue images. In some embodiments, the target training data is paired with input training data. For example, a paired sample may include (i) a training sample of the target modality obtained from a subject, and (ii) training samples of one or more input modalities obtained for that same subject. For example, the training data may include a tissue image for the subject (e.g., the target modality) and RNA expression data (e.g., input modality) for the subject The paired training data enables the multimodal generative model to learn the relationship between the input and target modalities. The target training data may be obtained using any of the techniques for obtaining data of the input and target modalities described herein, including at least with respect to FIG. 1A.

At act 344, representations of input training data of one or more modalities is obtained for the subject. In some embodiments, the representations of the input training data are obtained by encoding the input training data using a trained encoder at act 344. In other embodiments, the input training data may have been previously encoded, and the representations may be retrieved from a data store. In embodiments that include encoding the input training data, the encoder weights may be held fixed during process 340. In other embodiments, the encoder is trained jointly with the adapter and generative model during process 340.

As shown in FIG. 3E, process 340 continues at act 346, where the representations of the input training data are processed using at least one trained adapter to obtain one or more adapted representations, as described herein with respect to FIG. 1A and the Training Adapters section above. In some embodiments, the adapter is trained jointly with the multimodal generative model during process 340, meaning that the adapter weights are updated at act 348-4 together with the weights of the multimodal generative model. In other embodiments, the adapter weights are held fixed during process 340.

As shown in FIG. 3E, process 340 continues at act 348, which is performed iteratively. At act 348-1, a timestep t is selected from the range {1, . . . , T} using any suitable sampling strategy. For example, the timestep may be sampled uniformly from the range {1, . . . , T}.

At act 348-2, noise is applied to a training sample of the target modality according to a noise schedule to obtain a noised training sample at timestep t. For example, Gaussian noise corresponding to the selected timestep t may be applied according to a linear or cosine-based variance schedule.

At act 348-3, a predicted noise is computed by processing, using the multimodal generative model, the noised training sample, the timestep, and the adapted representations of the input training data. For example, the neural network backbone of the denoising diffusion model may process the noised training sample, the timestep embedding, and the adapted representations to produce a predicted noise estimate. In some embodiments, the adapted representation is provided to the neural network backbone as a conditioning input via cross-attention layers. In a cross-attention layer, the adapted representation(s) are linearly projected to produce the keys and values used for generating the output of the cross-attention layer. For example, the intermediate feature representations of the neural network backbone at a given layer may serve as the queries (Q), while the adapted representation of the input data is linearly projected to produce the keys (K) and values (V). Attention weights may be computed as the softmax of the scaled dot product between the queries and keys (e.g., softmax (QK\hat{ }T/√d), where d is the dimensionality of the keys). The output of the cross-attention layer may be the weighted sum of the values, determined using the computed attention weights.

At act 348-4, the weights of the multimodal generative model and, optionally, the weights of the adapter, are updated based on a loss between the predicted noise and the true noise. For example, the loss may be mean squared error or mean absolute error, backpropagated using any suitable optimizer and learning rate schedule.

In some embodiments, at act 348-4, weights of all cross-attention layers are updated. In other embodiments, weights of only some of the cross-attention layers are updated. For example, when the generative model includes a pre-trained backbone, it may include two types of cross-attention layer: (i) original cross-attention layers that were trained during pre-training, and (ii) supplementary cross-attention layers for incorporating the adapted representations of the input data. In such embodiments, only weights of the supplementary cross-attention layers may be updated.

Acts 348-1 through 348-4 are repeated until convergence. For example, convergence may be determined based on stabilization of the training loss, achievement of a target validation performance, or by reaching a maximum number of training iterations.

### Multimodal Foundation Model Examples and Applications

FIGS. 4A-1-4C provide examples of using a multimodal foundation model to generate data of a target modality from multiple input data modalities, according to some embodiments of the technology described herein. For example, the data of the target modality may be generated according to technique 100 shown in FIG. 1A.

FIGS. 4A-1-4A-2 and FIGS. 4B-1-4B-3 illustrate examples of the overall multimodal framework described herein, showing how data from multiple modalities at the patient level, immune system level, tissue level, and molecular level may each be preprocessed, encoded into modality-specific embeddings, adapted (e.g., aggregated and conditioned), and then processed by a trained multimodal generative model to produce outputs suitable for interpretation, characterization of biological context, and downstream prediction tasks. FIGS. 4A-1 and 4A-2 provides a high-level overview of the framework, while FIGS. 4B-1-4B-3 provides a more detailed example of the specific data types, preprocessing steps, encoder architectures, and embedding dimensionalities that may be used for each modality. As shown in both figures, the modality-specific representations (e.g., embeddings) produced by the encoders may be adapted (e.g., aggregated and conditioned) before being provided to a trained multimodal generative model that generates an output representation of the target modality.

The following paragraphs describe each of the example modalities illustrated in FIGS. 4A-1-4B-3 in more detail, including the types of data the modality may encompass, the techniques by which such data may be obtained, and how the data may be preprocessed, encoded, and adapted for use by the trained multimodal generative model. It should be appreciated that the specific modalities, architectures, and preprocessing steps described herein are merely examples, and that any suitable modality, architecture, and preprocessing approach may be used, as aspects of the technology described herein are not limited in this respect. Additional examples of data types that may serve as input data of one or more modalities, and the platforms and file formats from which they may be obtained, are listed in Table 1.

### Histopathology Images

In some embodiments, the input data includes histopathology images, such as hematoxylin and eosin (H&E) stained slides and related brightfield tissue images. Histopathology images are examples of tissue images, as described herein. Such images may provide information about tissue morphology, tumor architecture, stromal composition, necrosis, invasion patterns, and/or local immune context. Histopathology images may be obtained by processing a tissue specimen obtained from a subject, for example by sectioning, staining, and mounting the specimen on a glass slide and digitizing the slide using a whole-slide image scanner at a suitable magnification. Additional imaging platforms that may be used to obtain histopathology images include fluorescence microscopes, multiplexed imaging systems, and spatial transcriptomics imaging systems, as described herein with respect to FIG. 1C.

Because whole-slide images may be gigapixel-scale, the encoder may operate using a tile-based strategy in which slides are segmented to exclude background and artifacts, tessellated into tiles, optionally normalized for stain variation, and encoded using a self-supervised vision backbone. In some embodiments, the backbone is a vision transformer trained using a teacher-student framework, a masked image model, a contrastive encoder, or a related self-supervised architecture. Examples of suitable self-supervised approaches include DINO, DINOv2, DINOv3,

JEPA, masked autoencoders, MoCo, Barlow Twins, VICReg, and related methods. As a result of the encoding, each of at least some (e.g., all) of the tiles may be converted into a respective latent embedding capturing local morphology, texture, tissue composition, and architectural context. Tile embeddings may then be adapted (e.g., aggregated) by attention-based pooling, hierarchical transformers, graph-based aggregation, or spatial token fusion to produce either a global slide-level embedding or a structured token grid preserving spatial relationships across the tissue.

### RNA Expression Data

In some embodiments, the input data includes RNA expression data. RNA expression data may provide a quantitative measurement of transcriptional state, including tumor biology, immune activation, stromal programs, lineage identity, pathway activity, and/or treatment-associated perturbations. RNA expression data may be obtained from a biological sample, such as a tissue biopsy or blood sample, using a sequencing platform. For example, bulk RNA sequencing may be performed on a tissue sample or on blood cells to produce counts, TPM (Transcripts Per Million), FPKM (Fragments Per Kilobase of transcript per Million mapped fragments), CPM (Counts per million), or related abundance measures at the gene or transcript level. RNA expression data may also be obtained from single-cell RNA sequencing platforms, spatial transcriptomics platforms, or RNA panels, as described herein. Additional examples of RNA expression data and techniques for obtaining same are described herein including at least in the section entitled “RNA Expression Data”

Preprocessing the RNA expression data may include normalization, log transformation, batch correction, filtering of low-information genes, selection of highly variable genes, pathway-aware grouping, and/or construction of gene sets or signatures. The encoder may transform the expression profile into a latent representation using a variational autoencoder, vector-quantized VAE, masked transformer, encoder-decoder transformer, multilayer perceptron, convolutional architecture, graph-based encoder, or similar learned compression model. In some embodiments, the encoder is trained with gene masking, dropout augmentation, permutation-invariant objectives, or denoising reconstruction losses to improve robustness and encourage the model to learn higher-order biological dependencies. The output embedding may represent the whole sample as a single latent vector or as a structured set of tokens corresponding to genes, pathways, gene modules, cell-state programs, or other transcriptional units.

### DNA-Based Genomic Variation Data

In some embodiments, the input data includes DNA-based genomic variation data, including somatic mutations, germline variants, insertions, deletions, and/or related sequence alterations. The DNA-based genomic variation data may provide a representation of tumor drivers, inherited susceptibility, resistance mechanisms, clonal evolution, and pathway-level perturbation. DNA-based genomic variation data may be obtained from whole-genome sequencing, whole-exome sequencing, or targeted sequencing panels applied to a biological sample such as a tumor biopsy or blood draw, and may be represented in VCF, MAF, or comparable formats.

Preprocessing may include alignment, recalibration, quality filtering, annotation, somatic-germline distinction, and harmonization of variant-level metadata. In some embodiments, the encoder operates on genomic variation using one or more of the following strategies: direct variant tokenization; sequence-context embedding of altered loci; gene-level aggregation of mutation burden; mutational signature decomposition; topic-model or factorization approaches; autoencoder-based compression of sparse mutation matrices; or network-informed representations that estimate the functional influence of a variant on pathway activity or downstream cellular phenotypes. In some embodiments, variants are encoded as tokens that include mutation type, genomic context, affected gene, predicted consequence, zygosity, clonality, or cancer relevance. A transformer or related attention-based model may learn weighted mutation-set embeddings that emphasize informative drivers and co-occurring events.

### Copy Number Variation and Structural Variant Data

In some embodiments, the input data includes copy number variations (CNVs) and structural variants (SVs), including, for example, amplifications, deletions, rearrangements, translocations, and/or gene fusions. The CNVs and SVs may provide orthogonal genomic information not fully captured by small-variant calls. CNV and SV data may be obtained from sequencing-based or array-based pipelines applied to tumor or germline samples and may be represented as segmented copy-number profiles, gene-level copy states, breakpoint calls, fusion calls, or structural event annotations.

Preprocessing may include segmentation, log-ratio normalization, ploidy correction, purity-aware adjustment, breakpoint detection, and harmonization of missing values. The encoder may transform these inputs into latent embeddings using regional tokenization across chromosomal segments, gene-set or cytoband aggregation, transformer-based sequence models over ordered genomic loci, graph-based encoders over rearrangement structure, or autoencoder-based compression of genome-wide copy profiles. CNV and SV embeddings may be used independently or merged with mutation embeddings to produce a richer genomic representation for multimodal integration.

### Cell-Free RNA, Cell-Free DNA, and Cell-Free Methylation Data

In some embodiments, the input data includes liquid biopsy modalities such as cell-free RNA (cfRNA), cell-free DNA (cfDNA), and cell-free methylation data. Such data may capture systemic and circulating disease-associated signals. It may be useful for longitudinal monitoring, early response assessment, minimal residual disease detection, and tissue-free inference of tumor and immune states. These modalities may be obtained from a blood sample, for example by isolating and sequencing cell-free nucleic acids from plasma or serum using next-generation sequencing, targeted sequencing, or methylation sequencing platforms.

Cell-free RNA may be processed similarly to bulk RNA expression data but with additional normalization and denoising steps appropriate for low-input, fragmented, and/or high-noise transcripts. Cell-free DNA may include variant calls, copy-number patterns, fragmentation profiles, methylation signals, and/or tumor fraction estimates. Cell-free methylation may provide epigenetic information reflecting tissue of origin, cell-state composition, and/or tumor-associated regulatory states. Encoders for liquid biopsy data may include sequence-based encoders, autoencoders, transformers, denoising models, fragment-length distribution encoders, methylation-profile encoders, or fused multimodal plasma encoders. In some embodiments, cfRNA embeddings share the same or a related latent space as tissue RNA encodings, enabling tissue-blood alignment.

### Single-Cell Omics Data

In some embodiments, the input data includes single-cell omics data, including single-cell RNA sequencing, single-cell DNA sequencing, single-cell assay for transpose-accessible chromatin (ATAC) sequencing, or multimodal single-cell assays. Such data may provide cellular-resolution information regarding transcriptional heterogeneity, clonal structure, lineage composition, differentiation trajectories, and cell-state transitions. Single-cell omics data may be obtained from a biological sample, such as a tissue biopsy or blood sample, using single-cell sequencing platforms such as 10× Genomics Chromium, Smart-seq, or related droplet-based or plate-based single-cell sequencing systems.

Preprocessing may include demultiplexing, alignment, quality control, doublet filtering, normalization, batch correction, feature selection, cell annotation, and/or construction of cell-by-feature matrices. In some embodiments, the input is first summarized into biologically meaningful units such as cell-type proportions, metacells, cluster signatures, clonal states, or neighborhood-level features. The encoder may operate at the cell level, cluster level, or sample level. In some embodiments, the encoder encodes individual cells using one or more transformers, graph neural networks, variational autoencoders, or manifold-learning architectures. The encoded representations may be adapted (e.g., using a trained adapter) by aggregating across cells to create sample-level embeddings. The resulting embeddings may preserve within-sample heterogeneity, major cell populations, rare but important subpopulations, trajectory structure, and tumor-immune ecosystem composition.

### Metabolomic and Proteomic Data

In some embodiments, the input data includes metabolomic and proteomic data. Such data may provide functional biochemical readouts of pathway utilization, energetic state, nutrient dependencies, metabolic stress, protein abundance, signaling activity, and/or pathway-level functional biology. Metabolomic and proteomic data may be obtained using mass spectrometry, immunoassays, targeted panels, Luminex-based platforms, and/or related assay systems applied to biological samples such as blood plasma, serum, tissue, or urine, and may be represented as intensities, quantified concentrations, peak tables, or derived pathway-level features.

Preprocessing may include normalization, batch correction, peak alignment, missing-value handling, feature aggregation, and/or transformation to stabilize distributions. Encoders may comprise principal-component layers, autoencoders, variational autoencoders, transformer-based numeric encoders, graph-based pathway encoders, or multitask denoising models. Proteomic and metabolomic embeddings may be represented as continuous latent vectors or as pathway-specific or analyte-specific tokens. The embeddings may be useful for linking upstream molecular alterations to downstream functional phenotypes and for enabling multimodal reconstruction of pathway activity, inflammatory state, or treatment-induced biochemical changes.

### Flow Cytometry, CyTOF, and Cell Count Data

In some embodiments, the input data includes flow cytometry, mass cytometry (CyTOF), and/or related cell count data. Such data may provide measurements of immune composition and phenotypic cell-state distributions at single-cell resolution. Flow cytometry and CyTOF data may be obtained by staining a blood or tissue sample with fluorescently labeled or metal-conjugated antibodies targeting surface and intracellular markers, and acquiring single-cell measurements using a flow cytometer or mass cytometer. Raw inputs may include FCS files or derived cell-level marker matrices.

Preprocessing may include compensation, gating, quality control, normalization, marker harmonization, clustering, and/or generation of population-level abundance or phenotype summaries. In some embodiments, the encoder operates on processed cell-population frequencies, marker-expression summaries, or hierarchical immune-subset counts. In other embodiments, the encoder operates directly on high-dimensional single-cell distributions using deep set models, transformers, density encoders, or graph-based representations. The resulting embeddings may capture proportions of T-cell subsets, myeloid populations, NK-cell states, memory phenotypes, activated or exhausted compartments, and other immune features.

### Radiology Data

In some embodiments, the input data includes radiological imaging data, including CT, MRI, PET, and/or multimodal combinations thereof. Such data may provide macroscopic information about tumor burden, lesion distribution, vascularization, tissue density, necrosis, metabolic activity, and/or temporal response dynamics. Radiological images may be obtained using clinical imaging systems such as CT scanners, MRI scanners, or PET scanners. They may be stored in DICOM or equivalent volumetric formats. Inputs may include multiple sequences, timepoints, or fused imaging channels.

Preprocessing may include resampling, intensity normalization, co-registration, segmentation of lesions or regions of interest, and/or cropping around anatomically relevant volumes. Encoders may include 3D convolutional networks, volumetric vision transformers, masked volumetric autoencoders, or hybrid architectures that preserve both local and global three-dimensional structures. The encoder output may represent one lesion, multiple lesions, or an integrated subject-level radiological phenotype. The encoder output may be aligned with pathology, blood biomarkers, and longitudinal clinical outcomes.

### Immunohistochemistry and Multiplexed Spatial Imaging Data

In some embodiments, the input data includes immunohistochemistry (IHC), multiplex immunofluorescence, imaging mass cytometry, and/or related spatial assays. Such data may provide protein-level and/or cell-neighborhood information characterizing the spatial organization of tumor, immune, and stromal compartments. IHC data may be obtained by staining a tissue section with antibodies targeting specific protein markers and imaging the stained section using a brightfield or fluorescence scanner. Multiplexed spatial imaging data may be obtained using platforms such as cyclic immunofluorescence (CyCIF), CODEX, or imaging mass cytometry systems that capture multiple protein markers simultaneously from the same tissue section.

For standard IHC, preprocessing may include color deconvolution, stain quantification, segmentation of positive and negative regions, and/or extraction of intensity or localization features. For multiplexed imaging, preprocessing may include channel alignment, denoising, cell segmentation, marker co-expression extraction, and/or construction of local spatial descriptors. Encoders may operate on image patches, cell-level feature tables, neighborhood graphs, or combinations thereof. The resulting embeddings may capture protein expression intensity, subcellular localization, cell phenotype composition, tumor-immune interfaces, immune exclusion patterns, tertiary lymphoid structures, stromal barriers, and/or other spatially organized tissue states.

### Methylation Profile Data

In some embodiments, the input data includes DNA methylation and related epigenetic profiles. Such data may provide information regarding cell of origin, lineage commitment, regulatory state, silencing programs, tumor subtype, and/or disease evolution. DNA methylation data may be obtained using methylation arrays such as the Illumina EPIC array, whole-genome bisulfite sequencing, and/or targeted methylation sequencing assays. The DNA methylation data may be represented as beta values, region-level methylation summaries, and/or CpG-site matrices. Preprocessing may include normalization, batch correction, probe filtering, region aggregation, and/or imputation of missing measurements. Encoders may include autoencoders, transformer-based numeric encoders, region-aware tokenization models, or pathway-level epigenetic aggregators. The resulting embeddings may reflect cell-type composition, regulatory programs, tissue-of-origin patterns, therapy-associated epigenetic changes, and/or tumor-specific methylation structure. The resulting embeddings may be aligned with transcriptomics, cell-free DNA, and clinical data to support multimodal biological inference.

### Immune Repertoire Data

In some embodiments, the input data includes T-cell receptor (TCR) and B-cell receptor (BCR) sequencing data. Such data may provide information regarding adaptive immune diversity, clonality, antigen-driven expansion, repertoire architecture, and/or immune engagement. TCR and BCR sequencing data may be obtained from a blood or tissue sample using bulk or single-cell immune repertoire sequencing platforms, such as the 10× Genomics Chromium immune profiling system or Adaptive Biotechnologies immunoSEQ. Such data may be represented as clonotype tables, V(D) J annotations, CDR3 sequences, clone frequencies, chain-pairing information, isotype information, and/or repertoire diversity metrics.

Preprocessing may include alignment, clonotype clustering, frequency normalization, sequence cleaning, and/or extraction of diversity or convergence statistics. Encoders may operate directly on receptor sequences, clonotype frequency distributions, diversity summaries, and/or learned receptor-language-model embeddings. The output may be a repertoire-level latent vector, a clonotype token set, or a hierarchical embedding representing clonal abundance, diversity, expansion, and sequence-motif structure. These embeddings may be integrated with transcriptomic, cytometry, pathology, and clinical features to model adaptive immune response as part of the broader multimodal framework.

### Clinical Tabular Metadata

In some embodiments, the input data includes structured clinical data. It may include, for example, demographics, disease stage, performance status, treatment exposure, comorbidities, laboratory results, vital signs, and/or response endpoints. Such data may be obtained from electronic health record systems, clinical trial databases, laboratory information management systems, and/or structured case report forms. It may include numerical variables, ordinal variables, categorical variables, timestamps, treatment-line information, and/or routine laboratory values such as complete blood count, chemistries, liver function tests, and inflammatory markers.

Preprocessing may include normalization, one-hot or learned categorical encoding, missing-value handling, temporal alignment, and/or construction of clinically meaningful derived features. Encoders may include embedding tables, multilayer perceptrons, transformer-based tabular encoders, or mixed-type feature encoders. The resulting embeddings may be represented as a single vector or as multiple clinical tokens. The resulting embeddings may be aligned with molecular and imaging embeddings to support multimodal state representation, outcome prediction, and longitudinal disease modeling.

### Textual Clinical Data

In some embodiments, the input data includes unstructured textual data. This may include, for example, clinical notes, pathology reports, radiology reports, discharge summaries, molecular reports, and/or treatment narratives. Such data may be obtained from electronic health record systems, clinical documentation platforms, and/or structured reporting systems. It may contain contextual information that may be absent from structured fields including, for example, disease history, clinician interpretation, response descriptions, adverse event context, and nuanced phenotype information that complements numeric and molecular modalities.

Preprocessing may include document ingestion, format harmonization, section parsing, de-identification, chunking, and/or temporal association with other modalities. Encoders may include biomedical language models, transformer-based clinical language encoders, report-level embedding models, or multimodal vision-language systems when text is paired with image-derived content. The resulting text embeddings may capture latent representations of disease course, pathology descriptors, treatment-related events, radiological impressions, and/or symptom narratives. The resulting embeddings may be fused with structured and molecular data to enrich the subject representation used by the multimodal generative model.

### Additional Modalities

The techniques described herein are not limited to the modalities explicitly described above. Additional modalities may include epigenomic assays such as ChIP-seq, microbiome data, neuroimaging data, wearable sensor streams, radiomic feature sets, digital pathology-derived handcrafted features, functional assays, cytokine panels, spatial transcriptomics, or any biomedical modality capable of being transformed into a learned representation. In each case, the modality-specific encoder may be configured to preserve the modality's native biological signal while generating embeddings that can be aligned within the shared latent space of the multimodal generative model, supporting extensibility as new measurement technologies become available. Examples of data types that may serve as input data and the platforms and formats from which they may be obtained are listed in Table 1.

TABLE 1
Example data modalities.

| | | Representative input |
|---|---|---|
| Data modality/category | Example source platforms | formats |
| Genomics (DNA panels, | Illumina, Ion Torrent, PacBio, | fastq, .bam, .sam, .vcf, |
| WES, WGS, cfDNA) | Oxford Nanopore, MGI | .maf, .gvcf |
| Transcriptomics (RNA | Illumina, 10x Genomics, | fastq, .bam, .rcc, .counts, |
| panels, WTS, cfRNA, | NanoString, Vizgen, NanoString | .tpm, .mtx |
| scRNA, TCR/BCR) | CosMx | |
| Digital pathology/imaging | Leica Aperio, Hamamatsu, | .svs, .ndpi, .qptiff, .dicom, |
| (H&E, IHC, MxIF) | Akoya PhenoImager, Nikon, | .ome.tiff, .ome.zarr, .zarr, |
| | Olympus | .tiff, .tif, .mrxs, .nd2 |
| Cytometry (flow and mass | BD FACS family, Beckman | .fcs, .lmd, .sraw, .acs, .wsp, |
| cytometry) | Coulter CytoFlex, Thermo | .jo, .clr, .c6, .cf1, .ci |
| | Fisher Attune NxT | |
| Spatial-NGS/spatial omics | NanoString, 10x | .dcc, roi, .pkc, .rcc, .vzg, |
| | | .parquet, .h5ad, .inf, .dax |
| Microbiome (16S, 18S, | Illumina MiSeq, Oxford | fastq, .fastq.gz, .biom, .tsv |
| shotgun) | Nanopore | |
| Proteomics | Thermo Fisher Orbitrap Astral, | .fcs, .csv, .xlsx, .raw, .npn |
| | Bruker timsTOF | |
| Metabolomics | SCIEX 7500+, Agilent 6495D | .mzML, .mzXML, .mgf |
| | LC/TQ | |
| Epigenomics | Illumina, 10x Genomics | .fastq, .bam, .bed, .bigWig |
| Clinical metadata | Epic, Oracle Health, Medidata | json, xml, sas7bdat, .hl7 |
| | Rave, REDCap, Veeva | |

FIGS. 5A-5F provide examples of applications of the techniques for generating data of a target modality using a trained multimodal generative model.

FIG. 5A illustrates an example of using the trained multimodal generative model to simulate the effects of specific biological alterations on a subject's multimodal biological profile, according to some embodiments of the technology described herein. As shown in FIG. 5A, the process begins with a subject's existing multimodal data, including a transcriptomic profile (e.g., expression vector) representing the subject's baseline biological state. A hypothetical biological alteration is then defined (e.g., the upregulation or downregulation of a selected gene signature) to simulate the mechanism of action of a therapeutic agent. The alteration is then applied to the subject's input data to produce a perturbed version of the input. The perturbed input is encoded and processed by a trained adapter to produce an adapted representation, which is used to condition the trained multimodal generative model to generate data of the target modality corresponding to the perturbed biological state, such as a histology image. The simulated state may be validated by direct comparison to the real state. For example, the comparison may be performed by contrasting RNA expression heatmaps and visually inspecting the synthesized histology images (e.g., 1024×1024 pixels) against the original patient tissue images. Model performance may be quantitatively gauged by metrics such as the Fréchet inception distance (FID), where lower values indicate a higher-fidelity match to real image distributions.

FIG. 5B illustrates an example of using a trained multimodal generative model to model a subject's trajectory through a high-dimensional biological state landscape, according to some embodiments of the technology described herein. In this approach, a subject's biological condition is conceptualized as a point in a high-dimensional state space defined by multimodal representations of the subject's data across modalities such as transcriptomic profiles, histopathological images, immune cell data, and genomic data. The goal is to identify a path from the subject's current biological state to a more desirable target state by iteratively navigating this landscape. As shown in FIG. 5C, the technique operates as an iterative simulation cycle. The process is initialized with the subject's baseline multimodal data. At each iteration, a limited and controlled modification is introduced to one or more input modalities to create a new input state, the trained multimodal generative model is used to generate the complete multimodal data profile corresponding to the modified input, and the resulting output state is evaluated to determine whether it has moved closer to the desired target state. This cycle is repeated, introducing new modifications at each step, until the simulated state converges on the desired outcome, tracing a trajectory through the biological state space from the subject's starting state to the target state. This trajectory modeling approach may be used to simulate disease progression, identify personalized therapeutic strategies, and determine the most efficient sequence of interventions needed to move a subject's biological state toward a favorable clinical outcome.

FIGS. 5C and 5D illustrates an example of using the trained multimodal generative model to simulate and forecast the systemic influence of a therapeutic agent on a subject, according to some embodiments of the technology described herein. In this approach, the known or hypothesized mechanism of action of a therapeutic agent is translated into a perturbation of the subject's baseline input data to computationally emulate the biological effects of the agent on the subject's transcriptomic profile. For example, the perturbation may be performed by modulating the expression levels of genes associated with the agent's target pathway or immune response. The perturbed input is then encoded and processed by the trained multimodal generative model to synthesize the multimodal patient state corresponding to the simulated drug effect, including predicted data of one or more target modalities such as histopathological images. The resulting generated representations may be projected into a lower-dimensional embedding space that captures clinically relevant biological axes (e.g., axes corresponding to immune activity and stromal or fibrotic biology) enabling visualization of how the subject's biological state is predicted to move from a pre-treatment position to one or more potential post-treatment positions within the biological state landscape, and how those positions relate to biologically and clinically meaningful patient subgroups. This drug influence forecasting approach enables predictive analysis of how a therapeutic agent affects the subject's biological state across multiple modalities, providing insights into the drug's mechanism of action, its effects on the immune microenvironment, and its predicted clinical consequences, which may be used to inform patient stratification and the development of targeted therapeutic strategies.

FIG. 5E illustrates an example of using the trained multimodal generative model to impute missing pathology data from a blood-based RNA assay, according to some embodiments of the technology described herein. Specifically, FIG. 5E shows that tissue-level morphological information, such as histopathological images, may be reconstructed from cell-free RNA profiling data obtained from a blood sample, enabling non-invasive assessment of tissue-level biology without requiring a tissue biopsy and supporting real-time monitoring of disease progression in settings where repeated tissue sampling is infeasible.

FIG. 5F illustrates an example of using the trained multimodal generative model to impute missing genomic data from histopathological image analysis, according to some embodiments of the technology described herein. Specifically, FIG. 5F shows that molecular information, such as genomic biomarker status, may be inferred directly from routinely available H&E tissue images, enabling molecular screening of subjects without requiring sufficient biopsy material for genomic profiling and potentially supporting patient pre-screening for clinical trials where genomic eligibility criteria must be assessed.

### Example: Generating Tissue Image Data from RNA Expression Data

Aspects of the technology described herein relate to generating tissue image data from RNA expression data for a subject. Illustrative techniques for generating tissue image data from RNA expression data are described herein including at least with respect to FIG. 1B and FIG. 3B. FIGS. 6A-7K provide additional details and examples relating to these techniques.

FIGS. 6A-6J illustrate an example technique for generating a tissue image from RNA expression data, and example architectures of the components used to implement that technique, according to some embodiments of the technology described herein. It should be appreciated that the specific architectures and dimensionalities illustrated in FIGS. 6A-6J are merely exemplary, and any suitable architectures and dimensionalities may be used to implement the techniques. For example, the architectures may include additional or alternative layers, and may have different dimensionalities.

FIG. 6A provides an overview of the technique. As shown in FIG. 6A, RNA expression data 601 is first encoded using a trained encoder 602 to obtain a representation 603 of the RNA expression data. The representation 603 is then processed by a trained adapter 604 to obtain an adapted representation 605 of the RNA expression data. The adapted representation 605 is used to condition a denoising diffusion model 607, which iteratively denoises a noise input 606 to generate a representation 609 of the tissue image. The representation 609 is then processed by a decoder 610 to generate the tissue image 611.

As shown in FIG. 6A, the denoising diffusion model 607 comprises original cross-attention layers 608-1 and supplementary cross-attention layers 608-2. In some embodiments, both the original and supplementary cross-attention layers receive the adapted representation 605 of the RNA expression data as a conditioning input. In some embodiments, only the supplementary cross-attention layers received the adapted representation 605 of the RNA expression data as input. In some embodiments, the original cross-attention layers 608-1 are part of a pre-trained backbone (e.g., a pre-trained latent diffusion model), and the supplementary cross-attention layers 608-2 are added as part of an adapter that is trained to inject the RNA expression conditioning signal into the denoising process.

FIG. 6B illustrates an example architecture of the denoising diffusion model 607. As shown in FIG. 6B, the denoising diffusion model 607 has a UNet architecture comprising an initial 2D convolution layer 613, a series of downsampling blocks 614, 615, and 616, residual blocks 617, a bottleneck block 618, residual blocks 619, and a series of upsampling blocks 620, 621, and 622, followed by a final 2D convolution layer 623. The adapted representation 625 of the RNA expression data is provided as a conditioning input to the cross-attention layers of the downsampling, bottleneck, and upsampling blocks, and a time embedding 626 is provided to the residual blocks throughout the network to encode the current diffusion timestep. Skip connections pass the outputs of downsampling blocks to their corresponding upsampling blocks.

FIGS. 6C, 6D, and 6E illustrate the internal architectures of the downsampling, bottleneck, and upsampling blocks of the denoising diffusion model, respectively. As shown in FIG. 6C, each downsampling block 627 comprises two repeated sequences of residual block, self-attention, cross-attention, and feed-forward network layers, followed by a downsampling operation. As shown in FIG. 6D, the bottleneck block 618 comprises a residual block, followed by self-attention, cross-attention, and feed-forward network layers, followed by a second residual block. As shown in FIG. 6E, each upsampling block 631 comprises three repeated sequences of concatenation with skip states, residual block, self-attention, cross-attention, and feed-forward network layers, followed by an upsampling operation. In each case, the cross-attention layers receive the adapted representation of the RNA expression data as a conditioning input, and the residual blocks receive the time embedding.

FIG. 6F illustrates the architecture of the cross-attention layer used within the downsampling, bottleneck, and upsampling blocks. As shown in FIG. 6F, the adapted representation 625 of the RNA expression data may be split into first tokens 638-1 and second tokens 638-2. The first tokens 638-1 may be processed by the original cross-attention layers 640-1, and the second tokens 638-2 may be processed by the supplementary cross-attention layers 640-2. Alternatively, all tokens may be provided to the supplementary cross-attention layers 640-2. When both sets of cross-attention layers are used, the outputs of the two sets of cross-attention layers are summed to produce an output feature representation 642. This dual-branch cross-attention design allows the RNA expression conditioning signal to be injected into both the pre-trained backbone attention layers and the supplementary adapter attention layers simultaneously.

In each branch (original and supplementary), the cross-attention operation is performed as follows. The intermediate feature representations of the neural network backbone at the current layer serve as the queries Q. In the original cross-attention branch, the first tokens 638-1 are linearly projected to produce keys K and values V using the original pre-trained projection weights of the backbone, and standard cross-attention is computed as softmax (QK\hat{ }T/√d)× V. In the supplementary cross-attention branch, the second tokens 638-2 are linearly projected to produce keys Kᵢₚ and values Vᵢₚ using the supplementary adapter projection weights, which comprise two learned linear projection layers (e.g., one mapping the token embeddings to keys of dimensionality compatible with the current backbone block, and one mapping the token embeddings to values of the same dimensionality). Cross-attention is computed as softmax (QKᵢₚ\hat{ }T/√d)× Vᵢₚ. The outputs of the two branches are then summed to produce the output feature representation 642, which is passed to the next layer in the neural network backbone.

FIGS. 6G and 6H illustrate a first example architecture of the trained encoder 602 used to encode the RNA expression data. As shown in FIG. 6G, this encoder, which corresponds to the Latent Image AutoEncoder (LIAE) described herein, takes an RNA expression vector as input and processes it through a series of operations including regularization, a linear projection, normalization, activation, and reshaping to obtain a 2D feature map, which is then processed through a series of residual blocks and 2D convolution layers to produce a representation 603 of the RNA expression data. FIG. 6H illustrates the internal architecture of the residual blocks used in this encoder, each of which comprises 2D convolution layers with normalization and activation, a residual skip connection, and an optional 1D convolution to match input and output channel dimensions when they differ.

FIG. 6I illustrates a second example architecture of the trained encoder 602, corresponding to the Vector-Quantized Variational Autoencoder (VQ-VAE) encoder described herein. As shown in FIG. 6I, this encoder takes a RNA expression vector as input, zero-pads it to a length that is a multiple of 8, and processes it through a series of 1D convolution and activation layers to produce a representation 603 of the RNA expression data. This encoder provides an alternative to the LIAE encoder of FIG. 6G, and the two encoders may be used interchangeably within the technique illustrated in FIG. 6A.

FIG. 6J illustrates an example architecture of the trained adapter 604. As shown in FIG. 6J, the adapter 604 takes the representation 603 of the RNA expression data produced by the encoder as input and processes it through linear projection layers with an intermediate activation, followed by a reshape operation and a normalization layer, to produce an adapted representation 605 of the RNA expression data. This adapted representation (e.g., 8 tokens of embedding dimension 768) is formatted to be compatible with the cross-attention layers of the denoising diffusion model 607, allowing the RNA expression information to be injected into the image generation process via cross-attention conditioning.

As an example, one or more models having the architectures described with respect to FIGS. 6A-6J were trained to generate tissue images from RNA expression data. The models were trained on a dataset of 85,600 RNA expression samples, split 60/40 into training and test sets. Each sample comprises a vector of expression values for up to 20,062 genes, normalized to log-transformed TPM values. Genes were filtered by excluding non-coding transcripts and genes with low median expression. During training, input augmentation was applied by randomly dropping up to 15% of gene values with a 50% probability per sample to simulate missing measurements and improve encoder robustness.

An LIAE encoder, with architecture shown in FIGS. 6G-6H, was trained using an AdamW optimizer with a learning rate of 0.0003, a weight decay of 0.001, a lambda_z regularization weight of 1.0, and a batch size of 1,000, for 1,650 epochs. The training loss comprised a reconstruction MSE loss combined with an L2 regularization term applied to the latent representation. Additional augmentations were applied to the latent representation during training, including latent dropout, Gaussian noise injection, random erasing, and random rotation.

A VQ-VAE encoder-decoder, with the architecture shown in FIG. 6I, was trained on the same dataset of log-transformed TPM expression vectors as the LIAE, with the same 60/40 train/test split. The model was trained using an Adam optimizer with a learning rate of 0.0003, Adam betas of (0.9, 0.999), a codebook size of 1,024, an EMA decay of 0.8, a commitment weight of 0.4, a batch size of 256, and for 11 epochs. The training loss comprised a reconstruction MSE loss combined with a commitment loss and codebook loss. The codebook was active only during training to regularize the latent space. At inference, the codebook was bypassed and the encoder's continuous output was passed directly to downstream stages.

A denoising diffusion model, with architecture shown in FIGS. 6B-6F, was trained to generate 512×512 pixel H&E tissue image tiles conditioned on RNA expression embeddings. The training data comprised paired H&E whole-slide images and RNA expression data, from which 70 tiles of 512×512 pixels at 0.5 microns per pixel were sampled from each slide. RNA expression data was preprocessed by normalizing to log-transformed TPM values and encoding using the trained LIAE encoder to produce a 1,024-dimensional embedding, which was then projected by the trained adapter (FIG. 6J) to produce 8 tokens of embedding dimension 768 for use as a conditioning input to the denoising diffusion model. All UNet backbone weights were held fixed during training. Only the adapter and the supplementary cross-attention layers were updated. The model was trained using an AdamW optimizer with a learning rate of 1e-4 and a weight decay of 1e-2, a batch size of 192, and for 125 epochs, using the standard diffusion noise prediction loss. At inference, a guidance scale of 7.5 and a guidance rescale of 0.0 were used.

At inference, Gaussian noise was used as input to the denoising diffusion model. Representations of tissue images (image embeddings) were generated using the DDIM sampling protocol with 70 steps. The image embeddings were decoded using the decoder portion of a VAE (Stable Diffusion 1.5 VAE decoder) to reconstruct the 512×512 image tiles.

Examples of the generated tissue images are shown in FIGS. 7A-7D. FIG. 7D and FIG. 7E are plots showing the performance of the trained denoising diffusion model in generating tissue images that match corresponding ground truth tissue images. Lower Fréchet inception distance (FID) values suggest that the distribution of generated images closely matches that of the corresponding ground truth images. Additionally, a visual inspection of the generated images reveals high similarity to real histological processes usually observed in clinical conditions, indicating that the techniques may be used to accurately and reliably generate tissue images for a subject from RNA expression data.

FIGS. 7F-7J illustrate examples of the perturbation-based application of the trained multimodal generative model, showing how systematic modification of a subject's RNA expression input produces corresponding changes in the generated tissue images that reflect the expected morphological correlates of the perturbed gene signatures. Specifically, FIGS. 7G-7I show generated tissue images corresponding to progressively increasing levels of the CAF-associated, CD8 T-cell, B-cell, and blood angiogenesis gene signatures respectively. FIG. 7J illustrates how divergent perturbation scenarios from a common starting state produce distinct biological trajectories. Values within the signatures were replaced with the same scalar in the range 0 to 100. The perturbed signatures were processed using the above-described models to generate tissue images. To visualize the morphological transition, signature values were incremented from 0 to 100 in ascending order (e.g., [0, 30, 60, 100]). For each modified vector, DDIM with 70 steps is utilized to generate the corresponding image tiles.

FIG. 7K illustrates an end-to-end example of the full tissue image generation pipeline from RNA expression embeddings to generated and real tissue images.

Generating RNA Expression Data from Tissue Image Data

Aspects of the technology described herein relate to generating RNA expression data from tissue images for the subject. Illustrative techniques for generating RNA expression data from tissue image data are described herein including at least with respect to FIG. 1C and FIG. 3C. FIGS. 8A-9 provide additional details and examples relating to these techniques.

FIGS. 8A-8K illustrate an example technique for generating RNA expression data from a tissue image, and example architectures of the components used to implement that technique, according to some embodiments of the technology described herein. It should be appreciated that the specific architectures and dimensionalities illustrated in FIGS. 8A-8K are merely exemplary, and any suitable architectures and dimensionalities may be used to implement the techniques. For example, the architectures may include additional or alternative layers and may have different dimensionalities.

FIG. 8A provides an overview of the technique. As shown in FIG. 8A, a tissue image 801 is first divided into a plurality of image portions 802 by tiling the tissue image at one or more magnification levels. Each image portion 804 is encoded using a trained encoder 805 to obtain a representation 806-N of the image portion. The resulting variable-length set of representations 806-1 through 806-N is processed by a trained adapter 807 to obtain an adapted representation 808 of the tissue image. The adapted representation 808 is used to condition a denoising diffusion model 810, which iteratively denoises a noise input 809 via cross-attention layers 810-1 to generate a representation 811 of the RNA expression data. The representation 811 is then processed by a decoder 812 to generate the output 813, which may include decoded RNA expression data or values derived therefrom.

FIG. 8B illustrates an example architecture of the trained encoder 805 used to encode tissue image portions. As shown in FIG. 8B, the encoder takes an image portion 804 as input and processes it through an initial 2D convolution layer 815 to produce a feature map, which is then processed through a transformer stack 816 comprising transformer blocks 817. Each transformer block 817-1 comprises a sequence of normalization 817-1, self-attention 817-2, scale 817-3, normalization 817-4, MLP 817-5, and scale 817-6 layers. The output of the transformer stack is processed through a final normalization layer 818 and an output head 819 to produce a representation 806-N of the image portion. This architecture corresponds to a vision transformer trained using a self-supervised learning method, as described herein with respect to FIG. 1C.

FIG. 8C illustrates an example architecture of the trained adapter 807. As shown in FIG. 8C, the adapter takes as input the variable-length set of tile-level representations 806-1 through 806-N produced by the encoder. The representations are processed through two parallel branches: a first branch 820 comprising a linear layer 820-1, a Tanh activation 820-2, and a regularization layer 820-3; and a second branch 821 comprising a linear layer 821-1, a Sigmoid activation 821-2, and a regularization layer 821-3. The outputs of the two branches are combined by element-wise multiplication at a gate 822, passed through a linear attention scoring layer 823, normalized using a normalization layer 824, and aggregated at an aggregation layer 825 to produce a adapted representation 808 of the tissue image. This gated attention mechanism performs multiple instance learning to aggregate the variable-length bag of tile-level representations into a single fixed-size slide-level representation, as described herein with respect to FIG. 1C.

FIG. 8D illustrates an example architecture of the denoising diffusion model 810. As shown in FIG. 8D, the denoising diffusion model has a UNet architecture that takes an input feature representation 826 and the adapted representation 808 of the tissue image as inputs, and generates a representation 811 of the RNA expression data as output. The UNet comprises an initial 2D convolution layer 827, three downsampling blocks 828, 829, and 830, a mid block 831, and three upsampling blocks 832, 833, and 834, followed by a normalization layer 835, a final 2D convolution layer 836. A time embedding 838 is provided to the residual blocks throughout the network to encode the current diffusion timestep. Skip connections pass the outputs of corresponding downsampling blocks to their corresponding upsampling blocks. The adapted representation 808 of the tissue image is provided as a conditioning input to the cross-attention layers of the downsampling, mid, and upsampling blocks.

FIGS. 8E and 8F illustrate the internal architectures of the downsampling blocks of the denoising diffusion model. As shown in FIG. 8E, downsampling blocks 828 and 829 each comprise repeated sequences of residual block 840-1, cross-attention 840-2, residual block 840-3, and cross-attention 840-4 layers, followed by a downsampling operation 840-5. The adapted representation 808 of the tissue image and the time embedding 838 are provided as conditioning inputs to the cross-attention and residual block layers respectively. As shown in FIG. 8F, downsampling block 830 comprises residual blocks 830-1 and 830-2 without cross-attention layers, followed by an output 843, and receives the time embedding 838 as a conditioning input.

FIG. 8G illustrates the internal architecture of the mid block 831. As shown in FIG. 8G, the mid block comprises a residual block 845-1, a cross-attention layer 845-2, and a second residual block 845-3, receiving the adapted representation 808 of the tissue image and the time embedding 838 as conditioning inputs, and producing an output 846.

FIGS. 8H, 8I, and 8J illustrate the internal architectures of the three upsampling blocks of the denoising diffusion model. As shown in FIG. 8H, upsampling block 832 comprises a concatenation with skip state 848, followed by residual blocks 832-2, 832-3, and 832-4, and an upsampling operation 832-5, receiving the time embedding 838 as a conditioning input but no cross-attention conditioning. As shown in FIG. 8I, upsampling block 833 comprises a concatenation with skip state 852, followed by repeated sequences of residual block, cross-attention, residual block, and cross-attention layers, followed by an upsampling operation 833-7, receiving both the adapted representation 808 and the time embedding 838 as conditioning inputs. As shown in FIG. 8J, upsampling block 834 comprises a concatenation with skip state 855, followed by repeated sequences of residual block and cross-attention layers without an upsampling operation, receiving both the adapted representation 808 and the time embedding 838 as conditioning inputs.

FIG. 8K illustrates the architecture of the time embedding network used to generate the time embedding 838. As shown in FIG. 8K, the time embedding network takes a scalar timestep t as input, processes it through a sinusoidal positional encoding layer 856 to produce a sinusoidal embedding, which is then processed through a linear layer 857, an activation layer 858, and a second linear layer 858 to produce a time embedding 838 that is provided as a conditioning input to the residual blocks throughout the denoising diffusion model.

As an example, one or more models having the architectures described with respect to FIGS. 8A-8K were trained to generate representations of RNA expression data. The representations of RNA expression data were decoded to obtain RNA expression data including RNA expression level values for genes.

In the example, the encoder is a ViT-L, having the architecture shown in FIG. 8B, trained using the DINOv2 self-supervised teacher-student framework. Training data included H&E whole-slide images from which tissue-containing tiles of 224×224 pixels at 20× magnification were extracted. At inference, the encoder was applied to each tile to produce a 384-dimensional embedding per tile. The encoder weights were held fixed during all subsequent training stages.

The gated attention pooler, having the architecture shown in FIG. 8C, was trained to aggregate a variable-length bag of per-tile embeddings into a single 384-dimensional slide-level conditioning vector. During training, a fixed sequence length of 400 tiles was sampled with replacement from each slide per training iteration, providing implicit data augmentation. The pooler was trained jointly with the denoising diffusion model (FIGS. 8D-8K) using an AdamW optimizer with a learning rate of 1e-4 and a cosine schedule with 500 warmup steps, a batch size of 2,048 with gradient accumulation of 2 for an effective batch size of 4,096, bf16 mixed precision, and for up to 15,000 epochs. At inference, the pooler was applied to the full set of tiles from each slide without subsampling, and the resulting conditioning vector is pre-computed and cached for diffusion inference.

The denoising diffusion model, having the architecture shown in FIGS. 8D-8K, was trained to generate RNA expression data from tissue image data by learning to denoise LIAE latent representations conditioned on the slide-level conditioning vector produced by the gated attention pooler. Training data included paired H&E whole-slide images and RNA expression data. Gaussian noise was progressively added to the LIAE latent representations over 1,000 timesteps according to a squaredcos_cap_v2 beta noise schedule. The denoising diffusion model was trained to predict the added noise by minimizing an MSE loss. The conditioning input was randomly dropped with a probability of 15% during training to enable classifier-free guidance. The model was trained using the same optimizer, learning rate, schedule, batch size, and precision settings as the gated attention pooler.

At inference, a random noise tensor was iteratively denoised over 200 steps conditioned on the slide's conditioning vector, repeated three times with different random seeds and averaged to produce a stabilized latent representation, which was rescaled and decoded through the frozen LIAE decoder to produce a reconstructed TPM gene expression vector. For uncertainty estimation, sampling may be repeatedK≥15 times.

Gene-wise performance was measured by Pearson correlation (mean 0.67), Spearman correlation (mean 0.64), and mean absolute percentage error (MAPE 18 percent) versus matched RNA-seq. Model-to-ground-truth correlations were compared against a zero-information baseline of 0.02. These results indicate that the techniques described by the inventors can be used to accurately generate RNA expression data for a subject from a tissue image.

FIG. 9 shows an example tissue image from a subject and signature scores computed from RNA expression data according to the above-described techniques. Spearman correlation of Ground Truth and reconstructed signature scores achieves 0.93, which indicates that the diffusion model has captured biology and can be used to generate useful expression data if absent.

Generating Gene Group Signature Scores from Tissue Image Data

Aspects of the technology described herein relate to generating output values, such as signature scores for one or more gene groups, directly from tissue image data for a subject. Illustrative techniques for generating output values from tissue image data are described herein including at least with respect to FIG. 1D and FIG. 3D. FIGS. 10A-11D provide additional details and examples relating to these techniques.

FIGS. 10A-10D illustrate an example technique for generating signature scores for gene groups directly from a tissue image, and example architectures of the components used to implement that technique, according to some embodiments of the technology described herein. It should be appreciated that the specific architectures and dimensionalities illustrated in FIGS. 10A-10D are merely exemplary, and any suitable architectures and dimensionalities may be used to implement the techniques. For example, the architectures may include additional or alternative layers and may have different dimensionalities.

FIG. 10A provides an overview of the technique. As shown in FIG. 10A, a tissue image 1001 is first divided into a plurality of image portions 1002 by tiling the tissue image. Each image portion 1004 is encoded using a trained encoder 1005 to obtain a respective representation 1006 of the image portion. The resulting variable-length set of representations 1007, comprising representations of all encoded image portions, is processed by a trained adapter 1008 to obtain an adapted representation 1009 of the tissue image as a whole. The adapted representation 1009 is then provided as input to one or more regression heads 1010, each comprising a linear layer 1010-1, to generate one or more signature scores 1011 for the subject. As shown in FIG. 10A, the trained encoder 1005 takes image portions as input and produces representations, and the trained adapter 1008 maps the variable-length set of tile-level representations to a single adapted representation of the tissue image. In some embodiments, the trained encoder 1005 is a pre-trained foundation model used as a fixed feature extractor, as described herein with respect to FIG. 1D.

FIG. 10B illustrates an example architecture of the trained adapter 1008. As shown in FIG. 10B, the trained adapter takes as input the variable-length set of tile-level representations 1007 produced by the trained encoder. The representations are first processed by a feature extraction network 1012 to produce feature representations 1013 of reduced dimensionality. The feature representations 1013 are then processed by a gated attention network 1014, which computes a set of attention weights 1015 reflecting the relative importance of each image portion for the downstream prediction task. The feature representations 1013 and attention weights 1015 are combined by an attention-weighted aggregation operation 1016 to produce a single adapted representation 1017 of the tissue image as a whole. This adapted representation 1017 is then provided as input to the regression head(s) 1010 to generate the signature scores 1011.

FIG. 10C illustrates an example architecture of the feature extraction network 1012. As shown in FIG. 10C, the feature extraction network takes as input the tile-level representations 1007 produced by the trained encoder and processes each representation independently and identically through a sequence of operations comprising a linear projection layer 1012-1, a non-linear activation layer 1012-2, and a regularization layer 1012-3, to produce a corresponding feature representation 1013 of reduced dimensionality for each tile. The feature extraction network is applied independently to each tile-level representation, such that the same learned weights are used to process every tile regardless of the total number of tiles in the slide.

FIG. 10D illustrates an example architecture of the gated attention network 1014. As shown in FIG. 10D, the gated attention network takes as input the feature representations 1013 produced by the feature extraction network and processes them through two parallel branches: a first branch 1018 comprising a linear projection layer 1018-1, a Tanh activation 1018-2, and a regularization layer 1018-3; and a second branch 1019 comprising a linear projection layer 1019-1, a Tanh activation 1019-2, and a regularization layer 1019-3. The outputs of the two branches are combined by an attention scoring operation 1020, which computes a scalar attention logit for each tile by performing element-wise multiplication of the two branch outputs and passing the result through a linear layer 1021. The scalar attention logits are normalized across all T tiles by a normalization layer 1022, such as a softmax operation, to produce a probability distribution over tiles that sums to one, yielding the attention weights 1015. The attention weights 1015 reflect the learned importance of each image portion for the downstream prediction task, with higher weights assigned to image portions that are more morphologically informative.

As an example, one or more models having the architectures described with respect to FIGS. 10A-10D were trained to generate gene group signature scores.

A feature extraction network (FIG. 10C) and gated attention network (FIG. 10D) were trained end-to-end jointly with the regression head(s). Training data included paired H&E whole-slide images and RNA expression data, from which 55 continuous gene group signature scores were derived using a gene set scoring method applied to matched RNA sequencing data. The gene groups are listed in Table 2. Tile embeddings were pre-computed using a frozen pre-trained histopathology foundation model, H-Optimus-0, and stored in a Zarr archive together with the corresponding signature scores. Slides with no matched signature data were excluded from all training and validation batches. Each included training sample was assigned a scalar sample weight used during loss computation, which was used to up-weight samples with rare or extreme signature profiles.

At each training iteration, a batch of slides was sampled and a fixed number of tiles is drawn with replacement from the full tile pool of each slide, yielding a batch tensor of fixed shape regardless of the actual number of tiles per slide. This sampling with replacement strategy ensured a fixed, uniform sequence length across all slides within a batch and provided implicit data augmentation by exposing the model to different tile subsets from the same slide across training epochs. The default batch size was 50 slides per batch with 400 tiles sampled per slide. The model was trained by minimizing a sample-weighted MSE loss computed jointly over all 55 signatures:

$${L}_{MSE} = \frac{1}{B} * {\sum }_{b = 1}^{B} {w}_{b} * [ \frac{1}{55} * {\sum }_{k} {( {\hat{y}}_{b , k} - {y}_{b , k} )}^{2} ]$$

where ŷ_{b,k} is the predicted score for slide b and signature k, ŷ_{b,k} is the corresponding ground truth score, and w_{b} is the sample weight for slide b. The model was optimized using an Adam W optimizer with a learning rate of 1×10⁻⁴ and a weight decay of 0.01. The feature network hidden dimensionality H was 256, the attention hidden dimensionality G was 256, and the dropout rate p was 0.2. The model was trained for up to 1,000 epochs, with the best model checkpoint selected based on the lowest validation loss observed across all epochs. Training may be terminated early if the validation loss fails to improve for 10 consecutive epochs. The following metrics were logged at the end of each epoch to enable monitoring and reproducibility: training loss, validation loss, per-signature MSE and MAE across all 55 signatures, and the current learning rate.

At inference, tile embeddings for a given slide were loaded from the Zarr storage archive and passed in full (e.g., without tile sampling) through the trained feature network, gated attention network, and regression head(s) to produce the inference outputs. For each slide, the model produced three outputs: a vector of 55 predicted signature scores, one per gene group; a bag embedding comprising the attention-weighted aggregated slide representation, suitable for use as a downstream feature vector for secondary analyses; and a set of per-tile attention weights reflecting the contribution of each tile to the slide-level prediction. In the single-head variant, the attention weights have shape T×1, producing a single spatial attention map for the slide. In the multi-head variant, the attention weights have shape 55×T, producing a separate spatial attention map for each of the 55 predicted signatures. Inference results are serialized in AnnData Zarr format, comprising slide-level metadata, predicted signature scores, attention scores, and signature identifiers. The per-tile attention weights may be projected back onto the original tissue image to generate spatial attention heatmaps identifying the morphological regions most informative for each predicted signature score, as shown, for example, in FIGS. 11A-11C.

### RNA Expression Data

As used herein, RNA expression data refers to data that quantifies the abundance of RNA transcripts in a biological sample, reflecting the degree to which individual genes are transcribed into RNA at the time the sample was obtained. RNA expression data may be acquired using any method known in the art including, but not limited to, whole transcriptome sequencing, whole exome sequencing, total RNA sequencing, mRNA sequencing, targeted RNA sequencing, RNA exome capture sequencing, next generation sequencing, deep RNA sequencing, spatial transcriptomics, and/or microarray assays.

In some embodiments, RNA expression data is derived from sequencing data obtained from a biological sample using any suitable sequencing technique and/or apparatus. In some embodiments, the sequencing apparatus may be selected from any suitable sequencing apparatus known in the art including, but not limited to, Illumina, SOLid, Ion Torrent, PacBio, a nanopore-based sequencing apparatus, a Sanger sequencing apparatus, or a 454 sequencing apparatus. In some embodiments, the sequencing apparatus is an Illumina sequencing apparatus, such as a NovaSeq, NextSeq, HiSeq, MiSeq, or MiniSeq apparatus. In some embodiments, the sequencing data may be obtained in a text-based file format, such as FASTQ, FASTA, BAM, or SAM format. In some embodiments, the sequencing data and/or RNA expression data may be accessed from at least one computer storage medium on which the sequencing data and/or RNA expression data is stored, or may be received from one or more sources via a communication network, such as from a server.

In some embodiments, the sequencing data is processed to produce RNA expression data using one or more bioinformatics methods or software tools, such as RNA sequence quantification tools and genome annotation tools. In some embodiments, microarray expression data is processed using a bioinformatics software package to produce expression data. In some embodiments, the expression data is acquired through bulk RNA sequencing, which may include obtaining expression levels for each gene across RNA extracted from a population of input cells comprising a mixture of different cell types. In some embodiments, bulk sequencing data comprises at least 1 million reads, at least 5 million reads, at least 10 million reads, at least 20 million reads, at least 50 million reads, or at least 100 million reads. In some embodiments, the expression data is acquired through single-cell RNA sequencing, which may include sequencing individual cells to obtain cell-level expression measurements. In some embodiments, RNA expression data is derived from spatial transcriptomics platforms, in which expression levels are measured at spatially resolved locations within a tissue section.

RNA expression data may be represented as raw read counts, or as normalized abundance values. In some embodiments, processing the sequencing data to obtain RNA expression data includes normalizing the sequencing data to transcripts per kilobase million (TPM) units. In some embodiments, the RNA expression levels may instead be converted to another type of unit, such as reads per kilobase million (RPKM), fragments per kilobase million (FPKM), or counts per million (CPM). In some embodiments, the RNA expression levels may be log-transformed following normalization. In some embodiments, the log transformation may be omitted, or one or more other transformations may be applied.

Expression data for a plurality of genes may be used for any of the methods described herein. In some embodiments, expression levels may be obtained for at least 25 genes, at least 50 genes, at least 100 genes, at least 500 genes, at least 1,000 genes, at least 5,000 genes, at least 10,000 genes, at least 15,000 genes, or at least 20,000 genes. In some embodiments, expression levels may be obtained for at most 25 genes, at most 100 genes, at most 1,000 genes, at most 5,000 genes, at most 10,000 genes, at most 15,000 genes, or at most 20,000 genes. It should be appreciated that any of the above-listed upper bounds may be coupled with any of the above-listed lower bounds. In some embodiments, the number of genes for which expression levels are obtained includes all of the genes of the subject. It should be appreciated that RNA expression data is not limited to any particular RNA expression measurement platform, sequencing technology, normalization approach, or data representation, as aspects of the technology described herein are not limited in this respect.

### Signature Scores

Aspects of the technology described herein relate to generating, predicting, or deriving signature scores for one or more gene groups for a subject. As described herein, signature scores for a subject may be generated from a tissue image using the trained multimodal generative model described herein with respect to FIGS. 1D and 10A-11D, or may be derived from RNA expression data. In some embodiments, signature scores are provided as output values of the trained multimodal generative model. In some embodiments, signature scores are used as ground truth training targets for training the trained multimodal generative model. In some embodiments, signature scores are used as inputs to one or more downstream analyses, such as tumor microenvironment characterization, treatment response prediction, patient stratification, or clinical trial cohort identification, as described herein.

A signature score for a gene group may refer to a numeric value that summarizes the overall activity or enrichment of a set of genes within a given biological sample. Signature scores may be used to characterize biological processes, cellular states, or pathway activities that are relevant to disease biology and clinical outcomes, including for example immune cell infiltration, stromal activation, angiogenesis, cellular proliferation, and other tumor microenvironment processes. Examples of gene groups for which signature scores may be generated are listed in Table 2. In some embodiments, a respective signature score is determined or generated for one or more (e.g., one, at least 2, at least 5, at least 10, at least 20, at least 25, at least 30, at least 40, at least 55, etc.) of the gene groups. In some embodiments, a signature score for a gene group may be determined from at least some (e.g., at least 2, at least 3, at least 5, at least 10, etc.) genes in the gene group.

TABLE 2
Gene groups.

| Gene Signature | Gene List |
|---|---|
| Main4 Proliferation rate | AURKB CCNE1 MKI67 CCNB1 CCND1 E2F1 CD |
| | K2 ESCO2 PLK1 AURKA BUB1 CETN3 MCM2 |
| | MYBL2 MCM6 |
| Main4 Apoptosis up | FAS PMAIP1 APAF1 CASP3 TNFRSF1OB CASP7 |
| | CASP8 XAF1 TNFRSF10A BIK CASP8AP2 CASP6 |
| | CYLD TNFRSF12A BCL10 CASP2 IER3 BCL2L11 |
| Main4 Metastasis signature | SERPINH1 CDH2 PARP1 HGF MMP9 MET HPSE |
| SOLID | |
| Main4 Cell senescence | GDF15 IGFBP7 IGFBP2 MMP3 TGFB1 BCL2L1 C |
| | DKN2A IGFBP3 PLAU CDKN2B GLB1 CDKN1A |
| | IL6 BCL2L2 IL1B CXCL1 TNFRSF10D ATF3 DPP |
| | 4 CCL2 CXCL8 CEBPB SERPINE1 STAT1 ITGB3 |
| Main4 Glycolysis SOLID | BPGM ENO1 LDHA PGK1 GPI TPI1 GPD2 PFK |
| | P SLC16A3 ALDOA |
| Main4 EMT signature | RUNX2 FOXM1 TWIST1 SNAI1 SNAI2 |
| Main4 MHCI | TAP2 HLA-A TAP1 NLRC5 HLA-C B2M HLA- |
| | B TAPBP |
| Main4 MHCII | HLA-DMB HLA-DMA HLA-DRA HLA-DPA1 HLA- |
| | DRB1 HLA-DQA1 HLA-DPB1 CIITA HLA-DQB1 |
| Main4 Coactivation receptors | CD28 TNFRSF9 CD40 ICOS CD27 TNFRSF4 |
| Main4 Coactivation ligands | CD80 CD70 ICOSLG CD83 TNFSF4 TNFSF9 CD8 |
| | 6 CD40LG |
| Main4 Effector cells | ZAP70 GZMB PRF1 EOMES GNLY IFNG FASLG |
| | TBX21 GZMA CD8A GZMH |
| Main4 CD4 T cells | TRAC CD28 TRAT1 CD4 CD40LG |
| Main4 B cells | CR2 TNFRSF13C BLK CD79A MS4A1 PAX5 TNF |
| | RSF13B FCRL5 CD79B STAP1 CD22 TNFRSF17 |
| | CD19 |
| Main4 TLS SOLID | CCL21 CCR7 BCL6 SELL LAMP3 CXCR4 CXCL |
| | 13 CCL19 CD86 |
| Main4 Antitumor cytokines | TNFSF10 CCL3 TNF IL21 IFNB1 IFNG |
| Main4 Lymphoid checkpoints | CTLA4 KLRC1 PVRIG BTLA LAG3 TIGIT PDCD1 |
| Main4 Myeloid checkpoints | C10orf54 CD274 HAVCR2 CD276 PDCD1LG2 |
| Main4 Treg | IKZF2 CCR8 CTLA4 FOXP3 TNFRSF18 IL2RA I |
| | KZF4 |
| Main4 Neutrophil signature | VNN2 CXCR2 FPR2 CSF3R SIGLEC5 CXCR1 FC |
| | GR3B ADGRG3 |
| Main4 M2 signature | MSR1 CSF1R FPR3 CD163 CD68 MS4A4A MRC1 |
| | CD209 |
| Main4 SPP1 Macrophages | CCL7 GAL3ST4 CXCL5 SPP1 APOC2 CLEC5A |
| Main4 Protumor cytokines | IL22 TGFB2 IL10 TGFB1 IL6 MIF TGFB3 |
| Main4 Metastasis promotion | CXCL8 TGFB1 TNFSF11 OSM CCR6 CCR4 CXC |
| SOLID | R4 IL1B HIF1A IL15RA HGF |
| Main4 CAF associated | MMP2 COL6A3 MFAP5 LUM COL1A1 CD248 FA |
| | P COL1A2 ACTA2 THY1 PDGFRB COL5A1 PDG |
| | FRA |
| Main4 iCAF | IGF1 CXCL14 CFD CXCL12 IL6 DCN CTSK PD |
| | GFRA DPT LUM CCL2 AGTR1 NR2F1 PTGDS |
| | FGF7 FIGF IGF2 THY1 PDGFRB APOD |
| Main4 Matrix | FN1 LAMB3 COL11A1 COL1A1 LAMA3 COL1A2 |
| | LAMC2 TNC VCAN COL3A1 LGALS3 LGALS9 |
| | COL4A1 ELN COL5A1 |
| Main4 Matrix remodeling | MMP2 ADAMTS4 MMP3 MMP7 CA9 MMP9 LO |
| | X PLOD2 MMP11 MMP12 MMP1 ADAMTS5 |
| Main4 PVL | MYH11 NOTCH3 PPP1R14A RGS5 ACTA2 MCAM |
| | TAGLN PDGFRB THY1 SPARCL1 MYL9 |
| Main4 Angiogenesis | PGF VEGFA VEGFC VWF VEGFB PDGFC CDH5 |
| | ANGPT2 TEK FLT1 ANGPT1 KDR |
| Main4 Endothelium | VWF ECSCR MMRN1 CLEC14A MMRN2 NOS3 |
| | CDH5 ENG FLT1 ROBO4 KDR |
| Main4 Hypoxia factors | HK2 FUT11 LDHA P4HA1 PGK1 CA9 SLC2A1 |
| | LOX TPI1 NDRG1 PDK1 CA12 PFKFB3 EPAS1 |
| | ALDOA |
| Main4 Type 1 inflammation | FLT3LG IL27 CCL5 TNF TNFSF11 CD70 IFNG I |
| SOLID | L18 TNFSF9 CD40LG IL15RA |
| Main4 M1 polarization SOLID | CCL21 IL23A CSF2 IL27 IL12B TNF IFNG |
| Main4 Tumor growth inhibition | CXCR2 IL1RN LTA ACKR2 IL1R2 ACKR1 IFNG |
| SOLID | |
| Main4 Invasion and metastasis | TIMP3 TIMP2 ACKR2 CCL28 ACKR1 |
| inhibition SOLID | |
| Main4 Angiogenesis inhibition | SERPINF1 TNFSF15 ARRB2 IL12A VASH1 IFNG |
| SOLID | MMRN2 CXCR3 IL12B ACKR1 |
| Main4 B cell activation SOLID | TNFSF13 IL10 TNFSF13B IL4 IFNG |
| Main4 T B cell recruitment | CCL21 CCL17 CXCL9 CXCL10 CCL5 CCL20 CX |
| SOLID | CL11 CCL22 CXCL13 CCL19 |
| Main4 Mph DC recruitment | CCL17 CCL2 TGFB1 CCL18 CSF1 CXCL12 CCL3 |
| SOLID | XCL1 |
| Main4 Pro-inflammatory | CXCL1 CCL2 LIF TNF IL23A IL1A CXCL2 IL1 |
| cytokines SOLID | B CCL4 IL6 CCL3 |
| Main4 Nph recruitment | CXCL1 CSF2 CXCL3 CXCL5 CXCL8 PPBP CXC |
| activation SOLID | L6 PF4 CXCL2 CSF3 |
| Main4 Th2 response SOLID | CCL13 CCL8 IL13 TSLP IL10 IL11 IL4 |
| Main4 M2 polarization SOLID | TGFB3 TGFB2 IL10 CCL24 IL33 CCL18 |
| Main4 Eosinophils recruitment | CCL26 CCL13 CCL8 CCL11 CCL24 |
| SOLID | |
| Main4 Eph Bas activation | IL13 IL1RL1 ENPP3 IL5RA ANGPT1 IL33 |
| SOLID | |
| Main4 Suppressive factors | TGFB2 TGFBI TGFB1 IL10 EBI3 SOCS1 TDO2 |
| SOLID | SOCS3 IL18BP IDO1 FGL2 TGFB3 |
| Main4 Stromal Suppressive | TGFB2 TSLP TGFBI IL6 IL11 TDO2 TGFB3 |
| factors SOLID | |
| Main4 Exclusion SOLID | VEGFA FGF2 TNFAIP6 TGFBI PDGFC CXCL12 |
| | AXL GAS6 EDNRB |
| Main4 Treg polarization SOLID | CCL17 TGFB2 IL10 TGFB1 IL2RA CCL22 IDO1 |
| | TGFB3 |
| Main4 Tumor growth stimulation | FGF7 IGF1 FGF10 EGF IL6 TGFB3 HGF |
| SOLID | |
| Main4 Induction of EMT SOLID | CCL7 CCL8 CXCL8 LIF IL6 TNFSF11 ARTN |
| Main4 Angiogenesis induction | VEGFA VEGFC PDGFB TGFB1 PDGFC TNFSF12 |
| SOLID | FGF2 PDGFA |
| Main4 CAF recruitment SOLID | MMP2 FGF1 TGFB2 TGFB3 TGFB1 CXCL12 IL6 |
| | FGF2 |
| IFN response | PARP12 PLSCR1 IFI6 DHX58 IFI44 STAT2 MX1 |
| | IFITM1 CXCL10 DDX58 IFI27 IFITM2 LAP3 IFIT |
| | 1 OASL IFI44L OAS3 OAS1 IRF7 LGALS3BP L |
| | Y6E UBE2L6 OAS2 TRIM22 RTP4 IFIT2 RSAD2 |
| | ISG15 NMI IFIH1 |
| MDSC signature | S100A12 EREG SERPINA1 CD300E CD55 S100A9 |
| | FCN1 LYZ VCAN S100A8 SLC11A1 CSF3R TIM |
| | P1 |

In some embodiments, a signature score is generated using a gene set enrichment analysis (GSEA) technique, using RNA expression levels of at least some genes in a set of genes. In some embodiments, using a GSEA technique comprises using single-sample GSEA. Aspects of single sample GSEA (ssGSEA) are described in Barbie et al. Nature. 2009 Nov. 5; 462 (7269): 108-112, the entire contents of which are incorporated by reference herein. In some embodiments, ssGSEA is performed according to the following formula:

$$ssGSEA score = \frac{{\sum }_{i}^{N} {r}_{1}^{1.25}}{{\sum }_{i}^{N} {r}_{1}^{0.25}} - \frac{( M - N + 1 )}{2}$$

where ri represents the rank of the ith gene in expression matrix, where N represents the number of genes in the gene set, and where M represents total number of genes in expression matrix. Additional, suitable techniques of performing GSEA are known in the art and are contemplated for use in the methods described herein without limitation. In some embodiments, an enrichment score is calculated by performing ssGSEA on expression data from a plurality of subjects, for example expression data from one or more cohorts of subjects, such as TCGA, Metabric, FUSCCTNBC, GSE103091, GSE106977, GSE21653, GSE25066, GSE41998, GSE47994, GSE81538, GSE96058, etc., in order to produce a plurality of enrichment scores.

### Subjects

Aspects of this disclosure relate to data that has been obtained for one or more subjects. In some embodiments, a subject is a mammal (e.g., a human, a mouse, a cat, a dog, a horse, a hamster, a cow, a pig, or other domesticated animal, a farm animal (e.g., livestock), a sport animal, a laboratory animal, a pet, and a primate). In some embodiments, a subject is a human. In some embodiments, a subject is an adult human (e.g., of 18 years of age or older). In some embodiments, a subject is a child (e.g., less than 18 years of age).

### Biological Samples

Aspects of the disclosure relate to techniques for generating data of a target modality for a subject from input data of one or more other modalities. In some embodiments, the prediction is generated based on data obtained from one or more biological samples that have been obtained from a subject.

The biological sample may be from any source in the subject's body including, but not limited to, any fluid such as blood (e.g., whole blood, blood serum, or blood plasma), lymph node, breast, etc. Other source in the subject's body may be from saliva, tears, synovial fluid, cerebrospinal fluid, pleural fluid, pericardial fluid, ascitic fluid, and/or urine, hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, bone, bone marrow, brain, thymus, spleen, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, one or more pieces of tissue(s) or organ(s). In some embodiments, the biological sample comprises breast tissue sample of the subject. In some embodiments, a breast tissue sample comprises one or more cell types derived from a breast (e.g., epithelial cells, secretory luminal cells, basal/myoepithelial cells, etc.). In some embodiments, a breast tissue sample comprises tumor cells.

In some embodiments, a tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 February;21 (2): 253-5), and Biological sample collection, processing, storage and information management by Vaught and Henderson (IARC Sci Publ. 2011; (163): 23-42). Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 40C for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris Cl; 0.5 mM EDTA, pH 9.0)) and other coagulants, and Acids Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25 °C). In some embodiments, the sample is stored under refrigeration (e.g., 4 °C). In some embodiments, the sample is stored under freezing conditions (e.g., −20 °C). In some embodiments, the sample is stored under ultralow temperature conditions (e.g., −50 °C to −80 °C). In some embodiments, the sample is stored under liquid nitrogen (e.g., −170 °C). In some embodiments, a biological sample is stored at −60 °C to −8 °C (e.g., −700 °C) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Methods of Treatment

Aspects of the disclosure relate to techniques for identifying a treatment for a subject based on data generated using the techniques described herein, and optionally administering the identified treatment to the subject. In some embodiments, the generated data of the target modality and/or the input data may be used to identify a therapeutic agent that is likely to be effective for the subject, for example by characterizing the subject's tumor microenvironment, identifying molecular subtypes, or predicting response to a particular therapeutic agent, as described herein. In some embodiments, the techniques described herein include recommending administration of a therapeutic agent to a subject and/or administering a therapeutic agent to the subject based at least in part on the generated data.

In some embodiments, a therapeutic agent (e.g., an anti-cancer therapeutic agent) is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), trastuzumab deruxtecan (Enhertu), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor (e.g., nivolumab (Opdivo), pembrolizumab (Keytruda), atezolizumab (Tecentriq), avelumab (Bavencio), durvalumab (Imfinzi)), a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

In some embodiments, methods described by the disclosure further comprise a step of administering one or more therapeutic agents to the subject. In some embodiments, a subject is administered one or more (e.g., 1, 2, 3, 4, 5, or more) therapeutic agents.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., leukocyte immunoprofile type, tumor microenvironment, tumor formation, tumor growth, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one to four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced doses, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward cancer.

Alleviating cancer includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

### Computer Implementation

An illustrative implementation of a computer system 1200 that may be used in connection with any of the embodiments of the technology described herein (e.g., such as the processes of FIGS. 3A-3E) is shown in FIG. 12. The computer system 1200 includes one or more processors 1210 and one or more articles of manufacture that comprise non-transitory computer-readable storage media (e.g., memory 1220 and one or more non-volatile storage media 1230). The processor 1210 may control writing data to and reading data from the memory 1220 and the non-volatile storage media 1230 in any suitable manner, as the aspects of the technology described herein are not limited to any particular techniques for writing or reading data. To perform any of the functionality described herein, the processor 1210 may execute one or more processor-executable instructions stored in one or more non-transitory computer-readable storage media (e.g., the memory 1220), which may serve as non-transitory computer-readable storage media storing processor-executable instructions for execution by the processor 1210.

Computing system 1200 may include a network input/output (I/O) interface 1240 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 1200 may also include one or more user I/O interfaces 1250, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device. The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### Example Aspects

Some embodiments provide for a method for generating data of a target modality for a subject, the method comprising: using at least one processor to perform: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

Some embodiments provide for a system, comprising: at least one processor; and at least one non-transitory computer-readable storage medium storing processor-executable instructions that, when executed by the at least one processor, cause the at least one processor to perform a method for generating data of a target modality for a subject, the method comprising: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

Some embodiments provide for at least one non-transitory computer-readable storage medium storing processor-executable instructions that, when executed by at least one processor, cause the at least one processor to perform a method for generating data of a target modality for a subject, the method comprising: obtaining input data of one or more modalities, the input data having been previously obtained for the subject, the one or more modalities excluding the target modality; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and generating the data of the target modality by processing, using the trained multimodal generative model, the one or more adapted representations of the input data.

In some embodiments, the one or more modalities and the target modality are selected from: genomic data, epigenomic data, transcriptomic data, image data, cytometry data, spatial-omics data, microbiome data, proteomic data, metabolomic data, cell count data, single-cell omics data, liquid biopsy data, immune cell repertoire data, and clinical data.

Some embodiments further comprise identifying a treatment for a subject based on the input data and/or the generated data.

Some embodiments further comprise administering the identified treatment to the subject. Some embodiments further comprise identifying the subject as a member of a clinical trial cohort based on the input data and/or the generated data.

Some embodiments further comprise: prior to generating the data of the target modality, modifying the input data, thereby modeling an altered biological state of the subject.

In some embodiments, the at least one encoder comprises a respective modality-specific encoder for each of the one or more modalities.

In some embodiments, the input data comprises first input data of a first modality and second input data of a second modality, and wherein encoding the input data using the at least one encoder comprises: encoding the first input data using a first modality-specific encoder to obtain a first representation of the first input data; and encoding the second input data using a second modality-specific encoder to obtain a second representation of the second input data, wherein the second modality-specific encoder is different from the first modality-specific encoder.

In some embodiments, the at least one trained adapter comprises a respective modality-specific trained adapter for each of the one or more modalities.

In some embodiments, the at least one trained adapter is trained to map the one or more representations of the input data to a target representation space to obtain the one or more adapted representations of the input data, and wherein the one or more adapted representations share a common dimensionality in the target representation space.

In some embodiments, the at least one trained adapter comprises one or more linear projection layers configured to project the one or more representations of the input data into the one or more adapted representations of the input data.

Some embodiments further comprise: combining the one or more adapted representations of the input data to obtain a combined representation of the input data, wherein the combining comprises at least one of: concatenating, summing, averaging, or using a learned aggregation function, wherein processing the one or more adapted representations of the input data using the trained multimodal generative model comprises processing the combined representation of the input data using the trained multimodal generative model.

In some embodiments, processing the one or more representations of the input data using the trained multimodal generative model comprises processing each of the one or more adapted representations using a respective set of one or more cross-attention layers.

In some embodiments, each of the one or more modalities has a respective trained encoder and a respective trained adapter.

In some embodiments, the trained multimodal generative model is a trained denoising model.

In some embodiments, the trained denoising model is a trained UNet-based diffusion model and/or a trained transformer-based diffusion model.

In some embodiments, generating the data of the target modality comprises iteratively denoising a noise input using the trained denoising model using a denoising diffusion implicit model (DDIM) sampling procedure.

In some embodiments, the trained denoising model is conditioned on the one or more adapted representations of the input data during a denoising process performed by the trained denoising model.

In some embodiments, generating the data of the target modality comprises iteratively denoising, using the trained denoising model, a noise input conditioned on the one or more adapted representations.

In some embodiments, the input data of the one or more modalities comprises RNA expression data and the data of the target modality comprises tissue image data, wherein encoding the input data using the at least one trained encoder comprises encoding the RNA expression data using the at least one trained encoder to obtain a representation of the RNA expression data, wherein processing the one or more representations of the input data using the at least one trained adapter comprises processing the representation of the RNA expression data using the at least one trained adapter to obtain an adapted representation of the RNA expression data, and wherein generating the data of the target modality by processing the one or more adapted representations of the input data using the trained multimodal generative model comprises generating the tissue image data by processing the adapted representation of the RNA expression data using the trained multimodal generative model.

In some embodiments, the at least one trained encoder comprises an autoencoder, wherein the at least one trained adapter comprises at least one linear projection layer and at least one non-linear activation layer, and wherein the trained multimodal generative model comprises a trained denoising model conditioned on the adapted representation of the RNA expression data.

In some embodiments, the input data of the one or more modalities comprises tissue image data and the data of the target modality comprises RNA expression data, the tissue image data comprising a tissue image comprising a plurality of image portions, wherein encoding the input data using the at least one trained encoder comprises encoding image portions of the plurality of image portions using the at least one trained encoder to obtain respective representations of the image portions, wherein processing the one or more representations of the input data using the at least one trained adapter comprises processing the representations of the image portions using the at least one trained adapter to obtain an adapted representation of the tissue image, and wherein generating the data of the target modality by processing the one or more adapted representations of the input data using the trained multimodal generative model comprises generating a representation of the RNA expression data by processing the adapted representation of the tissue image using the trained multimodal generative model.

In some embodiments, the at least one trained encoder comprises a vision transformer (ViT), wherein the at least one trained adapter comprises a gated attention network, and wherein the trained multimodal generative model comprises a trained denoising model conditioned on the adapted representation of the tissue image.

In some embodiments, the at least one trained encoder comprises a vision transformer (ViT), wherein the at least one trained adapter comprises a feature extraction network and a gated attention network, and wherein the trained multimodal generative model comprises a trained regression model comprising a linear output layer configured to output a plurality of values for the subject.

Some embodiments further comprise training the at least one encoder, the at least one adapter, and/or the multimodal generative model.

Some embodiments further comprise training the multimodal generative model, the training comprising: obtaining training data comprising (i) target training data of the target modality, and (ii) representations of input training data of the one or more modalities; processing one or more of the representations of the input training data using the at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

In some embodiments, the at least one trained encoder is a pre-trained encoder with weights that are fixed during the training of the multimodal generative model and/or the at least one adapter.

Some embodiments provide for a method for imputing missing modality data for a subject, the method comprising: using at least one processor to perform: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some embodiments provide for a system, comprising: at least one processor; and at least one when executed by the at least one processor, cause the at least one processor to perform a method for imputing missing modality data for a subject, the method comprising: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some embodiments provide for at least one non-transitory computer-readable storage medium storing processor-executable instructions that, when executed by at least one processor, cause the at least one processor to perform a method for imputing missing modality data for a subject, the method comprising: obtaining input data of one or more modalities, the input data having been previously obtained for the subject, wherein data of a target modality is unavailable for the subject; encoding the input data using at least one trained encoder to obtain one or more representations of the input data; processing the one or more representations of the input data using at least one trained adapter to obtain one or more adapted representations of the input data adapted for subsequent processing using a trained multimodal generative model; and imputing the data of the target modality by processing, using the trained multimodal generative model, the one or more adapted representations of the input data.

Some embodiments provide for a method for generating, from RNA expression data, a tissue image for a subject, the method comprising: using at least one processor to perform: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

Some embodiments provide for a system comprising: at least one processor; and at least one when executed by the at least one processor, cause the at least one processor to perform a method for generating, from RNA expression data, a tissue image for a subject, the method comprising: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

Some embodiments provide for at least one non-transitory computer-readable storage medium storing processor-executable instructions that, when executed by at least one processor, cause the at least one processor to perform a method for generating, from RNA expression data, a tissue image for a subject, the method comprising: obtaining RNA expression data derived from a biological sample previously obtained from a subject; encoding the RNA expression data using a trained encoder to obtain a representation of the RNA expression data; processing the representation of the RNA expression data using a trained adapter to obtain an adapted representation of the RNA expression data for subsequent processing using a trained multimodal generative model; and generating the tissue image for the subject by processing, using the trained multimodal generative model, the adapted representation of the RNA expression data.

In some embodiments, obtaining the RNA expression data comprises obtaining a plurality of expression level values for a respective plurality of genes.

In some embodiments, the plurality of genes comprises at least 5,000 genes, at least 10,000 genes, at least 15,000 genes, at least 20,000 genes, or any number of genes between 5,000 and 25,000.

Some embodiments further comprise modifying one or more of the expression level values prior to encoding the RNA expression data.

In some embodiments, the trained encoder is an autoencoder.

In some embodiments, the autoencoder is an encoder portion of a latent image autoencoder (LIAE) or an encoder portion of a vector quantized variational autoencoder (VQ-VAE).

In some embodiments, the autoencoder is the encoder portion of the LIAE, and wherein the encoder portion of the LIAE comprises: a projection block configured to project the RNA expression data to an intermediate representation; a reshaping operation configured to reshape the intermediate representation into a two-dimensional feature map; and a plurality of residual blocks configured to process the two-dimensional feature map.

In some embodiments, each residual block of the plurality of residual blocks comprises: a first convolutional layer, a first group normalization layer, a first non-linear activation layer, a second convolutional layer, a second group normalization layer, a skip connection, and a second non-linear activation layer.

In some embodiments, the projection block is configured to project the RNA expression data to the intermediate representation having a dimensionality of 1,024, and wherein the reshaping operation is configured to reshape the intermediate representation into a 32×32 two-dimensional feature map.

In some embodiments, the trained adapter is configured to map the representation of the RNA expression data to the adapted representation of the RNA expression data having a dimensionality matching a conditioner input of the trained multimodal generative model.

In some embodiments, the trained multimodal generative model comprises a trained denoising model having original cross-attention layers and supplementary cross-attention layers, and wherein the adapted representation of the RNA expression data comprises a sequence of multiple tokens, each of the multiple tokens having an embedding dimension of 768, wherein a first portion of the sequence is provided as a key-value input to the supplementary cross-attention layers of the trained denoising model and a second portion of the sequence is provided as a key-value input to the original cross-attention layers.

In some embodiments, the at least one trained adapter comprises at least one linear projection layer and at least one non-linear activation layer.

In some embodiments, the trained multimodal generative model comprises a trained denoising model, and wherein generating the tissue image for the subject further comprises iteratively denoising a noise input using the trained denoising model conditioned on the adapted representation of the RNA expression data.

In some embodiments, the trained denoising model comprises a plurality of cross-attention layers, and wherein processing the adapted representation of the RNA expression data using the trained denoising model comprises processing the adapted representation of the RNA expression data using the plurality of cross-attention layers.

In some embodiments, the trained denoising model comprises a pre-trained backbone and a plurality of supplementary cross-attention layers, the pre-trained backbone comprising a plurality of original cross-attention layers.

In some embodiments, processing the adapted representation of the RNA expression data using the plurality of cross-attention layers comprises processing the adapted representation of the RNA expression data using the plurality of supplementary cross-attention layers and the plurality of original cross-attention layers.

In some embodiments, the pre-trained backbone comprises a UNet trained on a corpus of images.

Some embodiments further comprise training a denoising model to obtain the trained denoising model, wherein the training comprises updating weights of the supplementary cross-attention layers, and wherein the pre-trained backbone is at least partially frozen during training. Some embodiments further comprise training a denoising model to obtain the trained denoising model, wherein the training comprises sampling noise according to a denoising diffusion probabilistic model (DDPM) noise schedule and training the denoising model to predict the sampled noise.

In some embodiments, generating the tissue image comprises iteratively denoising a noise input using the trained denoising model according to a denoising diffusion implicit model (DDIM) sampling procedure.

In some embodiments, the trained denoising model uses between 50 and 100 diffusion steps.

In some embodiments, the trained multimodal generative model further comprises a decoder configured to process an output of the trained denoising model and to output the tissue image for the subject.

In some embodiments, the decoder is a decoder portion of a variational autoencoder (VAE) of a pre-trained latent diffusion model.

Some embodiments further comprise training the multimodal generative model jointly with the adapter.

Some embodiments further comprise training the multimodal generative model after training the encoder.

In some embodiments, the tissue image is a hematoxylin and eosin (H&E) image.

In some embodiments, the tissue image is at least 128×128 pixels and at most 2,048×2,048 pixels.

Some embodiments further comprise generating multiple tissue images for the subject from the RNA expression data.

In some embodiments, generating the multiple tissue images from the RNA expression data comprises processing, using the trained multimodal generative model conditioned on the adapted representation of the RNA expression data, a respective noise input for each of the multiple tissue images, the respective noise inputs being different from one another.

Some embodiments further comprise training the multimodal generative model to generate tissue images from RNA expression data on training data comprising paired RNA expression training data and tissue image training data.

In some embodiments, the RNA expression training data comprises an RNA expression training sample and the tissue image training data comprises a tissue image sample paired with the RNA expression training sample, wherein training the multimodal generative model to generate the tissue images comprises: encoding the RNA expression training sample using a trained encoder to obtain a representation of the RNA expression training sample; processing the representation of the RNA expression training sample using the adapter to obtain an adapted representation of the RNA expression training sample; and iteratively: selecting a timestep from a range of timesteps; applying noise to the tissue image training sample to obtain noised tissue image training sample; computing a predicted noise by processing, using the multimodal generative model, the noised tissue image training sample, the selected timestep, and the adapted representation of the RNA expression training sample; and updating weights of the multimodal generative model based on a difference between the noise applied to the tissue image training sample and the predicted noise.

Some embodiments further comprise identifying the subject as a member of a clinical trial cohort based on the tissue image and/or the RNA expression data.

Some embodiments further comprise identifying a treatment for the subject based on the tissue image and/or the RNA expression data.

Some embodiments further comprise administering the identified treatment to the subject. Some embodiments provide for a method for generating, from a tissue image, a representation of RNA expression data for a subject and an output based on the representation of RNA expression data, the method comprising: using at least one processor to perform: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some embodiments provide for a system comprising: at least one processor; and at least one non-transitory computer-readable storage medium storing processor-executable instructions that, when executed by the at least one processor, cause the at least one processor to perform a method for generating, from a tissue image, a representation of RNA expression data for a subject and an output based on the representation of RNA expression data, the method comprising: a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some embodiments provide for at least one non-transitory computer-readable storage medium storing processor-executable instructions that, when executed by at least one processor, cause the at least one processor to perform a method for generating, from a tissue image, a representation of RNA expression data for a subject and an output based on the representation of RNA expression data, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; generating the representation of the RNA expression data for the subject by processing, using the trained multimodal generative model, the adapted representation of the tissue image; and generating the output from the representation of the RNA expression data.

Some embodiments further comprise: excluding, from the image portions of the plurality of image portions, image portions that depict less than a threshold amount of tissue.

In some embodiments, an image portion of the plurality of image portions is at least 128×128 pixels and at most 2,048×2,048 pixels.

In some embodiments, the tissue image is a hematoxylin and eosin (H&E) image.

In some embodiments, the tissue image is a whole slide image (WSI), and wherein the plurality of image portions are tiles of the WSI.

In some embodiments, the trained encoder is an image encoder.

In some embodiments, the image encoder is a vision transformer (ViT).

In some embodiments, the ViT was trained using self-supervised learning.

In some embodiments, the ViT was trained using a self-distillation with no labels (DINO) method or a DINOv2 method.

In some embodiments, the ViT was pre-trained on a corpus of histopathology images.

In some embodiments, the image portions include a first image portion and a second image portion, and wherein encoding the image portions using the trained encoder to obtain the respective representations of the image portions comprises: encoding the first image portion using the trained encoder to obtain a first representation of the first image portion; and encoding the second image portion using the trained encoder to obtain a second representation of the second image portion.

In some embodiments, each of the representations of the image portions has a dimensionality of at least 256 and at most 2048, optionally wherein the dimensionality is 384.

In some embodiments, the representations of the image portions form a set of representations, and wherein the trained adapter is configured to transform the set of representations to the adapted representation of the tissue image.

In some embodiments, the trained adapter is configured to determine a weighted combination of the representations of the image portions to obtain the adapted representation of the tissue image.

In some embodiments, the trained adapter is a gated attention network.

In some embodiments, the gated attention network comprises: a first branch comprising a first linear layer followed by a first non-linear activation layer; a second branch comprising a second linear layer followed by a second non-linear activation layer; a gating operation configured to combine outputs of the first branch and outputs of the second branch by element-wise multiplication; an attention scoring layer configured to produce a respective attention score for each of the representations of the image portions from an output of the gating operation; a normalization configured to normalize the attention scores across the representations of the image portions; and a weighted sum operation configured to combine the representations of the image portions using the normalized attention scores to obtain the adapted representation of the tissue image.

In some embodiments, generating the representation of the RNA expression data for the subject further comprises iteratively denoising a noise input using the trained multimodal generative model conditioned on the adapted representation of the tissue image.

In some embodiments, the trained multimodal generative model comprises a trained denoising model configured to operate in a latent space, the latent space being a latent space of a trained autoencoder for RNA expression data.

In some embodiments, the trained autoencoder for RNA expression data is a trained latent image autoencoder (LIAE), and wherein the latent space comprises 1-channel, two-dimensional feature maps.

In some embodiments, the latent space comprises 1×32×32 feature maps.

In some embodiments, the trained multimodal generative model comprises a U-Net comprising a plurality of cross-attention layers, and wherein processing the adapted representation of the tissue image using the trained generative model comprises providing the adapted representation of the tissue image as a key-value input to the plurality of cross-attention layers.

In some embodiments, the U-Net comprises a plurality of down-sampling blocks, a middle block, and a plurality of up-sampling blocks, with cross-attention layers in at least one of the down-sampling blocks, the middle block, and at least one of the up-sampling blocks.

Some embodiments further comprise training the multimodal generative model to obtain the trained multimodal generative model, wherein the training comprises sampling noise according to a denoising diffusion probabilistic model (DDPM) noise schedule and training the multimodal generative model to predict the sampled noise.

In some embodiments, generating the representation of the RNA expression data comprises iteratively denoising a noise input using the trained multimodal generative model according to a denoising diffusion implicit model (DDIM) sampling procedure.

In some embodiments, the trained multimodal generative model uses between 100 and 300 diffusion steps.

Some embodiments further comprise training a multimodal generative model jointly with an adapter to obtain the trained multimodal generative model and the trained adapter.

In some embodiments, the trained encoder is frozen during the training of the multimodal generative model and the adapter.

Some embodiments further comprise training the multimodal generative model after training the encoder.

In some embodiments, the representation of the RNA expression data has a dimensionality of 1×32×32.

In some embodiments, generating the output from the representation of the RNA expression data comprises processing the representation of the RNA expression data using a trained decoder.

In some embodiments, the trained decoder comprises one or more convolutional layers.

In some embodiments, the trained decoder comprises a trained LIAE decoder.

In some embodiments, the trained LIAE decoder corresponds to a trained LIAE encoder, the trained LIAE encoder and the trained LIAE decoder having been jointly trained as an autoencoder on RNA expression data.

In some embodiments, the output comprises transcriptomic data for the subject.

In some embodiments, the transcriptomic data comprises a plurality of RNA expression levels for a respective plurality of genes.

Some embodiments further comprise determining, from the plurality of RNA expression levels, one or more signature scores for a respective one or more gene groups.

In some embodiments, the plurality of genes comprises at least 5,000 genes, at least 10,000 genes, at least 15,000 genes, or at least 20,000 genes.

In some embodiments, the output comprises one or more signature scores for a respective one or more gene groups.

In some embodiments, the output comprises a classification, prediction, or score for the subject derived from the representation of the RNA expression data.

Some embodiments further comprise generating multiple representations of the RNA expression data for the subject by processing, using the trained multimodal generative model conditioned on the adapted representation of the tissue image, a respective noise input for each of the multiple representations, the respective noise inputs being different from one another.

Some embodiments further comprise combining the multiple representations of the RNA expression data to obtain a combined representation of the RNA expression data.

Some embodiments further comprise identifying a treatment for the subject based on the tissue image and/or the representation of the RNA expression data.

Some embodiments further comprise administering the identified treatment to the subject.

Some embodiments further comprise identifying the subject as a member of a clinical trial cohort based on the tissue image and/or the representation of the RNA expression data.

In some embodiments, the trained multimodal generative model and the trained adapter are trained using paired tissue image data and RNA expression data, the RNA expression data being obtained by spatial transcriptomics.

Some embodiments provide for a method for generating, from a tissue image, a plurality of output values for a subject, the method comprising: using at least one processor to perform: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some embodiments provide a system comprising: at least one processor; and at least one non-transitory computer-readable storage medium storing processor-executable instructions that, when executed by the at least one processor, cause the at least one processor to perform a method for generating, from a tissue image, a plurality of output values for a subject, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some embodiments provide at least one non-transitory computer-readable storage medium storing processor-executable instructions that, when executed by at least one processor, cause the at least one processor to perform a method for generating, from a tissue image, a plurality of output values for a subject, the method comprising: obtaining a tissue image of a biological sample previously obtained from a subject, the tissue image comprising a plurality of image portions; encoding image portions of the plurality of image portions using a trained encoder to obtain respective representations of the image portions; processing the representations of the image portions using a trained adapter to obtain an adapted representation of the tissue image for subsequent processing using a trained multimodal generative model; and generating the plurality of output values for the subject by processing, using the trained multimodal generative model, the adapted representation of the tissue image.

Some embodiments further comprise: excluding, from the image portions of the plurality of image portions, image portions that depict less than a threshold amount of tissue.

In some embodiments, an image portion of the plurality of image portions is at least 128×128 pixels and at most 2,048×2,048 pixels.

In some embodiments, the tissue image is a hematoxylin and eosin (H&E) image.

In some embodiments, the tissue image is a whole slide image (WSI), and wherein the plurality of image portions are tiles of the WSI.

In some embodiments, the trained encoder is a vision transformer (ViT).

In some embodiments, a representation of an image portion has a dimensionality of between 256 and 2,000.

In some embodiments, the image portions include a first image portion and a second image portion, and wherein encoding the image portions using the encoder to obtain the respective representations of the image portions comprises: encoding the first image portion using the encoder to obtain a first representation of the first image portion; and encoding the second image portion using the encoder to obtain a second representation of the second image portion.

In some embodiments, the representations of the image portions form a set of representations, and wherein the trained adapter is configured to transform the set of representations to the adapted representation of the tissue image.

In some embodiments, the trained adapter is configured to determine a weighted combination of the representations of the image portions to obtain the adapted representation of the tissue image.

In some embodiments, the trained adapter comprises a feature extraction network and at least one gated attention network.

In some embodiments, a representation of an image portion has a first dimensionality, and wherein the feature extraction network is configured to map the representation of the image portion to an updated representation having a second dimensionality different from the first dimensionality. In some embodiments, the second dimensionality is between 128 and 2,048.

In some embodiments, the at least one gated attention network is configured to determine weights for the image portions.

In some embodiments, the trained adapter is configured to determine a combination of outputs of the feature extraction network using the weights determined by the at least one gated attention network.

In some embodiments, the trained adapter comprises a respective gated attention network for each output value of the plurality of output values.

In some embodiments, the at least one gated attention network comprises two parallel branches that each process an input representation, a gating operation that combines outputs of the two parallel branches, and an attention scoring layer configured to produce a respective attention weight for each of the image portions from an output of the gating operation.

In some embodiments, the trained multimodal generative model comprises a regression head trained to generate the plurality of output values.

In some embodiments, the trained multimodal generative model comprises multiple regression heads, wherein each of the multiple regression heads is trained to predict a respective one of the plurality of output values.

In some embodiments, the plurality of output values comprises signature scores for a respective plurality of gene groups.

In some embodiments, generating the signature scores for the plurality of gene groups comprises generating signature scores for at least two of the gene groups listed in Table 2.

Some embodiments further comprise generating a heatmap using the tissue image and an output of the trained adapter, wherein the heatmap indicates regions of the tissue image that contribute to an output value of the plurality of output values generated by the trained multimodal generative model.

In some embodiments, the output of the trained adapter comprises respective attention weights for at least some of the image portions, and wherein generating the heatmap comprises projecting the attention weights onto the tissue image.

Some embodiments further comprise generating a respective heatmap for each of at least some of the plurality of output values.

Some embodiments further comprise training the trained adapter and the trained multimodal generative model jointly on training data comprising a plurality of training tissue images and respective ground-truth values, wherein the trained encoder is frozen during training.

Some embodiments further comprise identifying a treatment for the subject based on the tissue image and/or the plurality of output values.

Some embodiments further comprise administering the identified treatment to the subject. Some embodiments further comprise identifying the subject as a member of a clinical trial cohort based on the tissue image and/or the plurality of output values.

Some embodiments provide for a method for training a multimodal generative model to generate data of a target modality for a subject, the method comprising: using at least one processor to perform: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

Some embodiments provide for a system comprising: at least one processor; and at least one when executed by at least one processor, cause the at least one processor to perform a method for training a multimodal generative model to generate data of a target modality for a subject, the method comprising: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

Some embodiments provide at least one non-transitory computer-readable storage medium storing processor-executable instructions that, when executed by at least one processor, cause the at least one processor to perform a method for training a multimodal generative model to generate data of a target modality for a subject, the method comprising: obtaining target training data of the target modality; obtaining representations of input training data of one or more modalities, the one or more modalities excluding the target modality; processing one or more of the representations of the input training data using at least one adapter to obtain one or more adapted representations of the input training data; and iteratively: selecting a timestep from a range of timesteps; applying noise to the target training data to obtain noised target training data; computing a predicted noise by processing, using the multimodal generative model, the noised target training data, the selected timestep, and the one or more adapted representations of the input training data; and updating weights of the multimodal generative model based on a difference between the noise applied to the target training data and the predicted noise.

In some embodiments, the iteratively further comprises updating weights of the at least one adapter, based on the difference between the noise applied to the target training data and the predicted noise, to obtain at least one trained adapter.

In some embodiments, the at least one adapter comprises a respective adapter for each of the one or more modalities.

In some embodiments, obtaining the one or more representations of the input training data comprises encoding the input training data of the one or more modalities using at least one trained encoder to obtain the one or more representations of the input training data.

In some embodiments, the at least one trained encoder has been trained prior to training the multimodal generative model.

In some embodiments, the at least one trained encoder has been trained using training data that comprises data not included in the target training data or the input training data.

In some embodiments, the at least one trained encoder comprises a respective trained encoder for each of the one or more modalities.

In some embodiments, each of the one or more modalities has a respective trained encoder and a respective adapter.

Some embodiments further comprise, during the iteratively updating, masking and/or omitting at least one modality of the one or more modalities provided to the multimodal generative model for at least some of the training iterations, the multimodal generative model thereby being trained to generated the target modality with and without each of the one or more modalities.

Some embodiments further comprise encoding the target training data using a second trained encoder to obtain encoded target training data, wherein applying noise to the target training data comprises applying noise to the encoded target training data, and wherein the multimodal generative model operates in a latent space of the second trained encoder.

Some embodiments further comprise training the second encoder prior to training the multimodal generative model.

Some embodiments further comprise training a second trained decoder paired with the second trained encoder, the second trained encoder and the second trained decoder together comprising an autoencoder trained on data of the target modality.

In some embodiments, applying the noise to the target training data comprises sampling noise according to a denoising diffusion probabilistic model (DDPM) forward noise process.

In some embodiments, the target training data and the input training data is paired training data.

In some embodiments, the multimodal generative model comprises a pre-trained backbone and one or more additional layers, and wherein updating the weights of the multimodal generative model comprises updating weights of the one or more additional layers while keeping weights of the pre-trained backbone frozen.

Some embodiments provide for a trained multimodal generative model obtained by the methods described herein.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within +20% of a target value in some embodiments, within +10% of a target value in some embodiments, within +5% of a target value in some embodiments, within +2% of a target value in some embodiments. The terms “approximately,” “substantially,” and “about” may include the target value.

What is claimed is:

1. A method for generating, from RNA expression data, a tissue image for a subject, the method comprising:
using at least one processor to perform:
obtaining RNA expression data derived from a biological sample previously obtained from a subject;
encoding the RNA expression data using a trained encoder to obtain a representation of the RNA expression data;
processing the representation of the RNA expression data using a trained adapter to obtain an adapted representation of the RNA expression data for subsequent processing using a trained multimodal generative model; and
generating the tissue image for the subject by processing, using the trained multimodal generative model, the adapted representation of the RNA expression data.

2. The method of claim 1, wherein obtaining the RNA expression data comprises obtaining a plurality of expression level values for a respective plurality of genes, wherein the plurality of genes comprises at least 5,000 genes, at least 10,000 genes, at least 15,000 genes, at least 20,000 genes, or any number of genes between 5,000 and 25,000.

3. The method of claim 2, further comprising modifying one or more of the expression level values prior to encoding the RNA expression data.

4. The method of claim 1, wherein the trained encoder is an autoencoder.

5. The method of claim 4, wherein the autoencoder is an encoder portion of a latent image autoencoder (LIAE) or an encoder portion of a vector quantized variational autoencoder (VQ-VAE).

6. The method of claim 5, wherein the autoencoder is the encoder portion of the LIAE, and wherein the encoder portion of the LIAE comprises:
a projection block configured to project the RNA expression data to an intermediate representation;
a reshaping operation configured to reshape the intermediate representation into a two-dimensional feature map; and
a plurality of residual blocks configured to process the two-dimensional feature map.

7. The method of claim 1, wherein the trained adapter is configured to map the representation of the RNA expression data to the adapted representation of the RNA expression data having a dimensionality matching a conditioner input of the trained multimodal generative model.

8. The method of claim 1, wherein the trained adapter comprises at least one linear projection layer and at least one non-linear activation layer.

9. The method of claim 1, wherein the trained multimodal generative model comprises a trained denoising model, and wherein generating the tissue image for the subject further comprises iteratively denoising a noise input using the trained denoising model conditioned on the adapted representation of the RNA expression data.

10. The method of claim 9, wherein the trained denoising model comprises a plurality of cross-attention layers, and wherein processing the adapted representation of the RNA expression data using the trained denoising model comprises processing the adapted representation of the RNA expression data using the plurality of cross-attention layers.

11. The method of claim 10, wherein the trained denoising model comprises a pre-trained backbone and a plurality of supplementary cross-attention layers, the pre-trained backbone comprising a plurality of original cross-attention layers.

12. The method of claim 9, wherein the trained multimodal generative model further comprises a decoder configured to process an output of the trained denoising model and to output the tissue image for the subject.

13. The method of claim 1, wherein the tissue image is a hematoxylin and eosin (H&E) image.

14. The method of claim 1, further comprising generating multiple tissue images for the subject from the RNA expression data, the generating comprising: processing, using the trained multimodal generative model conditioned on the adapted representation of the RNA expression data, a respective noise input for each of the multiple tissue images, the respective noise inputs being different from one another.

15. The method of claim 1, further comprising training the multimodal generative model to generate tissue images from RNA expression data on training data comprising paired RNA expression training data and tissue image training data.

16. The method of claim 1, further comprising identifying the subject as a member of a clinical trial cohort based on the tissue image and/or the RNA expression data.

17. The method of claim 1, further comprising identifying a treatment for the subject based on the tissue image and/or the RNA expression data.

18. The method of claim 17, further comprising administering the identified treatment to the subject.

19. A system comprising:
at least one processor; and
at least one non-transitory computer-readable storage medium storing processor-executable instructions that, when executed by the at least one processor, cause the at least one processor to perform method for generating, from RNA expression data, a tissue image for a subject, the method comprising:
obtaining RNA expression data derived from a biological sample previously obtained from a subject;
encoding the RNA expression data using a trained encoder to obtain a representation of the RNA expression data;
processing the representation of the RNA expression data using a trained adapter to obtain an adapted representation of the RNA expression data for subsequent processing using a trained multimodal generative model; and
generating the tissue image for the subject by processing, using the trained multimodal generative model, the adapted representation of the RNA expression data.

20. At least one non-transitory computer-readable storage medium storing processor-executable instructions that, when executed by at least one processor, cause the at least one processor to perform a method for generating, from RNA expression data, a tissue image for a subject, the method comprising:
obtaining RNA expression data derived from a biological sample previously obtained from a subject;
encoding the RNA expression data using a trained encoder to obtain a representation of the RNA expression data;
processing the representation of the RNA expression data using a trained adapter to obtain an adapted representation of the RNA expression data for subsequent processing using a trained multimodal generative model; and
generating the tissue image for the subject by processing, using the trained multimodal generative model, the adapted representation of the RNA expression data.
